SEQUENCE LISTING (51 sequences)

SEQ ID NO: 1 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSVAGPSNMAVQGRNWLPGPCYRQQRVSKTSADNNNSEYAWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctgtggccggaccgagtaacatggcggtccagggtaggaactggcttcctggaccctgttaccgccagcagcgagtatcaaagacatctgcggataacaacaacagtgaatacgcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - DNA, 6118 bp, synthetic construct
  source 1..6118: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagcttcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggatatcggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcgggactgccgcagggggacggctgccttcgggggggacggggcagggcggggttcggcttctggcgtgtgaccggcggctctagagcctctgctaaccatgttcatgccttcttctttttcctacagaccggtctcctgggcaacgtgctggttattgtgctgtctcatcattttggcaaagaattccaccgccaccatgagccggaagatcgagggcttcctgctgctgctgctgtttggatacgaggccaccctgggactgtcctccaccgaggacgagggcgaggacccctggtaccagaaggcctgcaagtgcgactgccagggcgggcctaacgccctgtggagcgctggagctaccagcctggactgcattcccgagtgcccatatcacaagcccctgggattcgagagcggagaggtgaccccagaccagattacatgcagtaacccagaacagtatgtgggatggtattcaagctggaccgccaacaaggccagactgaacagccagggcttcggctgcgcctggctgagcaagttccaggacagcagccagtggctgcagattgacctgaaggagatcaaggtgatctcagggatcctgacccagggaagatgcgacatcgatgagtggatgaccaagtacagcgtgcagtacagaaccgacgaaagactgaactggatctactacaaagaccagacaggaaacaacagagtgttctacggcaacagcgacagaaccagcaccgtgcagaacctgctgagaccccccatcatcagcagattcatcagactgatccccctggggtggcacgtgagaatcgcaatcagaatggagctgctggaatgcgtgagcaagtgcgcctgaggatccacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 4 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 6 - DNA, 276 bp, synthetic construct
  source 1..276: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 7 - DNA, 1009 bp, synthetic construct
  source 1..1009: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcgggactgccgcagggggacggctgccttcgggggggacggggcagggcggggttcggcttctggcgtgtgaccggcggctctagagcctctgctaaccatgttcatgccttcttctttttcctacag

SEQ ID NO: 8 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 9 - DNA, 816 bp, synthetic construct
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcat

SEQ ID NO: 10 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 144 bp, synthetic construct
  source 1..144: mol_type = other DNA
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 12 - DNA, 134 bp, synthetic construct
  source 1..134: mol_type = other DNA
atgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 13 - DNA, 113 bp, synthetic construct
  source 1..113: mol_type = other DNA
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 14 - DNA, 5488 bp, synthetic construct
  source 1..5488: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggatatcggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagaccggtctcctgggcaacgtgctggttattgtgctgtctcatcattttggcaaagaattccaccgccaccatgagccggaagatcgagggcttcctgctgctgctgctgtttggatacgaggccaccctgggactgtcctccaccgaggacgagggcgaggacccctggtaccagaaggcctgcaagtgcgactgccagggcgggcctaacgccctgtggagcgctggagctaccagcctggactgcattcccgagtgcccatatcacaagcccctgggattcgagagcggagaggtgaccccagaccagattacatgcagtaacccagaacagtatgtgggatggtattcaagctggaccgccaacaaggccagactgaacagccagggcttcggctgcgcctggctgagcaagttccaggacagcagccagtggctgcagattgacctgaaggagatcaaggtgatctcagggatcctgacccagggaagatgcgacatcgatgagtggatgaccaagtacagcgtgcagtacagaaccgacgaaagactgaactggatctactacaaagaccagacaggaaacaacagagtgttctacggcaacagcgacagaaccagcaccgtgcagaacctgctgagaccccccatcatcagcagattcatcagactgatccccctggggtggcacgtgagaatcgcaatcagaatggagctgctggaatgcgtgagcaagtgcgcctaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 15 - protein, 224 aa, synthetic construct
MSRKIEGFLLLLLFGYEATLGLSSTEDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 16 - DNA, 675 bp, synthetic construct
  source 1..675: mol_type = other DNA
atgagccggaagatcgagggcttcctgctgctgctgctgtttggatacgaggccaccctgggactgtcctccaccgaggacgagggcgaggacccctggtaccagaaggcctgcaagtgcgactgccagggcgggcctaacgccctgtggagcgctggagctaccagcctggactgcattcccgagtgcccatatcacaagcccctgggattcgagagcggagaggtgaccccagaccagattacatgcagtaacccagaacagtatgtgggatggtattcaagctggaccgccaacaaggccagactgaacagccagggcttcggctgcgcctggctgagcaagttccaggacagcagccagtggctgcagattgacctgaaggagatcaaggtgatctcagggatcctgacccagggaagatgcgacatcgatgagtggatgaccaagtacagcgtgcagtacagaaccgacgaaagactgaactggatctactacaaagaccagacaggaaacaacagagtgttctacggcaacagcgacagaaccagcaccgtgcagaacctgctgagaccccccatcatcagcagattcatcagactgatccccctggggtggcacgtgagaatcgcaatcagaatggagctgctggaatgcgtgagcaagtgcgcctga

SEQ ID NO: 17 - DNA, 675 bp, synthetic construct
  source 1..675: mol_type = other DNA
atgtccaggaaaatcgaaggattcctgctgctgctgctgttcgggtacgaggccaccctgggactgtcctccaccgaggatgagggcgaagacccatggtatcagaaggcctgcaaatgcgactgtcagggcggccccaacgccctgtggtcagccggcgctaccagcctggactgcatcccagagtgcccctaccacaagcccctgggattcgaatccggcgaggtgaccccagaccagattacctgttccaatcccgagcagtatgtggggtggtattccagctggaccgccaacaaggccaggctgaactcacaggggttcggctgcgcctggctgagcaagttccaggattcctcccagtggctgcagattgacctgaaggagatcaaggtgatctccggcatcctgacccaggggaggtgtgacatcgacgaatggatgaccaagtacagcgtgcagtacagaaccgacgagaggctgaactggatctattataaggatcagacaggaaacaaccgcgtgttctacggtaactccgatagaacaagcacagtgcagaacctgctgcggcctccaatcatcagtcgatttattcggctgatccccctgggctggcacgtgcgcatcgccattaggatggagctgctggagtgtgtgagcaagtgcgcctga

SEQ ID NO: 18 - DNA, 678 bp, synthetic construct
  source 1..678: mol_type = other DNA
atgtacagaatgcagctgctgagctgcattgccctgagcctggccctggtgacaaactccctgggcctgtccagcacagaggatgagggagaggacccttggtatcagaaggcctgcaaatgcgactgccagggcgggccaaatgctctgtggagtgccggcgccacctccctggactgtatccccgagtgtccctaccacaagcccctgggcttcgagagcggcgaggtcacccctgatcagataacttgctccaaccccgagcagtatgtgggctggtatagcagctggaccgccaacaaagcccggctgaacagccagggcttcggctgcgcatggctgagcaagttccaggacagcagccagtggctgcagatcgacctgaaagagatcaaggtgatctccggcatcctgacccagggccggtgcgacattgacgagtggatgaccaagtactccgtgcagtatcgcaccgatgagcgcctgaattggatctattataaagaccagaccggaaacaacagagtgttttacggaaacagcgaccggaccagcactgtgcagaacctgctgcgccctccgatcatttctcggtttatcaggctgattcccctgggctggcacgtccggatcgccatccgcatggaactgctggagtgtgtgagtaagtgtgcctga

SEQ ID NO: 19 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - DNA, 675 bp, synthetic construct
  source 1..675: mol_type = other DNA
atgtcacgcaagatagaaggctttttgttattacttctctttggctatgaagccacattgggattatcgtctaccgaggatgaaggcgaggacccctggtaccaaaaagcatgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcagggatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 21 - DNA, 6148 bp, synthetic construct
  source 1..6148: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagcttcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggatatcggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggcccagccggcctcgcgagaattctctagaacgccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagagctgacactagtgcggatccacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 22 - DNA, 493 bp, synthetic construct
  source 1..493: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 23 - DNA, 924 bp, synthetic construct
  source 1..924: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatccgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctaagcttctgcaggtcgactctagaggatccaccggtctcctgggcaacgtgctggttattgtgctgtctcatcattttggcaaagaattc

SEQ ID NO: 24 - DNA, 431 bp, synthetic construct
  source 1..431: mol_type = other DNA
caagctgtgaccggcgcctacgatatccgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctaagcttctgcaggtcgactctagaggatccaccggtctcctgggcaacgtgctggttattgtgctgtctcatcattttggcaaagaattc

SEQ ID NO: 25 - DNA, 640 bp, synthetic construct
  source 1..640: mol_type = other DNA
ggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatccgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctaagcttctgcaggtcgactctagaggatcc

SEQ ID NO: 26 - DNA, 400 bp, synthetic construct
  source 1..400: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 27 - DNA, 1648 bp, synthetic construct
  source 1..1648: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaac

SEQ ID NO: 28 - DNA, 1248 bp, synthetic construct
  source 1..1248: mol_type = other DNA
gagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaac

SEQ ID NO: 29 - DNA, 660 bp, synthetic construct
  source 1..660: mol_type = other DNA
acgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 30 - DNA, 1160 bp, synthetic construct
  source 1..1160: mol_type = other DNA
acgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 31 - DNA, 500 bp, synthetic construct
  source 1..500: mol_type = other DNA
gcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 32 - DNA, 582 bp, synthetic construct
  source 1..582: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 33 - DNA, 42 bp, synthetic construct
  source 1..42: mol_type = other DNA
actaggggttcctgcggccgcacgcgtggaattcgctagtta

SEQ ID NO: 34 - DNA, 34 bp, synthetic construct
  source 1..34: mol_type = other DNA
gccacccgtagatctacggtatcatatcgataag

SEQ ID NO: 35 - DNA, 41 bp, synthetic construct
  source 1..41: mol_type = other DNA
actaggggttcctgcggccgcggcctccgcgccgggttttg

SEQ ID NO: 36 - DNA, 41 bp, synthetic construct
  source 1..41: mol_type = other DNA
gccacccgtagatctctcgagttaggcgcacttgctcacgc

SEQ ID NO: 37 - DNA, 41 bp, synthetic construct
  source 1..41: mol_type = other DNA
actaggggttcctgcggccgcacgcgtctagttattaatag

SEQ ID NO: 38 - DNA, 40 bp, synthetic construct
  source 1..40: mol_type = other DNA
ccacccgtagatctctcgagcagcgctgctcgaggcaagc

SEQ ID NO: 39 - DNA, 36 bp, synthetic construct
  source 1..36: mol_type = other DNA
tcactaggggttcctatcgatatcaagcttcgttac

SEQ ID NO: 40 - DNA, 34 bp, synthetic construct
  source 1..34: mol_type = other DNA
agggatgccacccgtggatcctcaggcgcacttg

SEQ ID NO: 41 - DNA, 44 bp, synthetic construct
  source 1..44: mol_type = other DNA
ttcgaacatcgattgaattctgagccaccatgtccaggaaaatc

SEQ ID NO: 42 - DNA, 40 bp, synthetic construct
  source 1..40: mol_type = other DNA
aggtcgactctagaggatcctcaggcgcacttgctcacac

SEQ ID NO: 43 - DNA, 41 bp, synthetic construct
  source 1..41: mol_type = other DNA
ttcgaacatcgattgaattctgagccaccatgtacagaatg

SEQ ID NO: 44 - DNA, 40 bp, synthetic construct
  source 1..40: mol_type = other DNA
aggtcgactctagaggatcctcaggcacacttactcacac

SEQ ID NO: 45 - DNA, 42 bp, synthetic construct
  source 1..42: mol_type = other DNA
gactctagaggatcgaattcgccaccatgtcacgcaagatag

SEQ ID NO: 46 - DNA, 39 bp, synthetic construct
  source 1..39: mol_type = other DNA
gggatgccacccgtggatcctcaggcacacttgctgacg

SEQ ID NO: 47 - DNA, 39 bp, synthetic construct
  source 1..39: mol_type = other DNA
caagttaacaacaacaattgcattcattttatgtttcag

SEQ ID NO: 48 - DNA, 39 bp, synthetic construct
  source 1..39: mol_type = other DNA
acggccagtgaattgtcgacgcatgcgggcccggatctg

SEQ ID NO: 49 - DNA, 25 bp, synthetic construct
  source 1..25: mol_type = other DNA
gacgtcagacgcggaagcttcgatc

SEQ ID NO: 50 - DNA, 24 bp, synthetic construct
  source 1..24: mol_type = other DNA
gctgtttaaacgcccgggctgtag

SEQ ID NO: 51 - protein, 735 aa, synthetic construct [sequence omitted]

(19) United States
(12) Patent Application Publication
(10) Pub. No.: US 2026/0226111 A1
(43) Pub. Date: Aug. 6, 2026

(54) NOVEL CAPSID PROTEIN VARIANT AND USE THEREOF

(71) Applicant: SHANGHAI LANGSHENG BIOTECHNOLOGY CO., LTD, Shanghai (CN)

(72) Inventors: Fei WU, Shanghai (CN); Zhuoyu NI, Shanghai (CN); Fenghua WANG, Shanghai (CN)

(21) Appl. No.: 19/149,466

(22) PCT Filed: Jan. 16, 2024
(86) PCT No.: PCT/CN2024/072557

§ 371 Date: Jul. 18, 2025

(30) Foreign Application Priority Data
Jan. 18, 2023 (CN) 202310086693.X

(51) Int. Cl. C07K 14/005; A61K 38/17; A61K 48/00; A61P 27/02; C07K 14/705; C12N 15/86

(52) U.S. Cl. CPC C07K 14/005; A61K 38/178; A61K 48/0033; A61K 48/005; A61K 48/0075; A61K 48/0083; A61P 27/02; C07K 14/7056; C12N 15/86; C12N 2750/14122; C12N 2750/14143; C12N 2750/14152

(57) ABSTRACT

An capsid protein variants, recombinant AAV viral particles comprising the same, and a method of using the recombinant AAV viral particles for treating an ocular disease, said capsid protein variants comprises the following substitutions as compared to the parental AAV capsid protein VP1: Q464V, A467P, D469N, 1470M, R471A, D472V, S474G, optionally Y500F and/or S501A; said ocular disease is X-linked hereditary retinoschisis.

The present invention relates to a capsid protein variant, recombinant AAV viral particles comprising the same, and method of using the recombinant AAV viral particles for treating an ocular disease.

### BACKGROUND OF THE INVENTION

Currently, there are about 15 million people with blindness caused by inherited retinal diseases in the world, accounting for about 0.02% of the total population. There are many types of inherited retinal diseases (IRDs), mainly including non-syndromic retinitis pigmentosa (40%), Usher syndrome (10%), cone-rod dystrophy (10%), Leber congenital amaurosis (5%), Leber hereditary optic neuropathy and major subtypes.

The leading cause of macular degeneration in male adolescents is X-linked hereditary retinoschisis. As early as 1898, ophthalmologist Hass from German described two cases of typical radial cystoid macular degeneration in men, which were later confirmed as X-linked genetic retinoschisis. The clinical manifestations of XLRS are highly variable, but are mainly characterized by microcystic wheel-like changes in the macular area due to inner retinal rupture, with peripheral bullous lesions in about 50% of cases. Most patients develop progressive visual impairment during school age. In the later stages of the disease, the vision may be significantly reduced or even blind due to complications such as vitreous hemorrhage, choroidal sclerosis, retinal detachment, and retinal atrophy. Electroretinogram (ERG) examination shows that the b wave originating from the inner retina decreases while the a wave representing the photoreceptors remains relatively normal or slightly decreases, resulting in a decrease in the b/a ratio, which is a characteristic electrophysiological change of this disease. In addition, interference optical tomography (OCT) shows nerve fiber layer fissures, and fundus photography and fluorescein angiography (FFA) are also helpful in the diagnosis of this disease. This disease occurs all over the world, with a prevalence of 1:5000 to 1:25000. It is inherited in an X-linked recessive manner. Males are affected, and females can be carriers without clinical manifestations. The RS1 gene was identified by Professor Bernard Weber through positional cloning in 1997 (Sauer et al., Nat Genet). The human RS1 protein that contains only 224 amino acids is an extracellular matrix molecule with a secretion signal peptide. The C-terminal discoid domain of the RS1 protein performs the “adhesion function” among cells.

With the successful construction of human genetic maps and the rapid development of molecular biology techniques, the viral vectors have been applied successfully, which led to certain progress in the gene therapy of XLRS. Many experimental studies have laid a lot of groundwork and preliminary work for the clinical application of gene therapy for XLRS.

However, at present, there is no specific drug or treatment for XLRS juvenile retinoschisis in clinical practice. Most of the existing technologies for XLRS gene therapy use CMV promoters. In primates and clinical trials, CMV promoters are at risk of being silenced and are toxic to the photoreceptors and retinal pigment epithelial cells of the eyes. At the same time, the serotypes disclosed in the prior art of XLRS gene therapy products are generally AAV2, AAV8 and AAV2.7m8. When administered through the vitreous cavity, the serotypes disclosed in the prior art cannot efficiently penetrate the inner limiting membrane of the retina, resulting in most rAAV remaining in the vitreous humor, inducing intraocular inflammation and affecting the clinical treatment effect of XLRS. The serotype disclosed in the present invention can efficiently penetrate the inner limiting membrane and finally reach the photoreceptor cells, and can secrete and express sufficient RS1 multimeric protein to play the role of a scaffold to treat retinoschisis. For XLRS gene therapy, not only the effectiveness of the treatment, but also the long-term effect of the therapeutic drugs should be considered. Since most patients often develop the disease since childhood, while considering the effectiveness, achieving the long-term effect of drug treatment and solving the convenience of drug administration in clinical trials are the keys to improve patient compliance and are also issues that urgently need to be addressed.

AAV is a small, non-enveloped, single-stranded DNA virus. The AAV genome is 4.7 kb and is characterized by two Inverted Terminal Repeats (ITRs), and two open reading frames encoding Rep and Cap proteins, respectively. These two ITRs are the only cis-elements required for AAV replication, packaging, and integration. The Rep reading frame encodes four proteins with molecular weights of 78 kD, 68 kD, 52 kD, and 40 kD. These proteins primarily play a role in regulating AAV replication and integration of AAV into the chromosomes of host cells. The Cap reading frame encodes three structural (capsid) Viral Proteins (VP) with molecular weights of 83-85 kD (VP1), 72-73 kD (VP2), and 61-62 kD (VP3). More than 80% of the total protein in AAV virions comprises VP3; in mature virions, VP1, VP2, and VP3 were found at relative abundances of approximately 1:1:10. In vitro, these three proteins spontaneously assemble into virosome-like structures, such as viral capsids. Thus, it appears that viral capsid formation in infected cells proceeds independently of viral DNA synthesis.

AAV is a promising vector for human gene therapy because, unlike other viral vectors, AAV has not been shown to be associated with any known human disease, and is not generally considered pathogenic. Furthermore, AAV safely transduces postmitotic tissue with relatively low immunogenicity, and is able to integrate into host chromosomes in a site-specific manner and into chromosome 19 in tissue culture cells if the Rep protein is supplied in trans. The integrated genome of AAV has been shown to allow long-term gene expression in many tissues, including muscle, liver, and brain.

There is an urgent need in the art to develop a novel AAV, as well as a safe and reliable therapeutic method or drug based thereon for the effective treatment of XLRS, e.g., a therapeutic vector or composition for gene therapy.

### SUMMARY OF THE INVENTION

The present invention relates to a gene therapy drug (such as an AAV virus particle) which is based on a directed evolution mutant of recombinant adeno-associated virus serotype 2 and is used as a vector carrying a human RS1 gene (hRS1).

In the present invention, the AAV virus particles, due to their unique capsid and optimized RS1 gene, exhibited efficient transduction activity in photoreceptor cells in vitro. At the same time, compared with the reference product, the AAV virus particles show significantly improved transduction activity in various layers of retinal tissue in mice under different in vivo administration modes. At the same time, compared with the existing serotype AAV8 for the efficacy, under the same intravitreal administration (IVT) mode, the virus particles of the present invention significantly improved in terms of indicators such as in vivo transduction activity and gene therapy drug effectiveness.

Accordingly, the present invention relates to an AAV capsid protein variant comprising or consisting of a mutation, relative to a parental AAV capsid protein variant, selected from the group consisting of Q464V, A467P, D469N, 1470M, R471A, D472V, and S474G.

The present invention further relates to a recombinant AAV viral particle comprising
(i) an AAV capsid comprising an engineered capsid protein VP1 comprising, or comprising only, the following amino acid mutation sites relative to a parental AAV capsid protein VP1: Q464V, A467P, D469N, 1470M, R471A, D472V, and S474G;
(ii) an interested nucleic acid, e.g., a nucleic acid encoding RS1, packaged within the AAV capsid.

The present invention also relates to a pharmaceutical formulation, composition or medicament comprising a recombinant AAV viral particle of the present invention.

The present invention also relates to restoring vision, and/or treating or preventing an ocular disease in a subject, comprising administering to the subject a recombinant AAV viral particle of the present invention, or a pharmaceutical formulation, composition or medicament comprising the same.

In one aspect, the present invention relates to the following embodiments:
1. An AAV capsid protein variant comprising an motified capsid protein VP1 comprising the following amino acid mutation sites relative to a parental AAV capsid protein VP1: Q464V, A467P, D469N, 1470M, R471A, D472V, and S474G, wherein the positions are determined with reference to the amino acid sequence position of SEQ ID NO: 51.
2. The AAV capsid protein variant of embodiment 1, wherein the parental AAV capsid protein VP1 is from AAV serotype 2 (AAV2) or AAV2 modified version 7m8 (AAV2.7m8), preferably AAV2.7m8.
3. The AAV capsid protein variant of embodiment 1, wherein the AAV capsid protein variant
(i) comprises the amino acid sequence as set forth in SEQ ID NO:1.
(ii) comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:1 and comprises the following substitutions: Q464V, A467P, D469N, 1470M, R471A, D472V, and S474G;
(iii) is encoded by the nucleic acid sequence as set forth in SEQ ID NO: 2; or
(iv) is encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence set forth in SEQ ID NO:2 and comprises the following substitutions: Q464V, A467P, D469N, I470M, R471A, D472V, and S474G.
4. An isolated nucleic acid comprising a nucleotide sequence encoding the AAV capsid protein variant according to any one of embodiments 1-3.
5. The isolated nucleic acid according to embodiment 4, wherein the nucleotide sequence encodes a capsid protein, which
(i) comprises or consists of the nucleic acid sequence as set forth in SEQ ID NO:2; or
(iv) comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:2 and the capsid protein variant encoded by the same comprises the following substitutions: Q464V, A467P, D469N, 1470M, R471A, D472V, and S474G.
6. A recombinant AAV viral particle (rAAV) comprising
(i) an AAV capsid protein variant of any one of embodiments 1-5;
(ii) an interested nucleic acid packaged within the AAV capsid, the interested nucleic acid comprising a nucleic acid molecule encoding RS1.
7. The recombinant AAV viral particle of embodiment 6, wherein the RS1 is human RS1.
8. The recombinant AAV viral particle of embodiment 7, wherein the human RS1 protein
(i) comprises or consists of the amino acid sequence as set forth in SEQ ID NO: 15; or
(ii) comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:15.
9. The recombinant AAV viral particle of any one of embodiments 6-8, wherein the RS1 is encoded by the following coding sequence which
(i) comprises or consists of the nucleotide sequence as set forth in any one of SEQ ID NOs: 16-18 or a complementary sequence thereof; or
(ii) comprises a nucleotide sequence having at least 85%, 86%, 87%, 88%, 89%, 90%, 91%, 92%, 93%, 94%, 95%, 96%, 97%, 98%, or 99% identity to the nucleotide sequence as set forth in any one of SEQ ID NOs: 16-18 or a complementary sequence thereof.
10. The recombinant AAV viral particle according to any of embodiments 6-9, wherein the interested nucleic acid is comprised in an expression cassette and then packaged within the AAV capsid.
11. The recombinant AAV viral particle according to embodiment 10, wherein the expression cassette is single-stranded DNA, double-stranded DNA or single-stranded RNA or double-stranded RNA.
12. The recombinant AAV viral particle of embodiment 10 or 11, wherein the expression cassette comprises at least one ITR (e.g., L-ITR or R-ITR) sequence flanking the interested nucleic acid.
13. The recombinant AAV viral particle of any one of embodiments 10-12, wherein the expression cassette comprises one or more regulatory sequences selected from one or more or all of a promoter, an inverted repeat sequence, an intron, an enhancer, a post-transcriptional regulatory sequence, a polyadenylation region, a selectable marker, or a reporter gene.
14. A method of producing a recombinant AAV viral particle, comprising culturing a packaging cell under conditions sufficient for production of a recombinant AAV viral particle, wherein the packaging cell comprises a plasmid comprising a nucleic acid encoding the capsid protein variant of any one of embodiments 1-3 or the nucleic acid of embodiment 4 or 5.
15. The method according to embodiment 14, wherein the packaging cell further comprises a helper plasmid and/or a transfer plasmid comprising the interested nucleic acid.
16. The method according to embodiment 14 or 15, further comprising isolating from the culture supernatant a self-complementary recombinant AAV viral particle.
17. The method according to any one of embodiments 14-16, further comprising lysing the packaging cell, and isolating a recombinant AAV viral particle from the cell lysate.
18. The method according to any one of embodiments 14-16, further comprising one or more of the following steps:
a. removing the cell debris,
b. treating the supernatant containing the recombinant AAV viral particle with a benzonase nuclease,
c. concentrating the recombinant AAV viral particle,
d. purifying the recombinant AAV viral particle.
19. A recombinant AAV viral particle prepared according to the method of any one of embodiments 14-18.
20. A packaging cell for producing a recombinant AAV viral particle, the packaging cell comprising a plasmid comprising a nucleic acid encoding the capsid protein variant according to any one of embodiments 1-3 or the nucleic acid of embodiment 4 or 5.
21. A formulation or composition or medicament comprising the recombinant AAV viral particle of any one of embodiments 6-13 or 19, and optionally a pharmaceutically acceptable auxiliary material, such as a pharmaceutically acceptable carrier, an excipient, including a buffer, as known in the art.
22. A combination product comprising the recombinant AAV viral particle of any one of embodiments 6-13 or 19, and one or more additional therapeutic agents, e.g., an immunomodulator, such as, an immunosuppressant.
23. A method of treating an ocular disease in an individual comprising administering to the individual the recombinant AAV viral particle of any one of embodiments 6-13 or 19 or the formulation or composition or medicament of embodiment 21 or the combination product of embodiment 22.
24. The method of embodiment 23, wherein the ocular disease is selected from the group consisting of diseases caused by retinopathy or chorioretinopathy, age-related macular degeneration, diabetic retinopathy, and other non-hereditary ophthalmic diseases such as physically or chemically induced retinal damage, e.g., X-linked retinoschisis or retinitis pigmentosa.
25. The method of embodiment 23 or 24, wherein the administering is via intraocular administration, e.g., by intraretinal administration or intravitreal administration, e.g., subretinal administration or intravitreal administration (IVT).
26. The method of embodiment 25, wherein the administering is injection.
27. The method of any one of embodiments 23-26, wherein the recombinant AAV viral particle is administered at a dose of greater than or equal to about 10³vg/eye/time, 10⁴vg/eye/time, 10⁵vg/eye/time, 10⁶vg/eye/time, 10⁷vg/eye/time, 10⁸vg/eye/time, 10⁹vg/eye/time, 10¹⁰ vg/eye/time, 10¹¹vg/eye/time, or 10¹²vg/eye/time, preferably no more than 10¹²vg/eye/time, for example, greater than or equal to 2×10⁸vg/eye/time, 3×10⁸vg/eye/time, 4×10⁸vg/eye/time or 5×10⁸vg/eye/time, for example, between about 10⁷vg/eye/time and about 5×10⁸vg/eye/time, between 1×10⁷vg/eye/time and about 5×10⁸vg/eye/time, for example, about 2×10⁸vg/eye/time, 3×10⁸vg/eye/time, 4×10⁸vg/eye/time or 5×10⁸vg/eye/time.

### BRIEF DESCRIPTION OF THE DRAWINGS

FIG. 1: Comparison of expression efficiency of different expression cassettes, wherein FIG. 1A shows the structures for respective GOI plasmids; FIG. 1B shows the expression of hRS1 in the corresponding cell lysates and cell culture supernatants of 293T cells; and FIGS. 1C and 1D show the expression of RS1 of each plasmid in cells (C) and supernatants (D); FIG. 1E shows the expression of hRS1 in cell lysates and supernatants upon HEK293T cell expression; and FIGS. 1F and 1G show the relative expression levels of RS1 in lysates (F) and supernatants (G) of 293T cells infected with each rAAV viruses; wherein the GOI plasmid numbers corresponding to each plasmids are shown in Table 2, Mock/M: Blank control; RS1-HA: human RS1 protein with HA tag; NC: Non-treated cells.

FIG. 2: Comparison of the in vitro expression efficiency of RS1 after codon optimization, wherein FIG. 2A shows the structure for each GOI plasmid; FIG. 2B shows the expression of hRS1 in cells and supernatants of HEK293T cells infected with recombinant AAV; FIGS. 2C and 2D show the relative expression of RS1 of each plasmid in supernatants (C) and cell lysates (D);

FIG. 2E shows the correspondence between GOI plasmids and expression cassettes;

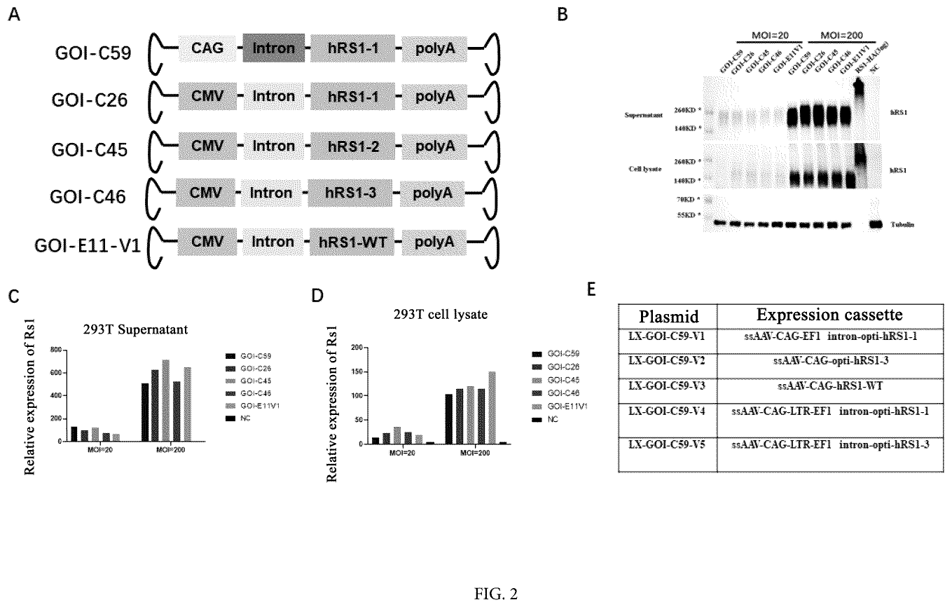

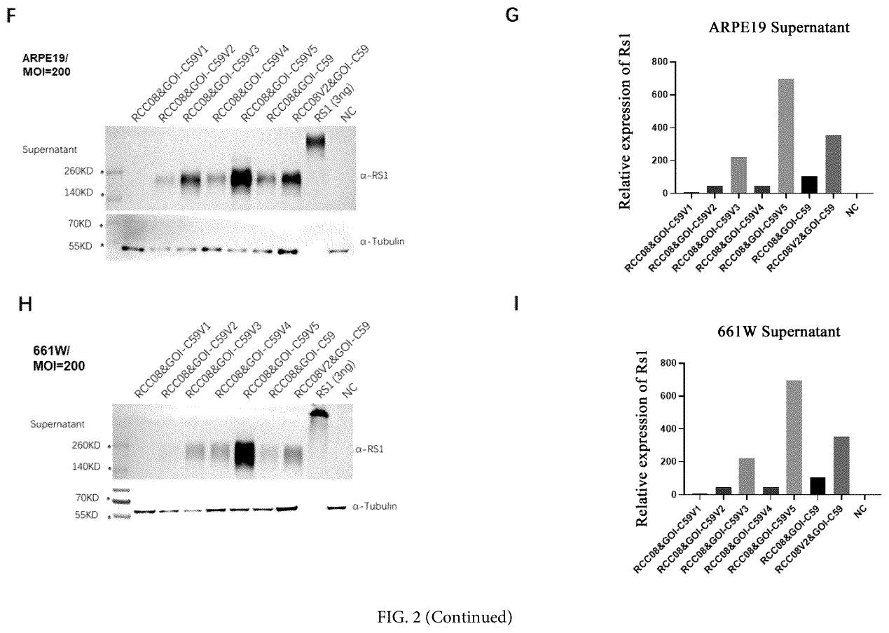

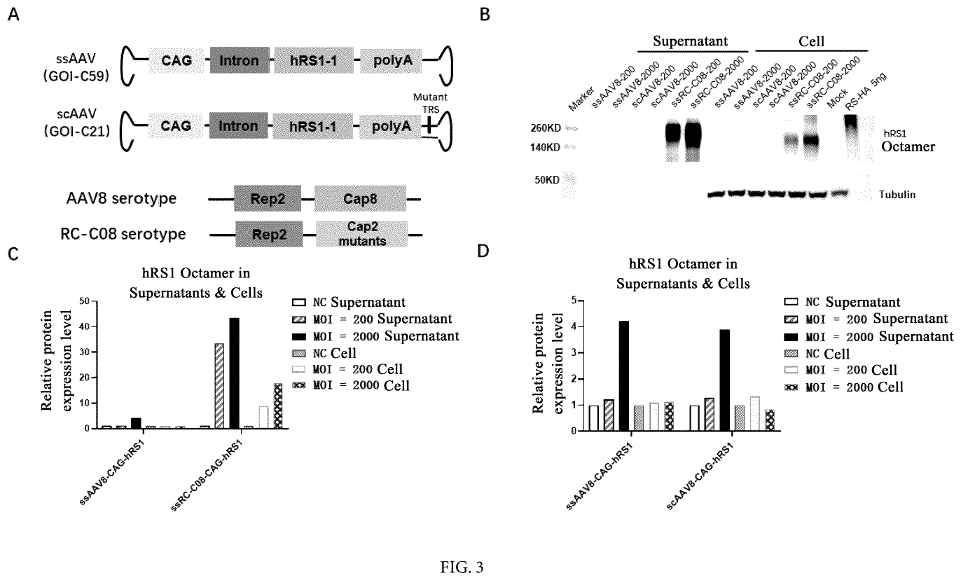

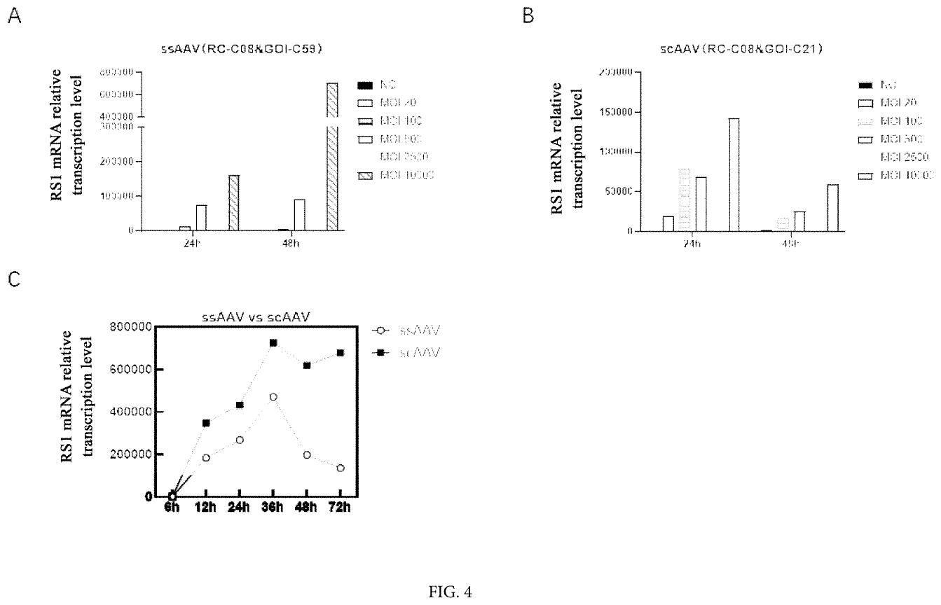

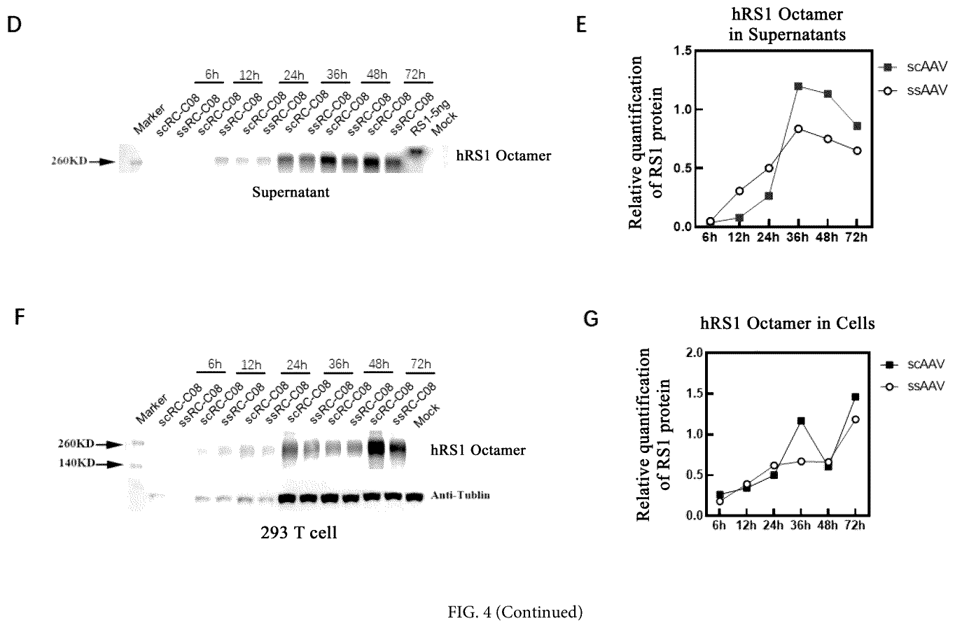

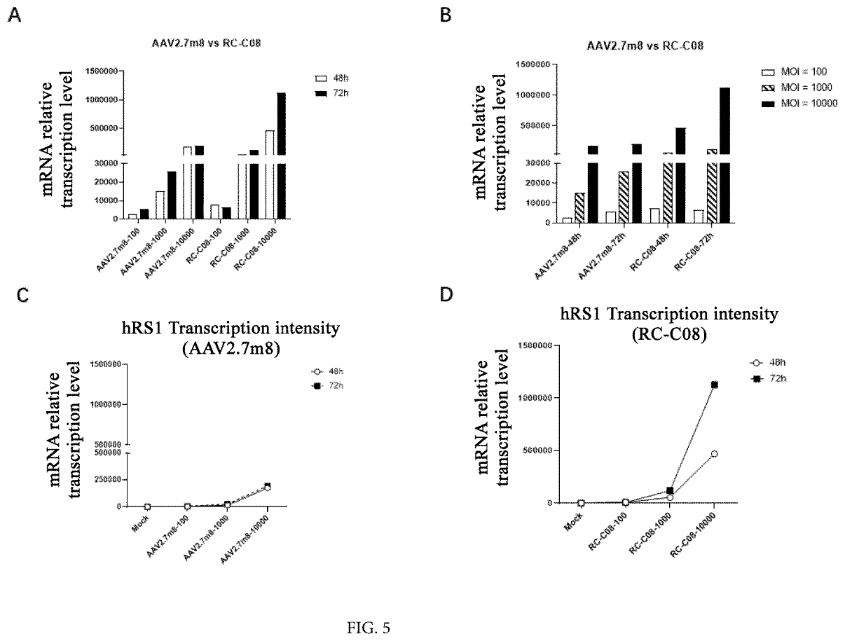

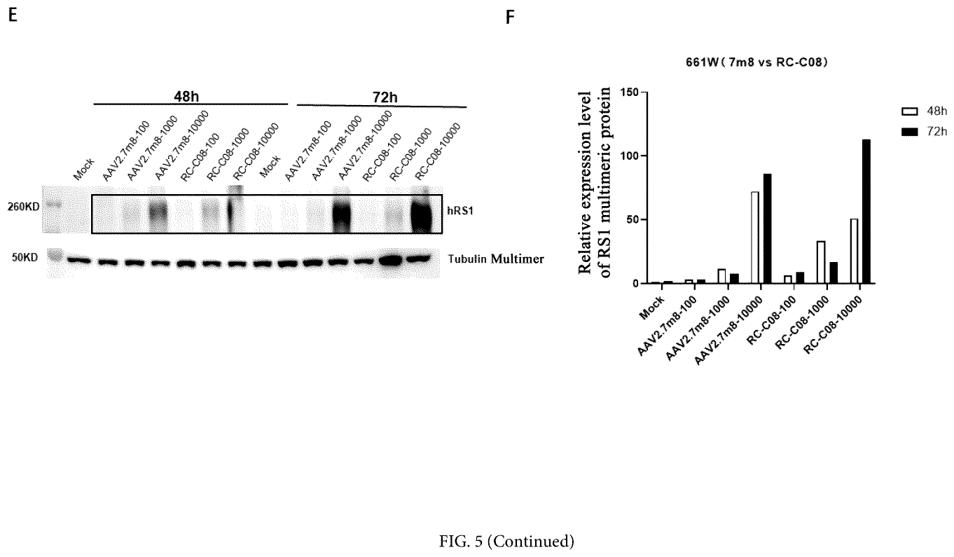

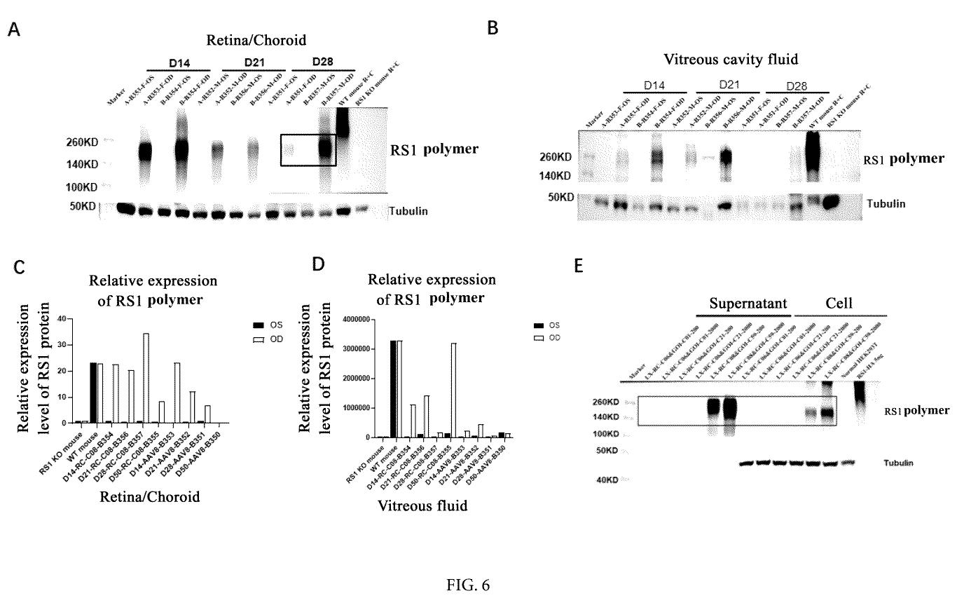

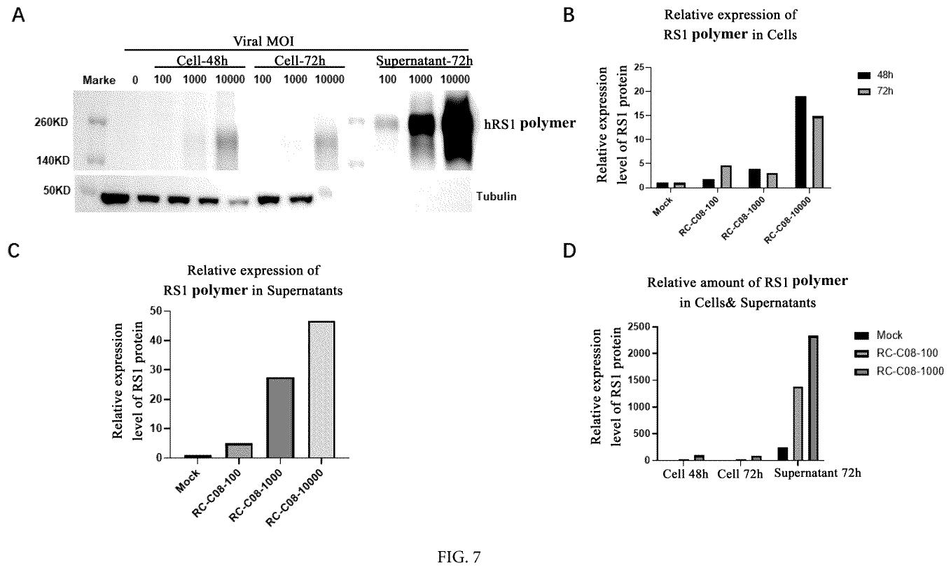

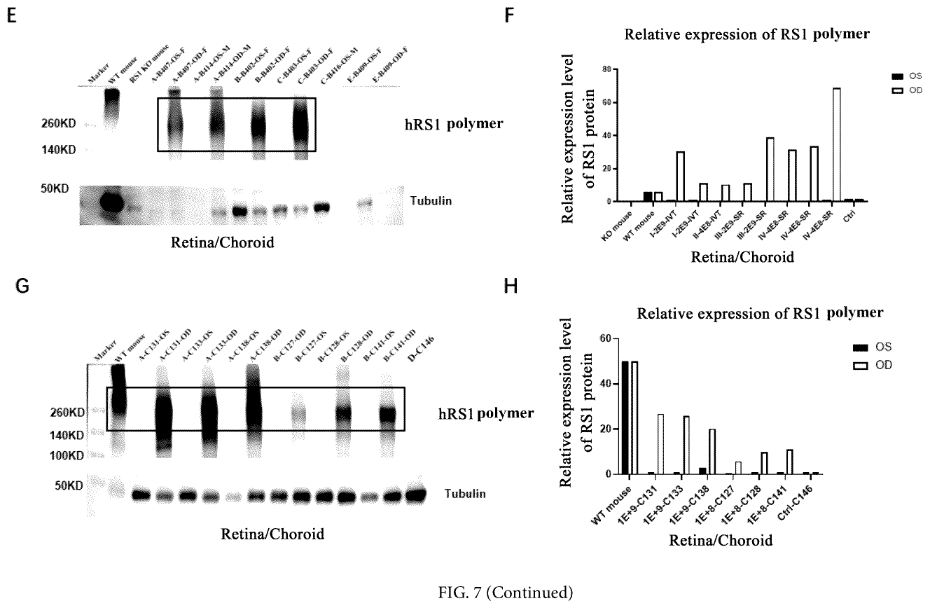

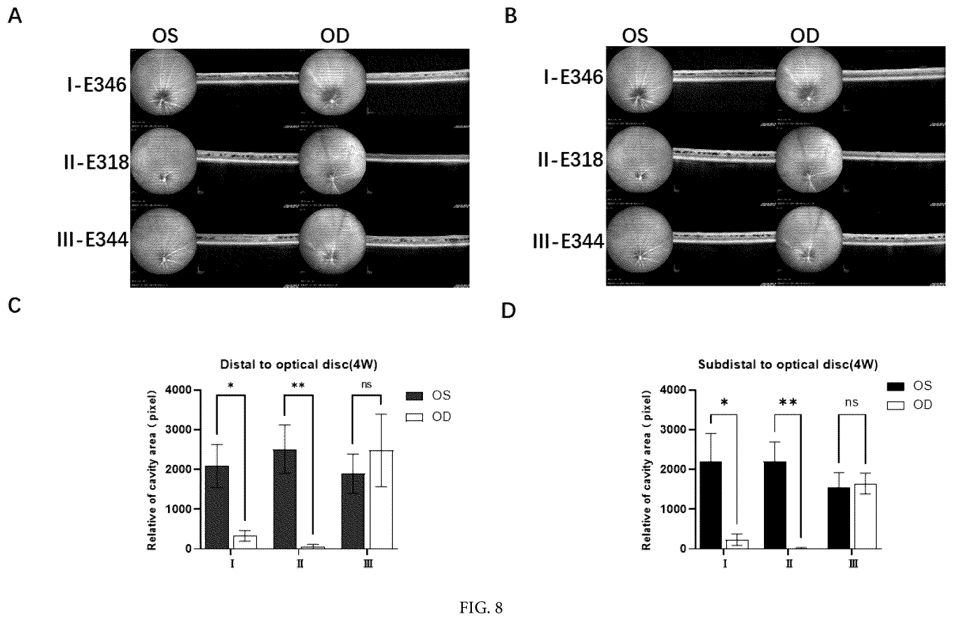

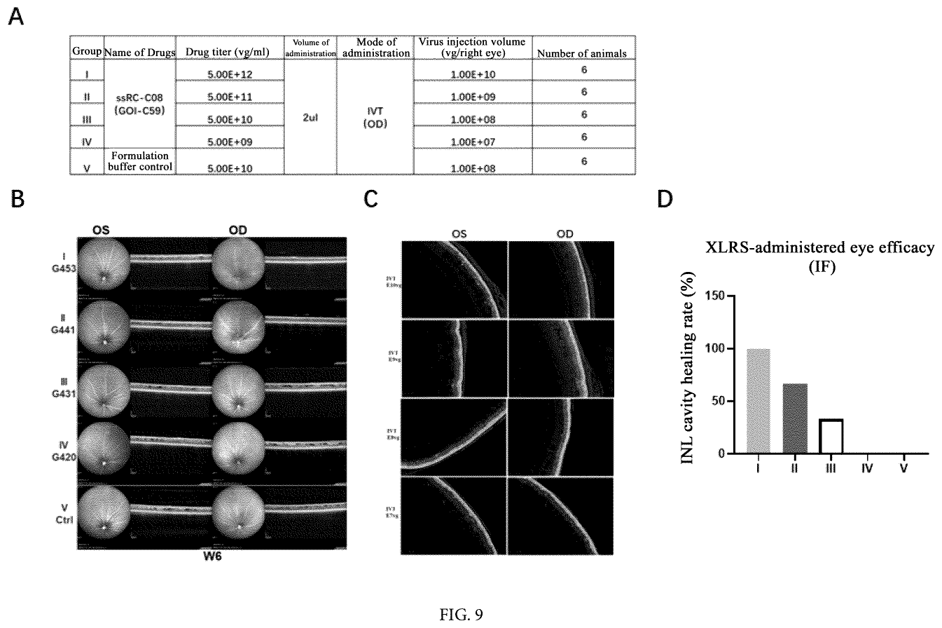

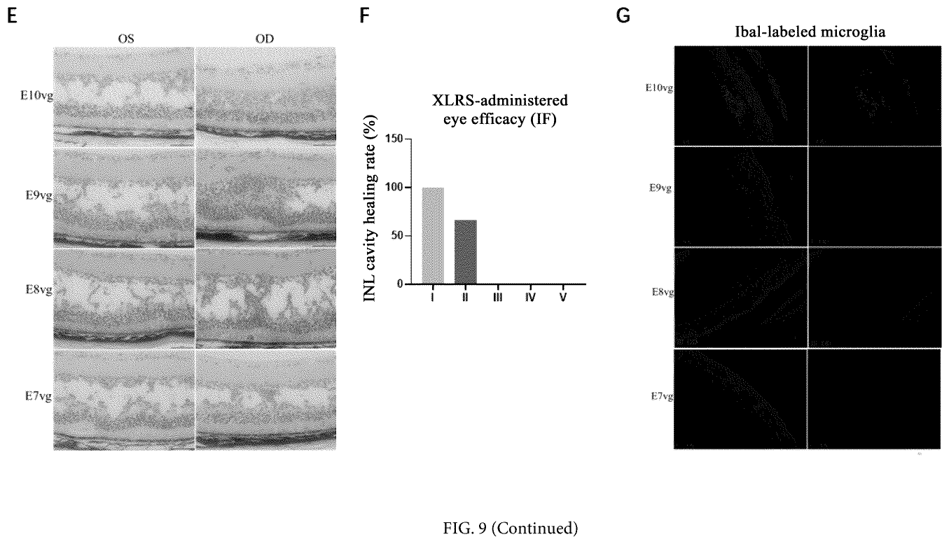

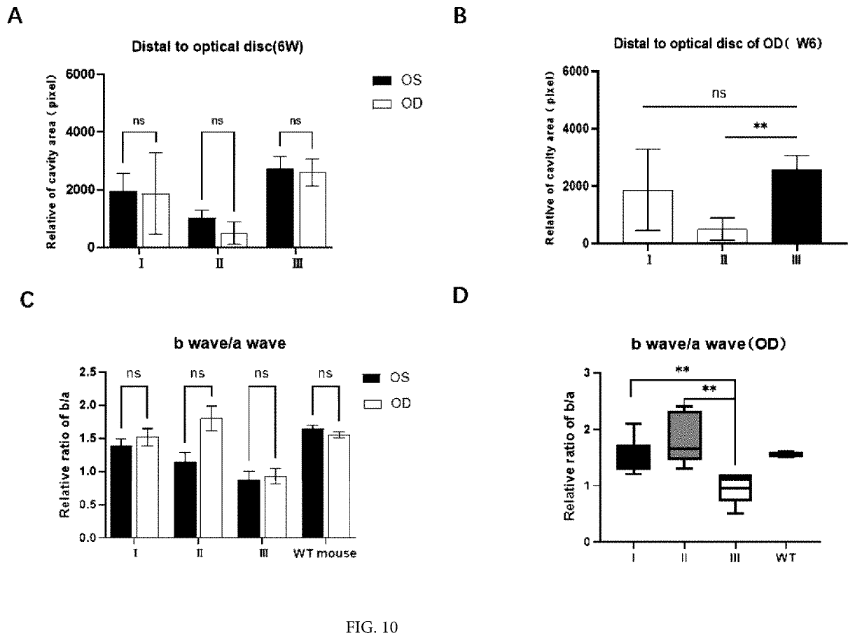

FIG. 2F shows the expression of hRS1 in the cell lysates and supernatants upon ARPE19 cell espression; FIG. 2G shows the relative expression of RS1 of each plasmid in the supernatants of APRE19 cells, FIG. 2H shows the expression of hRS1 in the cell lysates and supernatants upon 661W cell expression, and FIG. 2I shows the relative expression of RS1 of each plasmid in the supernatants of 661W cells;
wherein the GOI plasmid numbers corresponding to the each plasmid are shown in Table 2, NC represents non-treated cells.

FIG. 3: Comparison of hRS1 multimeric protein levels secreted in vitro by self-complementary AAV (scAAV) and single-stranded AAV (ssAAV), wherein FIG. 3A represents the structures of ssAAV and scAAV; FIG. 3B shows the WB image of the hRS1 octamer in supernatants and cells of HEK 293T expressed by respective packaging viruses; FIGS. 3C and 3D show the relative expression levels of hRS1 octamer in the supernatants and cells:
wherein Marker: a protein size indicator; ssAAV 8-200: single-stranded DNA (GOI-C59) & AAV8 serotype (multiplicity of infection of 200); scAAV 8-200: self-complementary DNA (GOI-C21) & AAV8 serotype (multiplicity of infection of 200), and so on, with the maximum multiplicity of infection MOI being 2000; Mock: Blank control; ssAAV8-CAG-hRS1: AAV8 serotype & GOI-C59; ssRC-C08-CAG-hRS1: RC-C08 serotype & GOI-C59; ssAAV8-CAG-hRS1: AAV8 serotype & GOI-C59; scAAV8-CAG-hRS1: AAV8 serotype & GOI-C21; NC supernatant: the supernatant of non-treated cells; NC cell: non-treated cell lysate.

FIG. 4: Comparison of interested protein transcription and protein expression levels by ssAAV and scAAV in 293T cells; FIG. 4A shows the relative transcriptional levels of RS1 mRNA in HEK293T cells infected with the RC-C08 capsid and GOI-C59 packaged virus (ssAAV) at different MOIs; FIG. 4B shows the relative transcriptional levels of RS1 mRNA in HEK293T cells infected with the RC-C08 capsid and GOI-C21 packaged virus (scAAV) at different MOIs; FIG. 4C shows the relative transcriptional levels of RS1 mRNA at different time points after infection of HKE293T cells with ssAAV and scAAV; FIGS. 4D and F show the expression of RS1 octamers in supernatant (D) and cells (F) at different time points as detected by nondenaturing WB after infection of HKE293T cells with ssAAV (also referred to as ssRC-C08) and scAAV (also referred to as scRC-C08); FIGS. 4E and G show the expression of RS1 octamer in supernatants (D) and cells (F) at different time points as detected by non-grayscale scanning statistics after infection of HKE293T cells with ssAAV and scAAV.

FIG. 5: Transcription levels and protein expression intensities of RS1 by ssRC-C08&GOI-C59 and ssAAV2.7m8&GOI-C59 in photoreceptor cells, wherein FIG. 5A shows the transcription levels of RS1 mRNA in 293T infected with viral particles prepared by AAV2.7m8 serotype capsid and RC-C08 serotype capsid with GOI-C59 at different MOIs (100, 1000, 10000), respectively; FIG. 5B shows a comparison of the transcriptional levels of RS1 mRNA at two time points, 48 h and 72 h, after infection of 293T with the two serotypes at different MOIs (100, 1000, 10000); FIG. 5C shows a dose-dependent curve of the transcriptional leveld of RS1 mRNA of ssAAV2.7m8&GOI-C59 versus MOI of the virus in 293T cells; FIG. 5D shows a dose-dependent curve of transcribed mRNA of the interested protein versus the MOI of the virus 48 h and 72 h after infecting cells with RC-C08&GOI-C59, respectively; FIG. 5E shows the expression of the monomer RS1 within 661W cells measured at 48 h and 72 h, respectively, after infection with the viral particles prepared by RC-C08 capsid and AAV2.7m8 capsid with GOI-C59;

FIG. 5F shows a histogram statistics of the data obtained from ImageJ grayscale scanning analysis of the WB photographed picture of FIG. 5E.

FIG. 6: Comparison of the expression intensities of interested proteins by ssRC-C08 and ssAAV8 in retinal tissue and vitreous humor in KO mice, wherein FIG. 6A shows the detection of RS1 protein in retinal tissue at different time points (Day 14/Day 21/Day 28) after administration of ssAAV8&GOI-C59 (group A OD administration to the right eye) and ssRC-C08&GOI-C59 (group B OD administration to right eye) (A or B for group A or group B, B353, etc. for the numbering of mice); FIG. 6B shows the detection of RS1 protein in vitreous humor within three weeks after administration for ssAAV8&GOI-C59 (group A OD administration to the right eye) and ssRC-C08&GOI-C59 (group B OD administration to the right eye); FIG. 6C shows ImageJ grayscale scanning analysis data of the western blot detection results of FIG. 6A; FIG. 6D shows ImageJ grayscale scanning analysis of the Western blot detection results of FIG. 6B; FIG. 6E shows a comparison (WB) of RS1 polymer expression by ssAAV8&GOI-C59 and ssRC-C08&GOI-C59 (two MOIs) in 293T.

FIG. 7: Study of RS1 protein expression following IVT and SR administration of ssrC-C08 & GOI-C59 in photoreceptor cells and model murine eye tissues. Wherein FIG. 7A shows the RS1 polymer expression by ssRC-C08&GOI-C59 in 661w cells; FIG. 7B shows a statistical analysis of RS1 polymer expression levels following in vitro transduction of cells with ssRC-C08&GOI-C59 (three gradient MOI); FIG. 7C shows a statistical comparison of the intensity of secreted RS1 polymer in supernatants after infection with ssRC-C08 &GOI-C59 at different multiplicity of infection (100, 1000, and 10000 MOI); FIG. 7D shows a statistical expression of RS1 polymer protein intracellularly and extracellularly secreted to the supernatant after transduction of 293T cells with ssRC-C08 &GOI-C59 at two multiplicity of infection at different time points; FIG. 7E shows the expression of RS1 polymer protein in Rs1 KO mice administered with two doses of ssRC-C08 & GOI-C59 in different ways (A-E represent mice in group A received IVT administration (2E9vg/OD); mice in group B received IVT administration (4E8vg/OD); mice in group C received subretinal administration (2E9vg/OD); mice in group D received subretinal administration (4E8vg/OD); mice in group E receiving no administration to the retina (control group); B407 represents the numbering of mice; OD represents the right eye; OS represents the left eye; M represents: Male; F represents: Female); FIG. 7F shows a grayscale scanning semi-quantitative statistics of 7E; FIG. 7G shows the expression of RS1 polymer protein in Rs1 KO mice by IVT administration of two doses of ssRC-C08&GOI-C59; FIG. 7. H shows a semi-quantitative statistical graph of the WB results of FIG. 7G after grayscale scanning.

FIG. 8: In vivo efficacy of ssRC-C08&GOI-C59 in the Rs1 knockout mice model, wherein FIGS. 8A and B show the retinal tissue morphology away from the optic disc in the model mice of drug candidates (two doses; IVT administration for 4 weeks) evaluated by OCT exam (E346, etc. are the numbering of mice); FIGS. 8A and B show the effect of drug candidates (ssRC-C08&GOI-C59) on retinal tissue morphology change near the optic disc in model mice evaluated by OCT tomography; FIG. 8C shows a grayscale scanning semi-quantitative data statistics of the cavitation area of FIG. 8A; FIG. 8D shows a grayscale scanning semi-quantitative bar chart of the cavitation area of FIG. 8B.

FIG. 9: Evaluation of dose effectiveness of ssrC-C08&GOI-C59 in Rs1 knockout model mice, wherein FIG. 9A shows a grouping and dosing schedule of drug candidates in a model mouse drug efficacy evaluation model; FIG. 9B shows the cavitation area in the retinal tissue of mice in each administration group evaluated by OCT tomography (I-IV); FIG. 9C shows a photograph of immunofluorescent staining (IF) of retinal tissue of mice in the administration group; FIG. 9D shows the healing of the retinal Inner Nuclear Layer (INL) cavitation in different groups of mice after treatment based on immunofluorescence photographs. FIG. 9E shows paraffin sections of the retinal tissue of mice in the I-IV (four doses, IVT) administration groups (HE staining); FIG. 9F shows a statistical bar graph of the healing of INL cavitation in Rs1 knockout model mice (HE, IVT); FIG. 9G shows a graph of immunofluorescent staining of microglia in retinal tissue from each administration group (IVT).

FIG. 10: Long-term efficacy assessment of candidate XLRS gene therapy drugs in RS1 knockout mouse model (OCT&ERG), FIG. 10A shows a semi-quantitative statistics of the area of cavitation distal to the optic disc in the left and right eyes (OS&OD) of model mice 6 weeks after administration (OCT exam); FIG. 10B shows a semi-quantitative statistics of the area of cavitation away from the optic disc in the administered eye (OD) in each administration group; FIG. 10C shows a statistics of the values from the ERG exam (b-wave/a-wave) in the left and right eyes of Rs1 KO model mouse six weeks after administration; FIG. 10D shows a statistics of the b-wave/a-wave values in the administered eyes of Rs1 KO mice six weeks after administration, wherein WT represents normal C57Balb/c mice.

### DETAILED DESCRIPTION OF THE INVENTION

### I. Recombinant AAV Viral Particle

The present invention relates to a recombinant AAV viral particle (rAAV) comprising
(i) an AAV capsid protein variant comprising an engineered capsid protein VP1 comprising the following amino acid mutation sites relative to a parental AAV capsid protein VP1: Q464V, A467P, D469N, 1470M, R471A, D472V, and S474G; or
an AAV capsid protein variant further comprising an engineered capsid protein VP1 comprising the following amino acid mutation sites relative to a parental AAV capsid protein VP1: Q464V, A467P, D469N, 1470M, R471A, D472V, S474G, and Y500F;
(iii) an interested nucleic acid packaged within the AAV capsid, the interested nucleic acid comprising a nucleic acid molecule encoding RS1.

### (I). AAV Capsid Protein Variant

Thus, the present invention also relates to an AAV capsid protein variant, i.e., a engineered capsid protein variant having multiple glycan binding sites.

In some embodiments, the AAV capsid protein variant comprises one or more amino acid substitutions, wherein the substitutions introduce new glycan binding sites into the AAV capsid protein. In some embodiments, the substitution introduces new glycan binding sites from a first AAV serotype into a capsid protein of a second AAV serotype that is different from the first AAV serotype.

In some embodiments, the AAV capsid protein variants suitable for the present invention comprise the following substitutions as compared to a parental capsid protein, e.g., the AAV capsid protein VP1 variants suitable for the present invention as compared to the parental AAV capsid protein VP1 comprise the following substitutions: Q464V, A467P, D469N, 1470M, R471A, D472V, and S474G, optionally said AAV capsid protein VP1 further comprises Y500F and/or S501A.

Parental capsid proteins such as parental capsid protein VP1 may be from a particular AAV serotype (AAV serotype 2 to AAV serotype 12) or a modified version of any of these serotypes (including AAV 4YF and AAV2.7m8 vectors), such as AAV serotype 1 (AAV1), AAV serotype 2 (AAV2), AAV2 modified version 7m8 (AAV2.7m8), AAV serotype 3a (AAV3a), AAV serotype 3b (AAV3b), AAV4 serotype 4 (AAV4), AAV serotype 5 (AAV5), AAV serotype 6 (AAV6), AAV serotype 7 (AAV7), AAV serotype 8 (AAV8), AAV serotype 9 (AAV 9), or AAV serotype 10 (AAV10).

In an exemplary embodiment, the parental capsid protein is a capsid protein of AAV2 or AAV2.7m8 serotype. In an exemplary embodiment, the parent capsid protein VP1 is capsid protein VP1 of AAV2 or AAV2.7m8 serotype. In an exemplary embodiment, the capsid protein VP1 of the parental AAV2 serotype comprises or consists of the amino acid sequence as set forth in SEQ ID NO: 51. In an exemplary embodiment, the capsid protein VP1 of the parental AAV2.7m8 serotype comprises or consists of an amino acid sequence as set forth in SEQ ID NO: 19.

In some embodiments, the galactose binding site of AAV9 may be introduced into the AAV2 parent.

In some embodiments, the AAV capsid protein variant of the present invention comprises the following substitutions as compared to the parental AAV2 or AAV2.7m8: Q464V, A467P, D469N, 1470M, R471A, D472V, and S474G, wherein the amino acid positions are determined with reference to the amino acid sequence of the AAV2.7m8 capsid protein (SEQ ID NO: 19).

In some embodiments, the AAV capsid protein variant of the present invention comprises the following substitutions as compared to the parental AAV2 or AAV2.7m8: Q464V, A467P, D469N, I470M, R471A, D472V, and S474G, wherein the amino acid positions are determined with reference to the amino acid sequence of the AAV2 capsid protein (SEQ ID NO: 51).

In some embodiments, the AAV capsid protein variant of the present invention
(i) comprises the amino acid sequence as set forth in SEQ ID NO:1;
(ii) comprises an amino acid sequence having at least 85%, 86%, 87%, 88%, 89%, 90%, 91%, 92%, 93%, 94%, 95%, 96%, 97%, 98% or 99% identity to the amino acid sequence as set forth in SEQ ID NO:1, and comprises the following substitutions: Q464V, A467P, D469N, 1470M, R471A, D472V, and S474G;
(iii) is encoded by the nucleic acid sequence as set forth in SEQ ID NO: 2; or
(iv) is encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:2, and comprises the following substitutions: Q464V, A467P, D469N, 1470M, R471A, D472V, and S474G.

In some embodiments, the present invention further relates to a nucleic acid comprising a nucleotide sequence encoding the capsid protein variant of the present invention.

In some embodiments, the nucleic acid of the present invention
(i) comprises or consists of the nucleic acid sequence as set forth in SEQ ID NO:2; or
(ii) comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:2, and the capsid protein variant encoded by the same comprises the following substitutions: Q464V, A467P, D469N, 1470M, R471A, D472V, and S474G.

The capsid protein variant of the present invention can contribute to increase the transduction activity of the virus particles, especially in the case of fewer mutations, the transduction activity (e.g., in vivo and/or in vitro transduction activity) of the virus particles can still be increased.

### (II) Interested Nucleic Acid

The interested nucleic acid comprised in the viral particles of the present invention, for example, encodes an RS1 protein, such as a human RS1 protein. In a preferred embodiment, the human RS1 protein
(i) comprises or consists of the amino acid sequence as set forth in SEQ ID NO:15; or
(ii) comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:15.

In one embodiment, wherein the human RS1 protein is encoded by an optimized RS1 gene coding sequence.

In one embodiment, the optimized RS1 gene coding sequence of the present invention comprises or consists of the nucleotide sequence as set forth in any one of SEQ ID NOs: 16-18 or a complementary sequence thereof.

In one embodiment, the optimized RS1 gene coding sequence of the present invention comprises a nucleotide sequence having at least 85%, 86%, 87%, 88%, 89%, 90%, 91%, 92%, 93%, 94%, 95%, 96%, 97%, 98%, or 99% identity to the nucleotide sequence as set forth in any one of SEQ ID NOs: 16-18 or a complementary sequence thereof, and encoding a human RS1 protein.

In one embodiment, the optimized RS1 gene coding sequence of the present invention can more efficiently express RS1 protein in mammalian (e.g., human) cells (e.g., photoreceptor cells, optic nerve cells) compared with the known RS1 gene coding sequence (e.g., the sequence as set forth in SEQ ID NO: 20).

The interested nucleic acid may be comprised in an expression cassette and thus packaged within the AAV capsid.

In some embodiments, the expression cassette comprises at least one ITR sequence (e.g., 1 or 2) flanking the interested nucleic acid, thereby allowing the vector genome to be packaged by the AAV capsid. The expression cassette may be single-stranded DNA, double-stranded DNA or single-stranded RNA or double-stranded RNA.

In some embodiments, the expression cassette may comprise one or more regulatory sequences to direct the expression of the RS1 gene coding sequence in a target cell (e.g., a retinal target cell such as a photoreceptor cell or an optic nerve cell). The regulatory sequence may be selected from the group consisting of a transcription initiation sequence, a termination sequence, a promoter, and a enhancer sequence operably linked to a coding sequence; an efficient RNA processing signals such as a splicing and polyadenylation (polyA) region, including a human growth hormone polyadenylation region; an inverted repeat sequence (e.g., L-ITR or R-ITR); a selectable marker or reporter gene, such as a resistance gene; a microRNA; a post-transcriptional regulatory sequence, such as WPRE (a woodchuck hepatitis virus post-transcriptional regulatory element); a sequence that stabilize cytoplasmic mRNA; a nucleic acid restriction site; a homologous recombination sequence; a sequence that enhances translation efficiency (e.g., Kozak consensus sequence); a sequence that enhances protein stability; and a sequence that enhances the secretion of the encoded product when desired.

In some preferred embodiments, the regulatory sequence is located in 5′UTR or 3′UTR. In some preferred embodiments, the regulatory sequence is selected from one or more of the group consisting of:
a promoter, an inverted repeat sequence, an intron, an enhancer, a post-transcriptional regulatory sequence, a polyadenylation region, a selectable marker, or a reporter gene.

Examples of promoters suitable for the present invention include, but are not limited to, the promoters from bacteria, yeast, plants, viruses, and mammals (including simians and humans). The promoter may be constitutive or may be inducible. Constitutive promoters initiate RNA synthesis independent of regulatory influences.

In a preferred embodiment of the present invention, the promoter is a CAG promoter which is a fusion of the cytomegalovirus (CMV) early enhancer and the chicken beta-actin promoter (e.g., via a 1-3 nucleotides linker, such as 1, 2 or 3 G).

In one embodiment, the chicken beta-actin promoter may comprise a nucleic acid sequence having at least about 90%, at least about 95%, at least about 96%, at least about 97%, at least about 98%, at least about 99% or more sequence identity to the nucleotide sequence as set forth in SEQ ID NO:6. In some embodiments, the chicken beta-actin promoter comprises or consists of the nucleotide sequence as set forth in SEQ ID NO: 6.

In one embodiment, the cytomegalovirus (CMV) early enhancer may comprise a nucleic acid sequence having at least about 90%, at least about 95%, at least about 96%, at least about 97%, at least about 98%, at least about 99% or more sequence identity to the nucleotide sequence as set forth in SEQ ID NO:5. In some embodiments, the promoter comprises or consists of the nucleotide sequence as set forth in SEQ ID NO: 5.

In one embodiment, the CAG promoter may comprise a nucleic acid sequence having at least about 90%, at least about 95%, at least about 96%, at least about 97%, at least about 98%, at least about 99% or more sequence identity to the nucleotide sequence as set forth in SEQ ID NO:32. In some embodiments, the promoter comprises or consists of the nucleotide sequence as set forth in SEQ ID NO: 32.

In one embodiment, the promoter is a CMV promoter. For example, the CMV promoter may comprise a nucleic acid sequence having at least about 90%, at least about 95%, at least about 96%, at least about 97%, at least about 98%, at least about 99%, or more sequence identity to the nucleotide sequence as set forth in SEQ ID NO:29. In some embodiments, the promoter comprises or consists of the nucleotide sequence as set forth in SEQ ID NO: 29.

The expression cassettes of the present invention may further comprise a selectable marker or a reporter gene, for example to determine the expression of the vector in a growth system (e.g., bacterial cells) or in target cells of the retina. The “selectable marker” or “reporter gene” of the present invention may be selected from those known in the art. Suitable reporter genes include, but are not limited to, an enhanced green fluorescent protein, a red fluorescent protein, luciferase, and secreted embryonic alkaline phosphatase (seAP), which may include sequences encoding geneticin, hygromycin, or puromycin resistance, and the like. Such selectable markers or reporter genes (which may or may not be located outside the viral genome to be packaged into viral particles) can be used to signal the presence of the plasmid in the bacterial cell, e.g., antibiotic resistance marker genes, such as ampicillin or tetracycline resistance or kanamycin resistance. In one embodiment, a selectable marker of the present invention is a kanamycin resistance marker, e.g., comprising a nucleic acid sequence having at least about 90%, at least about 95%, at least about 96%, at least about 97%, at least about 98%, at least about 99% or more sequence identity to the nucleotide sequence as set forth in SEQ ID NO:9. In some embodiments, the selectable marker comprises or consists of the nucleotide sequence as set forth in SEQ ID NO: 9.

A “post-transcriptional regulatory sequence” of the present invention is a DNA sequence that, when transcribed, enhances the expression of one or more transgenes or fragments thereof delivered by a viral vector of the present invention. The post-transcriptional regulatory sequence includes, but are not limited to, a hepatitis B virus post-transcriptional regulatory element (HPRE) and a woodchuck hepatitis virus post-transcriptional regulatory element (WPRE). The WPRE is a tripartite cis-acting element that has been shown to enhance transgene expression driven by certain, but not all, promoters.

The expression cassette or expression vector of the present invention may further comprise a polyadenylation region, such as the hGHpA (a human growth hormone polyadenylation region) or SV40 pA (a SV40 poly (A) signal polyadenylation region). In one embodiment, the polyadenylation region of the present invention comprises a nucleic acid sequence having at least about 90%, at least about 95%, at least about 96%, at least about 97%, at least about 98%, at least about 99% or more sequence identity to the nucleotide sequence as set forth in SEQ ID NO:8. In some embodiments, the polyadenylation region comprises or consists of the nucleotide sequence as set forth in SEQ ID NO: 8. In one embodiment, the polyadenylation region of the present invention comprises a nucleic acid sequence having at least about 90%, at least about 95%, at least about 96%, at least about 97%, at least about 98%, at least about 99% or more sequence identity to the nucleotide sequence as set forth in SEQ ID NO:12. In some embodiments, the polyadenylation region comprises or consists of the nucleotide sequence as set forth in SEQ ID NO: 12.

The expression cassette or expression vector of the present invention may also comprise an intron, such as a chimeric intron, for example derived from a Gallus gallus cytoplasmic beta-actin gene, with the sequence Gene ID of X00182.1. The use of chicken beta-actin intron 1 or functional equivalents thereof as an enhancer element or to express “hot spot” sequences has been disclosed in patents for the construction or recombination of mammalian expression vectors for the expression of recombinant proteins at a very high level, see CN200780052196.7 for details. Suitable introns for use in the expression cassettes of the present invention are known in the art. In some embodiments, the intron of the present invention comprises a nucleic acid sequence having at least about 90%, at least about 95%, at least about 96%, at least about 97%, at least about 98%, at least about 99% or more sequence identity to the nucleotide sequence as set forth in SEQ ID NOs: 7, 24, 28, or 31. In some embodiments, the intron sequence comprises or consists of the nucleotide sequence as set forth in SEQ ID NOs: 7, 24, 28, or 31 In some embodiments, the intron of the present invention comprises a nucleic acid sequence having at least about 90%, at least about 95%, at least about 96%, at least about 97%, at least about 98%, at least about 99% or more sequence identity to the nucleotide sequence as set forth in SEQ ID NO:7. In some embodiments, the intron sequence comprises or consists of the nucleotide sequence as set forth in SEQ ID NO: 7.

In one embodiment, the L-ITR of the present invention comprises a nucleic acid sequence having at least about 95%, at least about 96%, at least about 97%, at least about 98%, at least about 99% or more sequence identity to the nucleotide sequence as set forth in SEQ ID NO: 4 or 11. In some embodiments, the L-ITR comprises or consists of the nucleotide sequence as set forth in SEQ ID NO: 4 or 11. In one embodiment, the R-ITR of the present invention comprises a nucleic acid sequence having at least about 95%, at least about 96%, at least about 97%, at least about 98%, at least about 99% or more sequence identity to the nucleotide sequence as set forth in SEQ ID NO: 10 or 13. In some embodiments, the R-ITR comprises or consists of the nucleotide sequence as set forth in SEQ ID NO: 10 or 13.

In one embodiment, the L-ITR of the present invention comprises a nucleic acid sequence having at least about 95%, at least about 96%, at least about 97%, at least about 98%, at least about 99% or more sequence identity to the nucleotide sequence as set forth in SEQ ID NO: 4, and the R-ITR of the present invention comprises a nucleic acid sequence having at least about 95%, at least about 96%, at least about 97%, at least about 98%, at least about 99% or more sequence identity to the nucleotide sequence as set forth in SEQ ID NO: 10. In some embodiments, the L-ITR comprises or consists of the nucleic acid sequence as set forth in SEQ ID NO: 4, and the R-ITR comprises or consists of the nucleic acid sequence as set forth in SEQ ID NO: 10.

In one embodiment, the L-ITR of the present invention comprises a nucleic acid sequence having at least about 95%, at least about 96%, at least about 97%, at least about 98%, at least about 99% or more sequence identity to the nucleotide sequence as set forth in SEQ ID NO: 11, and the R-ITR comprises a nucleic acid sequence having at least about 95%, at least about 96%, at least about 97%, at least about 98%, at least about 99% or more sequence identity to the nucleotide sequence as set forth in SEQ ID NO: 13. In some embodiments, the L-ITR comprises or consists of the nucleic acid sequence as set forth in SEQ ID NO: 11, and the R-ITR comprises or consists of the nucleic acid sequence as set forth in SEQ ID NO: 13.

In one embodiment, the expression cassette of the present invention comprises, from 5′ to 3′, the following elements:

an L-ITR sequence, a CMV enhancer, a chicken beta-actin promoter sequence, a chimeric intron sequence, an optimized nucleic acid molecule of the present invention encoding RS1 protein, and downstream hGHpA sequence, R-ITR sequence.

In one embodiment, the expression cassette of the present invention comprises the nucleotide sequence shown in SEQ ID NO: 3 or 14, or consists of the nucleotide sequence, or comprises a nucleic acid sequence having at least about 80%, 85%, 90%, 95%, or at least about 96%, at least about 97%, at least about 98%, at least about 99% or more sequence identity to the nucleotide sequence as set forth in SEQ ID NO: 3 or 14.

### II. Preparation Method

The present invention relates to a method for preparing recombinant AAV viral particles (rAAV). Many methods are known in the art for packaging and producing rAAV. Currently, the commonly used rAAV packaging systems mainly include a triple-plasmid co-transfection system, a system with adenovirus as a helper virus, a packaging system with herpes simplex virus type 1 (HSV1) as a helper virus, and a baculovirus-based packaging system. Each packaging system has its own characteristics, and those skilled in the art can make appropriate selections according to needs.

The rAAV production cultures for production of rAAV viral particles all require: 1) suitable host cells including, for example, human-derived cell lines such as HEK-293T cells, or insect-derived cell lines (in the case of baculovirus production systems); 2) suitable helper virus functions provided by wild-type or mutant adenoviruses (e.g., temperature-sensitive adenoviruses), herpes viruses, baculoviruses or plasmid constructs providing helper functions; 3) AAV rep and cap genes and gene products; 4) genes of interest flanked by at least one AAV ITR sequence, and preferably driven by an operably linked promoter; and 5) suitable culture systems to support rAAV production.

In some embodiments, the present invention relates to a method of producing a recombinant AAV viral particle, comprising culturing a packaging cell under conditions sufficient for production of the recombinant AAV viral particle, wherein the packaging cell comprises a plasmid comprising a nucleic acid encoding the capsid protein variant according to the present invention or the capsid protein variant coding nucleic acid of the present invention.

In some embodiments, the packaging cell further comprises a helper plasmid and/or a transfer plasmid comprising the interested nucleic acid.

In some embodiments, the method further comprises isolating from the culture supernatant a self-complementary recombinant AAV viral particle.

In some embodiments, the method further comprises lysing the packaging cell, and isolating a recombinant AAV viral particle from the cell lysate.

In some embodiments, the method further includes one or more of the following steps:
a. removing the cell debris,
b. treating the supernatant containing the recombinant AAV viral particle with a benzonase nuclease,
c. concentrating the recombinant AAV viral particle,
d. purifying the recombinant AAV viral particle.

Thus, the present invention also relates to a packaging cell for producing a recombinant AAV viral particle, said packaging cell comprising a plasmid comprising a nucleic acid encoding the capsid protein variant of the present invention or the capsid protein coding nucleic acid of the present invention or the expression cassette of the present invention.

### III Composition, Medicament or Formulation

The present invention provides a formulation, composition or medicament, which contains (a) the rAAV of the present invention, and (b) pharmaceutically acceptable auxiliary material, such as pharmaceutically acceptable carriers and excipients known in the art, including buffers.

As used herein, “pharmaceutically acceptable carrier” includes any and all solvents, dispersion medium, isotonic agent and absorption delaying agents, and the like that are physiologically compatible. See also “Handbook of Pharmaceutical Excipients”, 8th Edition, R. C. Rowe, P. J. Seskey and S. C. Owen, Pharmaceutical Press, London, Chicago for the use of pharmaceutically acceptable auxiliary material and uses thereof.

In some embodiments, the pharmaceutically acceptable auxiliary material include, but are not limited to, one or more compatible solid or liquid fillers or gel materials that are suitable for human use and must be of sufficient purity and sufficiently low toxicity. By “compatible” is meant herein that the components of the composition are capable of admixing with and between the active ingredients of the present invention without significantly diminishing the efficacy of the active ingredients. Suitable pharmaceutically acceptable auxiliary material will be known to those skilled in the art. Examples of pharmaceutically acceptable carrier moieties are cellulose and its derivatives (e.g., sodium carboxymethylcellulose, sodium ethylcellulose, cellulose acetate, etc.), gelatin, talc, solid lubricants (e.g., stearic acid, magnesium stearate), calcium sulfate, vegetable oils (e.g., soybean oil, sesame oil, peanut oil, olive oil, etc.), polyols (e.g., propylene glycol, glycerin, mannitol, sorbitol, etc.), emulsifiers (e.g., Tween®), wetting agents (e.g., sodium lauryl sulfate), colorants, flavors, stabilizers, antioxidants, preservatives, pyrogen-free water, and the like.

The formulation or composition or medicament of the present invention may be a liquid or a solid, such as a powder, gel or paste. Preferably, the formulation or composition or medicament of the present invention is a liquid, preferably an injectable liquid. Preferably, the injectable liquid is provided as a capsule or in a syringe.

The rAAV of the present invention, or a formulation, composition, or medicament comprising the same, may be administered intravenously, intramuscularly, subcutaneously, orally, by mucosal contact, intraperitoneally, and intralesionally, preferably topically to the eye, for example by intraretinal or intravitreal administration, such as intravitreal injection, subretinal injection, or suprachoroidal injection. In some embodiments, the rAAV of the present invention, or a formulation or composition or medicament comprising the same, may be administered intraretinally or intravitreally, e.g., by intravitreal or subretinal administration, e.g., intravitreal administration (IVT administration). In either mode of administration, preferably, the formulation or composition or medicament of the present invention is provided as an injectable liquid.

The composition or formulation or medicament may comprise physiologically acceptable sterile aqueous or anhydrous solutions, dispersions, suspensions or emulsions, and sterile powders for reconstitution into sterile injectable solutions or dispersions. Suitable aqueous and nonaqueous carriers, diluents, solvents or excipients include water, ethanol, polyols and suitable mixtures thereof.

The composition, e.g., pharmaceutical composition or pharmaceutical formulation, of the present invention may further comprise additional active ingredients, e.g., one or more additional therapeutic agents, e.g., an immunomodulator, e.g., an immunosuppressant.

### IV. Combination Product

In one aspect, the present invention also provides a combination product (e.g., a pharmaceutical combination product) comprising the rAAV of the present invention, and one or more additional therapeutic agents. The combination product of the present invention may be used in the therapeutic method of the present invention.

The present invention also provides a pharmaceutical kit comprising the combination product, e.g. the pharmaceutical kit comprises the following in the same package:
a first container containing the rAAV of the present invention or a medicament comprising said rAAV;
a second container containing a pharmaceutical composition comprising one or more additional therapeutic agents, e.g., an immunomodulator.

In some embodiments, the additional therapeutic agent is an immunomodulator, e.g., an immunosuppressant, e.g., for reducing an immune response, such as an immunoinflammatory response, generated by the rAAV particle.

### V. Therapeutic Method

In one embodiment, the rAAV, the formulation or the composition or the medicament of the present invention is used for the treatment of ocular diseases, including but not limited to, diseases caused by retinopathy or chorioretinopathy, age-related macular degeneration, diabetic retinopathy, and other non-hereditary ophthalmic diseases such as physically or chemically induced retinal damage.

In another preferred embodiment, the rAAV, formulation or composition or medicament of the present invention is used for the treatment of X-linked retinoschisis.

In another preferred embodiment, the rAAV, formulation or composition or medicament of the present invention is used for the treatment of retinitis pigmentosa.

In one embodiment, the rAAV, formulation or composition or medicament of the present invention is administered intraocularly, e.g., via intraretinal or intravitreal administration, e.g., via subretinal or intravitreal administration (IVT administration). In one embodiment, the administration is injection.

In one embodiment, the rAAV of the present invention is administered at a dose of greater than or equal to about 10³vg/eye/dose, 10⁴vg/eye/dose, 10⁵vg/eye/dose, 10⁶vg/eye/dose, 10⁷vg/eye/dose, 10⁸vg/eye/dose, 10⁹vg/eye/dose, 10¹⁰ vg/eye/dose, 10¹¹vg/eye/dose, or 10¹²vg/eye/dose, preferably no more than 10¹²vg/eye/dose, for example, greater than or equal to 2×10⁸vg/eye/dose, 3×10⁸vg/eye/dose, 4×10⁸vg/eye/dose or 5×10⁸vg/eye/dose, for example between about 10⁷vg/eye/dose and about 5×10⁸vg/eye/dose, between 1×10⁷vg/eye/dose and about 5×10⁸vg/eye/dose, for example, about 2×10⁸vg/eye/dose, 3×10⁸vg/eye/dose, 4×10⁸vg/eye/dose or 5×10⁸vg/eye/dose.

In one embodiment, for intravitreal administration, the dose of a rAAV of the present invention is greater than or equal to about 10⁶vg/eye/dose, 10⁷vg/eye/dose, 10⁸vg/eye/dose, 10⁹vg/eye/dose, 10¹⁰ vg/eye/dose, 10¹¹vg/eye/dose, or 10¹²vg/eye/dose, preferably no more than 10¹²vg/eye/dose, for example greater than or equal to 2×10⁸vg/eye/dose, 3×10⁸vg/eye/dose, 4×10⁸vg/eye/dose or 5×10⁸vg/eye/dose, for example, between about 10⁷vg/eye/dose and about 5×10⁸vg/eye/dose, between 1×10⁷vg/eye/dose and about 5×10⁸vg/eye/dose, for example, about 2×10⁸vg/eye/dose, 3×10⁸vg/eye/dose, 4×10⁸vg/eye/dose or 5×10⁸vg/eye/dose.

In one embodiment, the present invention also relates to the use of a recombinant AAV viral particle, a formulation or composition or a combination product comprising the same, in the manufacture of a medicament for the treatment of an ocular disease according to the present invention.

### VI. Definitions

For purposes of interpreting this specification, the following definitions will be used, and where appropriate, terms used in the singular may also include the plural, and vice versa. Unless otherwise specified, all technical and scientific terms used herein have the same meanings as those generally understood by a person of ordinary skill in the art to which the present invention belongs.

The term “about” or “approximately” includes statistically significant ranges of values. Such ranges may be within an order of magnitude of a given value or range, preferably within 50%, more preferably within 20%, still more preferably within 10%, and even more preferably within 5% or within 1%. The allowable variations covered by the terms “about” or “approximately” depend on the particular system under study and can be readily appreciated by one of ordinary skill in the art.

As used herein, the term “and/or” means any one of the options or two or more or all of the options.

As used herein, the terms “comprising” or “including” means including the recited elements, integers, or steps, but not excluding any other elements, integers, or steps. When the term “comprising” or “including” is used herein, unless otherwise indicated, it also encompasses the situation consisting of the stated elements, integers, or steps. For example, reference to an antibody variable region “comprising” a particular sequence is also intended to encompass antibody variable regions consisting of that particular sequence.

As used herein, “RS1 gene”, “RS1”, “XLRS1” are used interchangeably herein and refer to the congenital retinoschisis gene. The RS1 gene (retinoschisin 1) is the causative gene of XLRS, which is located at Xp22 in humans, consisting of 6 exons, with Genbank accession number of NC_000023. The expression product encoded by the RS1 gene is called “RS1 protein” and consists of 224 amino acids. The protein has a discoidin domain (encoded by exons 4-6) that is present in a large number of families of secreted or membrane-bound proteins. The RS1 protein is involved in cell adhesion and cell interaction, and is highly conserved among species. RS1 protein is secreted mainly in the outer layer of the retina, and its loss results in retinal cavitation, synaptic dysfunction, visual deterioration, and easy detachment of the retina. In one embodiment, the RS1 protein is human RS1 protein. RS1 proteins described herein also encompass natural variants thereof. In a preferred embodiment, the RS1 protein comprises the amino acid sequence as set forth in SEQ ID NO:15 or comprises an amino acid sequence having at least 95%, 96%, 97%, 98%, 99%, or 100% identity to the amino acid sequence as set forth in SEQ ID NO:15.

As described herein, adeno-associated virus (AAV), also known as adeno-associated virus, belongs to the genus Dependovirus in the family Parvoviridae, and is a single-stranded DNA-deficient virus with the simplest structure that is currently discovered, requiring a helper virus (usually an adenovirus) to participate in replication. It encodes the cap gene and the rep gene in inverted repeats (ITRs) at both ends. ITRs are crucial for the replication and packaging of viruses. The cap gene encodes the capsid protein of the virus, and the rep gene is involved in the replication and integration of the virus. AAV can infect a variety of cells. As the adeno-associated virus is smaller than other virus vectors and has no pathogenicity, and can transfect dividing and non-dividing cells, the gene therapy method based on the AAV vector for ocular diseases, especially for inherited retinal degeneration, has attracted extensive attention. The recombinant adeno-associated virus (rAAV) is derived from non-pathogenic wild type adeno-associated virus, is considered to be one of the most promising gene transfer vectors due to the characteristics of good safety, wide host cell range (dividing and non-dividing cells), low immunogenicity, long time for expressing foreign genes in vivo and the like, and is widely applied to gene therapy and vaccine research in the world. Over 10 years of research, the biological properties of recombinant adeno-associated virus (rAAV) have been well understood, and many data have been accumulated on the effects of rAAV in various cells, tissues, and in vivo experiments. In medical research, rAAV is used in the study of gene therapy for a variety of diseases (including in vivo, in vitro experiments); meanwhile, the gene transfer vector is used as a characteristic gene transfer vector and is widely applied to the aspects such as gene function research, disease model construction, and gene knock-out mouse preparation.

The term “capsid protein” includes proteins that are part of the viral capsid. For adeno-associated viruses, the capsid proteins are commonly referred to as VP1, VP2, and/or VP3, and are each encoded by a single cap gene. For AAV, these three AAV capsid proteins are produced in an overlapping fashion from the cap Open Reading Frame (ORF) by using alternative mRNA splicing and/or alternative translation start codon usage, although all three proteins use a common stop codon. Warrington et al., (2004) J. virol. 78:6595 is incorporated herein by reference in its entirety. VP1 of AAV2 is generally translated from the ATG start codon (amino acid M1) on 2.4-kb mRNA, while VP2 and VP3 of AAV2 originate from the smaller 2.3-kb mRNA, using the weaker ACG start codon for VP2 (amino acid T138) and read-through translation to the next available ATG codon (amino acid M203) for the most abundant capsid protein VP3. The amino acid sequence of the capsid proteins of adeno-associated viruses is well known in the art and is generally conserved, in particular depending on parvovirus. See Rutledge et al., (1998) J. Virol. 72:309-19. Accordingly, although the amino acid positions provided herein may be provided with respect to the VP1 capsid protein of AAV, and as not specifically indicated, the amino acid positions provided herein are determined with reference to the amino acid position of AAV2 VP1 as set forth in SEQ ID NO:51. Those skilled person will be able to determine individually and readily the position of the same amino acid within the VP2 and/or VP3 capsid protein of AAV, and the corresponding position of the amino acid in the different serotypes. In addition, the skilled person is able to make corresponding mutations between capsid proteins of different AAV serotypes for the formation of “capsid protein variants”.

As used herein, the phrase “operably linked” includes physical juxtaposition of components or elements (e.g., in three-dimensional space) that directly or indirectly interact with each other, or otherwise coordinate with each other to participate in a biological event, the juxtaposition achieving or allowing such interaction and/or coordination. In some embodiments, “operably linked” refers to the covalent attachment of related components or elements to each other. However, those skilled in the art will appreciate that in some embodiments, covalent attachment is not required to achieve effective operably attachment.

The term “capsid protein variant” includes the capsid proteins having at least one mutation (e.g., substitution, deletion or insertion) as compared to the corresponding capsid protein as a parent.

The terms “transduction” or “infection” and the like refer to the introduction of a nucleic acid into a target cell by a viral vector. The term “transduction efficiency” refers to the fraction (e.g., percentage) of cells that express the interested nucleotide after incubation with a set number of viral vectors containing the interested nucleotide. Well-known methods for determining transduction efficiency include fluorescence activated cell sorting of cells transduced with a fluorescent reporter gene, PCR for expression of the interested nucleotide, and the like.

“Percent (%) identity” of an amino acid sequence or nucleic acid sequence refers to the percentage of amino acid residues/nucleotides in the candidate sequence that are identical to the amino acid residues/nucleotides of the specific sequence shown in the specification, after aligning the candidate sequence with the specific sequence shown in the specification and introducing gaps, if necessary, to achieve the maximum percent sequence identity, and not considering any conservative substitutions as part of the sequence identity. In some embodiments, the present invention contemplates variants of a protein or polypeptide or nucleic acid of the present invention that have a substantial degree of identity, e.g., at least 80%, 85%, 90%, 95%, 97%, 98%, or 99% or more identity, relative to the polypeptide or protein or nucleic acid specifically disclosed herein. The variant may comprise conservative changes.

The terms “individual” or “subject” are used interchangeably herein to refer to mammals. Mammals include, but are not limited to, domesticated animals (e.g., cows, sheep, cats, dogs, and horses), primates (e.g., humans and non-human primates such as monkeys), rabbits, and rodents (e.g., mice and rats). In particular, the individual is a human.

The term “treating” includes administering the composition or hybrid polypeptide to prevent or delay the onset of the symptoms, complications, or biochemical indicators of a disease, alleviate the symptoms, or arrest or inhibit the further development of the disease, condition, or disorder. The term “preventing” includes the inhibition of the occurrence or development of a disease or disorder or the symptoms of a particular disease or disorder.

The term “pharmaceutically acceptable auxiliary material” refers to a diluent, adjuvant (e.g., Freund's adjuvant (complete and incomplete)), excipient, carrier or stabilizer, etc., with which the active substance is administered.

The term “pharmaceutical composition” refers to a composition that is present in a form that allows the biological activity of the active ingredients contained therein to be effective, and which does not contain additional ingredients having unacceptable toxicity to the subject to which the composition is administered.

The term “effective amount” refers to an amount or dose of a hybrid protein or a nucleic acid encoding the same or composition or combination of the present invention which, upon administration to a patient in a single or multiple dose, produces the desired effect in the patient in need of treatment or prevention.

A “therapeutically effective amount” refers to an amount effective, at dosages and for periods of time required, to achieve the desired therapeutic result. A therapeutically effective amount is also one in which any toxic or adverse effect of the hybrid protein or composition or combination is less than a therapeutically beneficial effect. A “therapeutically effective amount” preferably inhibits or improve a measurable parameter by at least about 40%, even more preferably at least about 50%, 55%, 60%, 65%, 70%, 75%, 80%, 85%, 90% or even 100% relative to untreated subjects.

A “prophylactically effective amount” refers to an amount effective, at dosages and for periods of time required, to achieve the desired prophylactic result. Generally, a prophylactically effective amount will be less than a therapeutically effective amount because a prophylactic dose is used in a subject prior to or at an earlier stage of the disease.

Any documents cited herein, including patents, patent applications, and literature, are incorporated herein in their entirety.

Any or all of the features discussed above and throughout this invention may be combined in various embodiments of the present invention. The following examples further illustrate the present invention, however, it should be understood that the examples have been described by way of illustration and not limitation, and are not intended to, and should not be construed to, limit the scope of the present invention in any way, and that various modifications may be made by those skilled in the art.

### EXAMPLE

### Example 1: Construction of GOI Plasmids and Comparison of Expression Efficiency at the Cellular Level for Different Expression Cassettes

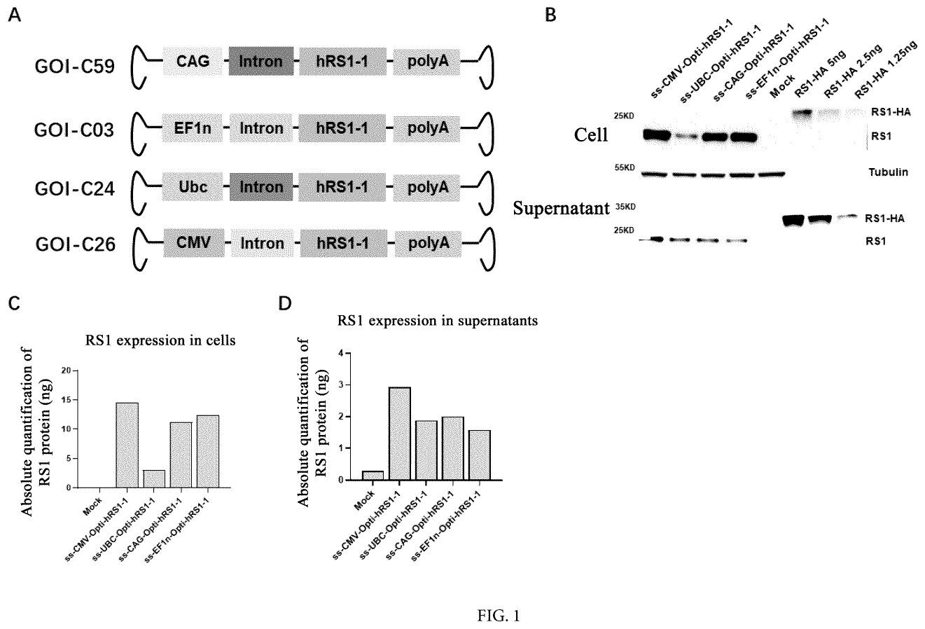

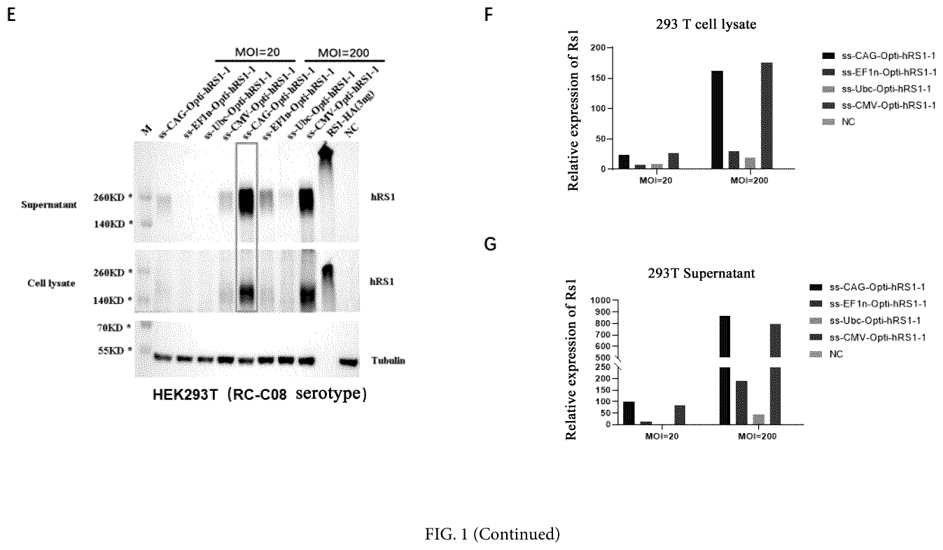

Cloning process of GOI plasmid: first, GOI-E04 (see SEQ ID NO: 21 for sequence) was synthesized by using a gene synthesis technology as a basic vector, which was linearized using NotI (nt position 143) at the N-terminal, and Xhol (nt position 3243) at the C-terminal; CAG-Intron-hRS1-1 (CAG-Intron template from GOI-E04 plasmid, CAG promoter: SEQ ID NO: 32), EFIn-Intron-hRS1-1 (EFIn promoter: SEQ ID NO:22; Intron: SEQ ID NO:24; EFIn-Intron: SEQ ID NO:23; hRS1-1: SEQ ID NO:16), Ubc-Intron-hRS1-1 (Ubc promoter: SEQ ID NO: 26; Intron: SEQ ID NO:28; Ubc-Intron: SEQ ID NO:27; hRS1-1: SEQ ID NO:16), CMV-Intron-hRS1-1 sequence (CMV promoter: SEQ ID NO:29; Intron: SEQ ID NO:31; CMV-Intron: SEQ ID NO:30; hRS1-1: SEQ ID NO:16) of different promoter elements were enriched by PCR, and then subjected to homologous recombination to replace the GOI-E04 nt 143-3243 sequence, resulting in clones of GOI-C59, GOI-C03, GOI-C24, GOI-C26, as shown in FIG. 1A, with primer sequences as shown in Table 1.

GOI-C26 was used as a basic vector, which was linearized using EcoRI (nt position 1323) at the N-terminal, and BamHI (nt position 2010) at the C-terminal; different hRS1 CDS (hRS1-2 (SEQ ID NO:17), hRS1-3 (SEQ ID NO:18), hRS1-WT (SEQ ID NO:20)) sequences were amplified and enriched by PCR, and then subjected to homologous recombination to replace GOI-C26 nt 2030-2749 sequences, resulting in clones of GOI-C45, GOI-C46, and GOI-E11-V1, respectively, with the primer sequences shown in Table 1.

GOI-C59 was used as a basic vector, which was linearized using BglII (nt position 3226) at the N-terminal and FspI (nt position 3518) at the C-terminal, a synthesized 3′-terminal ITR TRS binding site deletion fragment as a template was enriched by PCR, and then subjected to homologous recombination to replace GOI-C59 nt 3226-3518 sequences, resulting in a clone of GOI-C21, wherein the skeleton structure of the clone is scAAV, with the primer sequences shown in Tables 1 and 2.

TABLE 1
PCR primer information

| | SEQ | |
|---|---|---|
| | ID | |
| Clone | NO | Primers |
| GOI-C03 | 33 | forward: |
| | | 5′-actaggggttcctgcggc |
| | | cgcacgcgtggaattcgctagtta-3′ |
| | 34 | reverse: |
| | | 5′-gccacccgtagatctacg |
| | | gtatcatatcgataag-3′ |
| GOI-C24 | 35 | forward: |
| | | 5′-actaggggttcctgcggc |
| | | cgcggcctccgcgccgggttttg-3′ |
| | 36 | reverse: |
| | | 5′-gccacccgtagatctctc |
| | | gagttaggcgcacttgctcacgc-3′ |
| GOI-C26 | 37 | forward: |
| | | 5′-actaggggttcctgcggc |
| | | cgcacgcgtctagttattaatag-3′ |
| | 38 | reverse: |
| | | 5′-ccacccgtagatctctcg |
| | | agcagcgctgctcgaggcaagc-3′ |
| GOI-C59 | 39 | forward: |
| | | 5′-tcactaggggttcctatc |
| | | gatatcaagcttcgttac-3′ |
| | 40 | reverse: |
| | | 5′-agggatgccacccgtgga |
| | | tcctcaggcgcacttg-3′ |
| GOI-C45 | 41 | forward: |
| | | 5′-ttcgaacatcgattgaat |
| | | tctgagccaccatgtccaggaaaatc-3′ |
| | 42 | reverse: |
| | | 5′-aggtcgactctagaggat |
| | | cctcaggcgcacttgctcacac-3′ |
| GOI-C46 | 43 | forward: |
| | | 5′-ttcgaacatcgattgaat |
| | | tctgagccaccatgtacagaatg-3′ |
| | 44 | reverse: |
| | | 5′-aggtcgactctagaggat |
| | | cctcaggcacacttactcacac-3′ |
| GOI-E11- | 45 | forward: |
| | | 5′-gactctagaggatcgaat |
| | | tcgccaccatgtcacgcaagatag-3′ |
| V1 | 46 | reverse: |
| | | 5′-gggatgccacccgtggat |
| | | cctcaggcacacttgctgacg-3′ |
| GOI-C21 | 47 | forward: |
| | | 5′-caagttaacaacaacaat |
| | | tgcattcattttatgtttcag-3′ |
| | 48 | reverse: |
| | | 5′-acggccagtgaattgtcg |
| | | acgcatgcgggcccggatctg-3′ |

TABLE 2
GOI plasmid information

| Plasmid name | expression cassette |
|---|---|
| GOI-C59 | ss-CAG-Opti-hRS1-1 |
| GOI-C03 | ss-EF1n-Opti-hRS1-1 |
| GOI-C24 | ss-UBC-Opti-hRS1-1 |
| GOI-C28 | ss-CMV-Opti-hRS1-1 |
| GOI-C25 | ss-CMV-Opti-hRS1-1 |
| GOI-C45 | ss-CMV-Opti-hRS1-2 |
| GOI-C46 | ss-CMV-Opti-hRS1-3 |
| GOI-E11-V1 | ss-CMV-hRS1-WT |
| GOI-C59-V1 | ss-CAG(N-Intron)-Opti-hRS1-1 |
| GOI-C59-V2 | ss-CAG-Opti-hRS1-3 |
| GOI-C59-V3 | ss-CAG-WT-hRS1 |
| GOI-C21 | sc-CAG-Opti-hRS1-1 |

Four GOI plasmids (GOI-C26, GOI-C24, GOI-C59, GOI-C03) were transfected with 500 ng plasmid in 6-well plates. After 72 h, the cells and supernatants were collected respectively. The expression of RS1 in the above four different expression cassettes was detected by Western blot, as shown in FIGS. 2B-2D: the highest expression in supernatants and cells was GOI-C26 (CMV-hRS1 cassette), the amount of protein transiently expressed by the other three cassettes did not show a significant difference. To further verify the efficiency of in vivo expression of interested proteins through recombinant AAV packaged by different GOIs, a standard triple-plasmid AAV packaging protocol (see Example 2) was followed for preparation, wherein the corresponding capsid plasmid was RC-C08; as shown in FIGS. 1E-1G, at MOI of 200, RC-C08& GOI-C59 (ss-CAG-opti-hRS1) and RC-C08& GOI-C26 (ss-CMV-opti-hRS1) exhibited a higher expression of RS1 protein in the 293T supernatant and cells than that by the other two recombinant viruses. In the published data of clinical trials, the CMV promoter element is at carcinogenic risk. Therefore, the CAG element is preferred for the broad expression cassette in the development of ophthalmic gene therapy products.

### Example 2: Comparison of Transcription and Translation Efficiencies of Humanized Codon-Optimized RS1

The known construction scheme and process of the RC-C08 plasmid were as follows:

The sequence of capsid protein VP1 of RC-C08 serotype is as set forth in SEQ ID NO: 1, the sequences of the corresponding cap gene containing 7 mutation sites Q464V, A467P, D469N, 1470M, R471A, D472V, and S474G were as set forth in SEQ ID NO: 2. In addition, the capsid protein of RC-C08V2 serotype (VP1) is a mutant with Y500F added to VP1 of RC-C08.

In order to obtain the above designed virus, Shanghai Langjing Biotechnology Co., Ltd. was entrusted to carry out gene synthesis on Cap gene of RC-C08 serotype and RC-C08V2 serotype, with upstream and downstream cloning sequences simultaneously added, and was cloned into a pUC57 vector by T-A clone as a subsequent amplification template. The resulting vectors were designated as pUC—RC-C08-VP1 and pUC—RC-C08V2-VP1, respectively.

### 2.1 Amplication of Gene of Interest Fragment

The PCR reaction system as shown in Table 3 below was used to amplify the gene of interest fragment for the subsequent cloning step:

TABLE 3
PCR reaction system

| | 50 μl | | | |
|---|---|---|---|---|
| | Reaction | | | |
| Components | system | | Final concentration | |
| 5 × PrimeSTAR Buffer | 10 | μL | 1X | |
| (Mg2+ Plus) | | | | |
| 10 μM forward primer | 2.5 | μL | 0.5 | μM |
| 10 μM reverse primer | 2.5 | μL | 0.5 | μM |
| dNTP Mixture (each 2.5 mM) | 4 | μL | 200 | μM |
| Template DNA | 10 | ng | cDNA <1 μg; plasmid <10 ng | |
| PrimeSTAR HS DNA | 0.5 | μL | 0.02 | U/μL |
| Polymerase (Takara, 2.5 U/μl) | | | | |

| | |
|---|---|
| ddH2O | to 50 μL |

Wherein the forward primer sequence is: 5′-gacgtcagacgeggaagcttcgatc-3′ (SEQ ID NO: 49), the reverse primer sequence is: 5′-gctgtttaaacgcccgggctgtag-3′ (SEQ ID NO: 50). PCR Amplification procedure is shown in Table 4:

TABLE 4
PCR Amplification procedure

| | | |
|---|---|---|
| Steps | Temperature | Time |

| | | | | |
|---|---|---|---|---|
| Denaturation | 98° | C. | 180 | seconds |
| 34 cycles | 98° | C. | 10 | seconds |
| | 62° | C. | 15 | seconds |
| | 72° | C. | 160 | seconds |
| Final extension | 72° | C. | 5 | minutes |

| | | | |
|---|---|---|---|
| Hold | 4° | C. | Hold |

1) The PCR product was confirmed to be correct by electrophoresis using 1% Agarose gel, using the corresponding DNA marker as a control. A Gel band at the expected position of about 2.2K bp was cut for recovering the remaining PCR product using QIAquick Gel Extraction Kit (QIAGEN) according to the instructions, and the product concentration was determined to 86 ng/μl using Nano-300.
2) pRC2 vector (whole gene synthesized by Genescript) was subjected to Double Digestion (at 37° C. for 2 hours) in the following reaction system as shown in Table 5:

TABLE 5
Reaction System for digestion of pRC2 vector

| Components | 50 μL Reaction system | |
|---|---|---|
| 10 × CutSmart buffer | 5 | μL |
| pRC2 vector | 2 μL(2 μg) | |
| HindIII-HF | 1 | μL |
| XmaI | 1 | μL |
| H2O | 41 | μL |

3) The enzyme-digested product was confirmed to be correct by electrophoresis using 1% Agarose gel, using the corresponding DNA marker as a control. A Gel band at the expected position of about 2.2K bp was cut for recovering the remaining PCR product according to the instructions of QIAquick Gel Extraction Kit (QIAGEN), and the product concentration was determined to 20 ng/μl using Nano-300.
4) The above purified products were ligated (at 50° C. for 1 h) according to the reaction system shown in Table 6 below:

TABLE 6
Ligation system

10 μL Reaction system

| | Ligation | |
|---|---|---|
| Components | Reaction system | Control |
| MML assembly mix | 5 μL | 5 μL |
| pRC2 (Plasmid linearization) | 2 μL | 2 μL |
| Enzyme-digested RC-C08 VP1 | 1 μL | — |
| PCR products | | |
| ddH2O | 2 μL | 3 μL |

5) The ligation product was transformed into Stb13 Competent cells, with reference to Stb13 Chemically Competent Cell (Shanghai Weidi Biotechnology Co., Ltd.) instructions.
6) A single clone on LB (Kana) plate was picked to a sterile 1.5 mL tube added with 200 μl LB (Kana) culture medium in advance, followed by incubating at 37° C. at 250 rpm for 3 h, and then Colony PCR screening for positive clones according to the reaction system shown in Table 7 below:

TABLE 7
Screening system

| | 20 μL Reaction system | | | |
|---|---|---|---|---|
| Components | PCR screening | | Mock | |
| Premix Taq TaKaRa | 10 | μL | 10 | μL |
| Taq ™Version 2.0 plus dye (TaKaRa) | | | | |
| C08-WF primer (10 μM) | 1 | μL | 1 | μL |
| M13F universal primer (10 μM) | 1 | μL | 1 | μL |
| Bacteria solution | 2 | μL | — | |
| ddH2O | 6 | μL | 8 | μL |

Colony PCR procedure was shown in Table 8:

TABLE 8
Colony PCR procedure

| | | |
|---|---|---|
| Steps | Temperature | Time |

| | | | | |
|---|---|---|---|---|
| Initial | 94° | C. | 3 | min |
| Denaturation | | | | |
| 34 cysles | 94° | C. | 30 | seconds |
| | 55° | C. | 30 | seconds |
| | 72° | C. | 45 | seconds |
| Final extension | 72° | C. | 5 | minutes |

| | | | |
|---|---|---|---|
| Hold | 4° | C. | Hold |

7) Agarose gel electrophoresis identification.

Three recombinant clones (5 single colonies per clone) were confirmed by PCR with bacterial suspension, and all colonies were positive.
8) The positive clones (#1 #2 #3) were selected and sequenced.
9) After the sequencing results were aligned correctly, Plasmid was extracted using the TIANGEN EndoFree Maxi Plasmid Kit (TIANGEN DP117) according to the instructions.
   2.2 rAAV Viurs Adherent Packaging Step:

The GOI (gene of interest) plasmid used in the packaging process was transcribed and translated into RS1 protein (224aa), and the expression cassette of the GOI plasmid comprises an enhancer, a promoter, an intron, a polyadenylation region, and also comprises an ITR (L-ITR and R-ITR) sequence on each side of the expression cassette, thereby further providing the genome of the rAAV vector.

As shown in FIG. 2, human-derived wild-type RS1 and RS1 codon sequence optimized in different ways were constructed into the rAAV vector regulated by corresponding promoter and intron (FIG. 2A). Since the tissue distribution of the genetic material of rAAV in patients with rare diseases needs to be studied after clinical administration of XLRS, and most of the patients have point mutations in the RS1 gene, the human-derived RS1 gene can still be detected at the mRNA level. Therefore, it is necessary to perform codon optimization on the RS1 protein in product design and evaluate the protein translation efficiency after different codon optimizations. Subsequently, the recombinant AAV virus was packaged in HEK293T by co-transfection with triple-plasmid with combinations of the RC-C08 capsid and the above four different expression cassettes GOI (in FIG. 2-A). After density gradient purification with iodixanol, HEK293T cells were infected with the packaged AAV virus at a multiplicity of infection (MOI) of 2000vg/cell, and the supernatant and cell samples were harvested after 48 hours.

The triple-plasmid packaging AAV has the following operation processes:

Firstly, HEK293T cells were allowed to adhere to the culture dish after recovery, and when the confluency of HEK293T cells in the culture dish reached 90%, the cell line was passaged at a ratio of 1:3, followed by culturing for 24 h until the cell confluence reaches 80%, and carrying out plasmid transfection; suitable transfection systems were prepared before transfection, and for each 10 cm dish of the packaging system, the transfection mixture was preferably prepared according to the following system: serum-free Medium Opti-MEM (Gibco) 500 μl, HLP plasmid 15 μg (whole gene synthesized by Genescript), RC-C08 (or RC-C08V2) plasmid prepared as described above 7.5 μg, GOI plasmid 7.5 μg (prepared in Example 1), PEIpro (Polyplus) 22.5 μl; thirdly, transfection process: the transfection mixture was dropwise added into different areas of a 10 cm culture dish, followed by shaking up in a gentle cross manner; and fourthly, packaging and culturing: the transfected cells were transferred to a carbon dioxide incubator, and cultured for 72 h at 37° C.; and finally, collecting virus: 72 h after transfection, the cells were pipetted with cell supernatant and centrifuged at 1500 rpm to collect cell pellets; lysate was added for lysis in a shaker at 37° C. for 1 h, followed by centrifuging at 4000 rpm with a horizontal rotor for 10 min, the supernatant was collected, filtered with 0.45 μm needle filter, and subpackaged in 10 μl volume, and virus gene copy number (unit: vg) in cells was detected by real-time quantitative PCR method. The cell samples were lysed and subjected to non-denaturing WB test together with the supernatant (FIG. 2-B), and the results showed that the expression level of the RS1 polymer secreted protein is superior to that of other viruses after the HEK293T cells were infected with the GOI-C59 virus, and the expression level of the intracellular RS1 is weaker than that of other viruses. The Western Blot results were subjected to gray-scale scanning with Tubulin as internal reference and the relative quantitative value of RS1/Tubulin as the relative expression level of RS1 protein. The final results were consistent with the Western Blot results (FIGS. 2-C-D).

### LIST OF ABBREVIATIONS

| Abbreviation | |
|---|---|
| in English | Full name in English |
| CDS | Coding DNA sequence |
| scAAV | Self-complementary adeno-associated virus |
| ssAAV | Single-stranded adeno-associated virus |
| GOI | Gene of interest |
| RC | Rep and cap |
| ssRC-C08 | Single-stranded RC-C08 serotype |
| ssAAV8 | Single-stranded AAV8 serotype |
| ss7m8 | Single-stranded AAV2.7m8 serotype |
| MOI | Multiplicities of infection |
| IVT | Intravitreal injection |
| SR | Subretinal injection |
| RIPA | Radioimmunoprecipitation assay buffer |
| PAGE | Polyacrylamide gel electrophoresis |
| WB | Western Blot |
| SDS | Sodium dodecyl sulfate |
| XLRS | X-linked retinoschisis |
| qRT-PCR | Quantitational real-time polymerase chain reaction |
| RT-PCR | Reverse transcription polymerase chain reaction |
| PBS | Phosphate buffered saline-MgCl&KCl |
| DMEM | Dulbecco's modified eagle medium |
| FBS | Fetal bovine serum |
| PS | Penicillin-Streptomycin |

As shown in FIG. 2E: the expression cassette of GOI-C59 was further optimized: 1.GOI-C59V1: replacing the Chimeric Intron in the GOI-C59 plasmid expression cassette with EF In Intron; 2. GOI-C59V2: replacing opti-hRS1-1 in the GOI-C59 plasmid expression cassette with opti-hRS1-3; 3. GOI-C59V3: replacing opti-hRS1-1 in the GOI-C59 plasmid expression cassette with hRS1-WT; 4. GOI-C59V4: replacing the Chimeric intron in the expression cassette of the GOI-C59 plasmid with the LTR-EFIn intron (abbreviated as LTR-nIntron: SEQ ID NO: 25); 5. GOI-C59V5: replacing opti-hRS1-1 in the GOI-C59V4 plasmid expression cassette with opti-hRS1-3; infecting ARPE19 and 661W (purchased from YaJi Bological) with viruses corresponding to GOI-C59, GOI-C59V1, GOI-C59V2, GOI-C59V3, GOI-C59V4, and GOI-C59V5, respectively, collecting supernatant after 72 h, and detecting the strength of RS1 in vitro expression of different plasmids by WB. As shown in FIGS. 2F-2I, the results show that the change of the expression cassette elements can affect the expression strength of protein, and Rs1 polymer of GOI-C59V5 has the highest expression level, indicating that 5-LTR modification elements facilitates the transcription and translation process of the protein, and indicating that LTR-nIntron significantly increased protein expression by affecting the stability of RS1 mRNA, especially in retinal cells.

As shown in FIGS. 2G and 2I, when the same GOI (GOI-C59) and different capsids were subjected to a rAAV retinal cell in-vitro infection activity test, it was found from the expression of RS1 polymer that the in vitro transduction activity of RC-C08V2&GOI-C59 was stronger than that of RC-C08&GOI-C59, indicating that the Y500F mutation occurred in the VP1 of the RC-C08 capsid, significantly improved the viral transduction activity.

### Example 3: Efficiency of In Vitro Secretory Expression of hRS1 Multimeric Proteins by Different Serotypes

The virus was packaged in HEK293T cells by triple-plasmid co-transfection with ssAAV (GOI-C59) and its double-stranded complementary form, scAAV (GOI-C21), together with AAV8, RC-C08 capsid (FIG. 3A) respectively as well as HLP (see Example 2 for specific steps). After density gradient purification with iodixanol, HEK293T cells were infected with the packaged virus at MOI=200, MOI=2000, respectively, and the supernatant and cell were harvested after 48 hours. Similarly, cells were lysed and subjected to non-denaturing WB to detect RS1 octamer expression together with the supernatant (FIG. 3B). There is no significant difference in RS1 octamer between ssAAV8 and scAAV8 in supernatant and cells (FIG. 3D); the RC-C08 capsid has a stronger transduction efficiency for HEK293T cells than AAV8 capsid, that is, the RS1 octamer expression level of GOI-C59 encapsidated by RC-C08 capsid in the supernatant and cells is significantly higher than that of GOI-C59 encapsidated by AAV8 capsid (FIG. 3C).

### Example 4: Differences in Interested Protein Transcription Levels and Protein Expression Between ssAAV and scAAV (RC-C08&GOI-C59) in Cells

GOI-C59 (ssAAV) and GOI-C21 (scAAV) were packaged with RC-C08 capsid for virus preparation. After density gradient purification with iodixanol, HEK293T cells were infected with the packaged virus at MOI of 200vg/cell, and supernatants and cell samples were collected at 6, 12, 24, 48 and 72 h, respectively.

The relative transcription level of RS1 mRNA in cells was detected and calculated by qPCR technique on cell samples. GOI-C21 (scAAV) infected cells at low MOI and reached the peak of self transcriptional levels within 24 h (FIG. 4B), while GOI-C59 (ssAAV) required infection at higher MOI (MOI>100) for 48 h or longer to reach the peak of transcriptional levels (FIG. 4A). As the MOI of ssAAV increased, RS1 transcription levels increased significantly, while scAAV production increased only marginally. At the same time, scAAV transcription peaked at 24 h and started to decrease significantly after 48 h. However, at the same low multiplicity of infection (MOI=250), cells were respectively harvested at 6, 12, 24, 36, 48, 72 h after viral infection and subjected to qPCR assay. It was found that the intensity of hRS1 mRNA transcriptional levels of scAAV was higher than that of ssAAV at different time points (FIG. 4C). All cell samples and supernatant were subjected to non-denaturing WB to detect RS1 octamer expression (FIGS. 4D, F), and to grayscale scanning statistics to obtain the curve of RS1 octamer in cells and supernatants over time (FIGS. 4E, G). In the period of 6-24 h, the expression level of RS1 octamer of the cells infected by the ssAAV was slightly higher than that by the scAAV; in the period of 24-72 h, the expression level of RS1 octamer of the cells infected by the scAAV virus was obviously higher than that by the ssAAV, and the expression level of RS1 octamer of the cells infected by the scAAV and the scAAV basically has a positive correlation trend with time (FIGS. 4E, G).

As shown in FIG. 4, the statistical results showed that scAAV secreted more active hRS1 octamer in the late phase of infection in 293 cells as compared with ssAAV.

### Example 5: MRNA Transcription Level and Interested Protein Expression Capacity of ssRC-C08 and ssAAV2.7m8 in Photoreceptor Cells

RC-C08 capsid and AAV2.7m8 capsid (CN103561774B, the capsid protein sequence of AAV2.7m8 as set forth in SEQ ID NO: 19) with GOI-C59 and pHLP (AAV packaging helper plasmid from Genescript (Nanjing) whole gene synthesis) triple-plasmid were subjected to virus packaging, respectively, and purified by iodixanol density gradient centrifugation (as shown in Example 2). Two packaged viruses, RC-C08 and AAV2.7m8, were used to infect 661w cells at MOI=100, 1000, and 10000, respectively. The supernatant and cell samples were harvested at 48 h and 72 h. Total RNA was extracted from cells using TRIZOL. After reverse transcription into cDNA, the number of hRS1 mRNA copies was detected by RT-PCR and qPCR, thereby detecting the mRNA level of RS1, as shown in FIGS. 5A-5D.

FIG. 5 shows the intensity profile of hRS1 mRNA transcriptional levels over time after infection of 661W by AAV2.7m8 and RC-C08 virions at different MOIs.

The results show that MOI≥10³ led to an obvious expression of hRS1 mRNA, with the increase of virus dose, there was a significant increase in hRS1 mRNA expression level.

A Western Blot method was further applied, comprising the following specifical steps:
1. 48 hours after recombinant AAV virus infection, cells and supernatant were harvested, respectively, 120 μL of strong RIPA lysate (added with protease inhibitor) was added to the cells, and RIPA lysate was also added to the supernatant.
2. Sample preparation: the total volume of loading buffer, ddH2O and sample was 50 μL, with 33 μL of sample added. For denaturing gel, NuPAGE™ LDS sample buffer (4×), NuPAGE™ sample reducing agent (10×), and non-denaturing gel buffer were used for preparation in house.
3. Boiling samples: for denaturing gel the sample was boiled for 15 min at 98° C., and for non-denaturing gel the sample was not boiled.
4. Loading: the gel was loaded into the electrophoresis clip (ensuring that the concave line and the short plate must be in close contact), a running buffer was filled between the clips, and added in the electrophoresis chamber to higher than the bottom standard line. 15 μL of the protein sample and 5 μL of marker was added to the lane in order.
5. Electrophoresis: the initial voltage for electrophoresis was 80V, and the voltage was adjusted to 120V after the sample enters the separation gel. The electrophoresis time was controlled according to the molecular position of the pre-stained protein marker.
6. Transferring to the membrane: after the protein subjecting to SDS-PAGE electrophoresis, the PVDF membrane was activated with methanol and placed on an electroblotting device (Genescript) in the following order: sponge plate-PVDF membrane-gel-sponge plate (be careful to remove air bubbles), followed by putting into an electrotransfer tank, and transferring for 16 min for non-denaturing gel and a denaturing gel for 5 min.
7. Blocking and antibody incubation: after the electrotransfer was finished, the PVDF membrane was taken out and placed in a plate, about 10 ml of TBST blocking solution containing 5% of skimmed milk powder was added, followed by blocking for 60 min at room temperature. The blocking solution was discarded, RS1 antibody diluted 1:3000 as appropriate was added, followed by incubating overnight at 4° C. with shaking. The membrane was washed 3 times with TBST, 5 min each time.
8. Secondary antibody incubation: 1:5000 diluted goat anti-rabbit secondary antibody was added, followed by incubating for 1 h at normal temperature, the membrane was washed for 3 times with TBST, 10 min each time.

Color development: the solution A and the solution B in the ECL chromogenic substrate were uniformly mixed in a ratio of 1:1, a proper amount of uniformly mixed developing solution was covered on a PVDF membrane, followed by exposure imaging in a Biorad full-automatic chemiluminescence imaging system.

As shown in FIGS. 5E-5F, 661w cells infected with recombinant virus was detected by a WB test (denaturing and non-denaturing gel), it was found that significant RS1 expression was seen at both 48 h and 72 h after infection, and at 72 h, hRS1 protein expression after RC-C08 infection was significantly better than that after AAV2.7m8 infection with the same viral MOI. In the photoreceptor cell 661w, the expression of hRS1 protein mediated by RC-C08 serotype was significantly stronger than that mediated by AAV2.7m8 serotype.

### Example 6: Comparison of Expression Intensities of Interested Proteins in Retinal Choroid and Vitreous Humor in KO Mouse Model with ssRC-C08 and ssAAV8

Preparation Steps of ssRC-C08 and ssAAV8 with 293F Suspension Cell:

After 293F suspension cells were cultured for 2-4 days, samples were taken to count the density and viability of the cells. When the cell density was more than 1×10⁶ cells/ml and the cell viability was more than 90%, the cell was subjected to dilution passage treatment, and the treated cells were diluted by using fresh Dynamis™ Medium (Gibco) preheated in a water bath kettle at 37° C., maintaining the cell density at 0.N (N=1-5)×10⁶ cells/ml. The cells were cultured in a constant temperature shaker at 37° C., 5% CO₂ and 120 rpm; the cells used for transfection should be at least 4 generations after recovery and in the logarithmic growth phase, with a viability of more than 90% and a density of approximately 2×10⁶ cells/mL. On the day of transfection, samples were taken to count the cell density and viability. Preparation of transfection solution (30 mL system) with fresh Dynamis™ Medium pre-heated in a 37° C. water bath kettle: (1) 60 μg of plasmid (1×10⁶ cells/μg) was added to 1.5 mL of Dynamis™ Medium in proportion (helper plasmid (HLP plasmid): capsid plasmid (RC-C08 or AAV8): gene of interest plasmid (GOI plasmid)=2:1:0.3); (2) 60 μL of Fecto VIR-AAV was added to the plasmid dilution, mixed by shaking with vortexer for 3 seconds immediately, and left to stand at room temperature for 30 min. The transfection solution was added dropwise to the cell culture solution containing 300 μl PS (final concentration: 1%) and 30 μl 10% P188 (final concentration: 0.01%), and the flask was returned to the shaker at 37° C. and 5% CO₂ at 120 rpm for further culture. The cells were lysed after transfection 72 h, keeping the processing mode of the cell samples consistent with that of adherent cells, and the copy number of the viral genes (vg) was detected in the cells by real-time quantitative PCR. The virus ssRC-C08&GOI-C59 and ssAAV8&GOI-C59 were obtained by ultracentrifugation and purification.

### The Administration Method for Mouse by Intravitreal Injection:

The viruses ssRC-C08&GOI-C59 and ssAAV8&GOI-C59 obtained by the ultracentrifugation and purification were administrated intraocularly by IVT in C57 mouse, with the specific steps as follows:

Age-appropriate mice (3-4 mice per group, 6-8 weeks old, C57 balb/c (purchased from Shanghai Model Organisms Center, Inc.)) were intraperitoneally injected with 60 mg/kg of Zoletil®50, 7.98 mg/kg of Xylazine hydrochloride on the day of the experiment, which were prepared to the desired concentration with 0.9% sodium chloride injection. 0.5% of Tropicamide Phenylephrine Eye Drops (Mydrin-p) was used for mydriasis, and Ofloxacin Eye Ointment (Dikeluo) were smeared on the surface of eyes. Under an operation microscope, a disposable injection needle was used for puncturing the sclera on the inner side of the corneaoscleral edge, and a microsyringe with a 36G flat needle was used for entering along a puncture hole and bypassing the crystalline lens to reach the vitreous body to delivery the medicine by intravitreal injection. The mouse was placed in a warm environment for resuscitation after the injection, and the mouse was placed back into a cage box after the mouse was awakened. For each experimental group, injections were completed sequentially, and the time to start anesthesia and administration (the time when the injection was finished) were recorded. Unless otherwise specified, the left eye (OS) is a non-administered eye, and the right eye (OD) is an administered eye. During the intravitreal injection, any abnormality caused by manual operation, such as injection failure, intraocular injury, etc., will be faithfully recorded, and those with severe damage will be excluded.

Vitreous humor was extracted at day 14, day 21 and day 28 after IVT administration, followed by retinal choroidal tissue isolation, and the above samples were pretreated before protein loading and then subjected to WB test, where the right eye (OD) of the mouse was the administered eye and the left eye (OS) was the non-administered eye as a control. The results are shown in FIGS. 6A-D (B35× representing numbering of mice), which shows that RS1 protein was strongly expressed in the retina, choroid and vitreous humor of wild-type mice. There was no expression of RS1 protein in RS1 KO mouse retina & choroid and vitreous humor. Compared with the left eye, the right eye of the RS1 KO mouse injected with the ssRC-C08 and the ssAAV8 has obvious expression of RS1 protein in retina, choroid and vitreous humor, and the expression level of the interested protein after IVT injection of ssRC-C08 was significantly higher than that of the RS1 protein after IVT injection of ssAAV8, indicating that the transduction efficiency of RC-C08 in vivo was shown to be stronger than that of AAV8.

As shown in FIGS. 6A-6B, in the non-denaturing gel mouse eye tissue WB, it can be seen that multimeric RS1 protein was expressed in the virus-injected OD eyes, with multimeric RS1 being significantly more strongly expressed in group B (RC-C08) than in group A (AAV 8) at D28. The results of WB test in vitreous humor showed that on Day 14 and Day 21 after IVT administration, the multimeric RS1 protein secreted into the vitreous humor in the RC-C08 administration group was significantly higher than that in the AAV8 administration group; the difference in expression intensity of hRS1 in HEK293T cells at different multiplicity of infections of ssRC-C08/AAV8 (GOI-C59) in vitro (FIG. 6E).

The above results indicated that the RC-C08 serotype has a stronger ability to express RS1 polymer in vivo and in vitro than the AAV8 serotype (same GOI).

### Example 7: Comparison of RS1 Protein Expression Following IVT Administration of ssRC-C08 &GOI-C59 Drugs in Photoreceptor Cells and Model Mouse Eye Tissues

First, 661w cells were infected with the above purified virus ssRC-C08 (GOI-C59) at MOI=100, 1000, and 10000, and then supernatant and cell samples were harvested at 48 and 72 hours, respectively, and WB test was performed (see Example 5 for sample processing and detection methods), with the results shown in FIGS. 7A-D. WB results showed that ssRC-C08 rAAV infected photoreceptor cells in vitro, resulting in significant expression of hRS1 polymer protein (both cellular and secreted supernatant), and the secreted RS1 in 72 h supernatant was much higher than RS1 in cells. The expression of RS1 polymer protein showed a significant dose-dependent effect with increasing viral MOI. FIGS. 7A-7D show that: 1) the hRS1 polymers were significantly expressed in both cell and supernatant of the photoreceptor infected by the ssRC-C08; 2) the level of biologically active functional protein (hRS1 in octamer form) secreted into the supernatant was significantly higher than that in the cells; 3) as the MOI of RC-C08 virus increased, the expression of hRS1 polymer protein showed a significant dose-dependent effect in both cell samples and supernatants, and was positively correlated with the infection time. The right eyes (OD) of mice were dosed (ssRC-C08&GOI-C59) by two injections, intravitreal and subretinal, while the left eyes (OS) were not dosed as a control. Mouse retinochoroidal proteins were extracted separately for WB test (mouse selection, sample processing and detection methods referring to Example 6).

The method for the separation of mouse eyeball retina tissue comprises the following steps: the mice to be taken for tissue was anesthetized, the cervical vertebrae was dislocated, the eyeballs of the mice was extracted for stripping and a complex of retina, choroid and Retinal Pigment Epithelium (RPE) under a microscope, which was placed in an EP tube with the volume of 1.5 ml, frozen on dry ice, and stored in a −80° C. refrigerator until the time of secondary treatment of a sample.

As shown in FIGS. 7E-7H, RS1 protein was strongly expressed in both retina and choroid in wild-type mice, but has no expression in both retina and choroid in RS1 KO control mice. Specifically, as shown in FIGS. 7E-7F, it can be seen that the OD eyes injected with the virus in groups A, B, C and D all expressed high levels of RS1 octamer protein from non-denaturing gel mouse eyeball tissue WB. Grayscale scanning results showed that: octamer RS1 (in ocular tissues): the expression level of hRS1 in ssRC-C08 (GOI-C59) was lower in IVT administration than in subretinal administration, but the hRS1 expression in both administration methods was higher than WT mRS1. At the same dosage, the subretinal administration had better hRS1 protein expression than intravitreal administration. As shown in FIGS. 7G-7H, the results of WB immunoblotting showed that in the retinal tissue of the mice in the administration groups, the multimeric RS1 proteins were expressed in virus-injected OD eyes (high-dose E9vg/OD in group A and low-dose E8vg/OD in group B), wherein, the expression of hRS1 multimers in the high-dose group A was stronger than that in the low-dose dosed group, and the expression level in the high-dose group was close to that of normal mice. The results showed that after the ssRC-C08 medicament was administrated by adopting IVT, the expression of the multimeric hRS1 protein in mouse tissues had a dose-dependent effect with the virus injection amount.

The data of Example 7 show that the interested protein expression in the subretinal administration mode is significantly higher than that in the intravitreal administration mode, both of which show dose-dependent expression.

### Example 8: In Vivo Efficacy of ssRC-C08&GOI-C59 in RS1 Knockout Mouse Model

### Mouse Optical Coherence Tomography (OCT) Detection Method:

Rs1 KO mouse (see CN112899311A for preparation method) was injected intraperitoneally with 60 mg/kg of Zoletil®50, 7.98 mg/kg of Xylazine hydrochloride, which were prepared to the desired concentration with 0.9% sodium chloride injection. 0.5% of Tropicamide Phenylephrine Eye Drops (Mydrin-p) was used for mydriasis, and Ofloxacin Eye Ointment (Dikeluo) were smeared on the surface of eyes. Under an operation microscope, a disposable injection needle was used for puncturing the sclera on the inner side of the corneaoscleral edge, and a microsyringe with a 36G flat needle was used for entering along a puncture hole and bypassing the crystalline lens to reach the vitreous body or subretinal cavitation to delivery the medicine by intravitreal injection. As shown in Table 3, mice in group I were injected intravitreally with ssRC-C08&GOI-C59 at a dose of 2E8vg, mice in group II were injected with 4E7vg of ssRC-C08&GOI-C59 into the subretinal space of the right eye, and mice in group III were injected with the control virus (ssRC-C08&GOI-E04) by IVT.

The mice were subjected to routine anesthesia and Tropicamide Phenylephrine Eye Drops was administered to dilate the pupil, followed by making the mice lying prone on the test table, applying Ofloxacin Eye Ointment to protect the mouse cornea; the mouse in vivo imaging all-in-one machine was turned on, the optical fiber and imaging lens were connected, the desktop software was opened, followed by adjusting the mouse position and lens angle to take fundus photography and OCT images.

During the OCT test, any abnormity found in the mouse eyeball, such as the conditions including visible ocular surface damage, eyeball collapse, etc., will be recorded truthfully, and those with severe damage will be excluded.

TABLE 3
RS1 KO model murine dosing regimen

| | | Dosage | Titer | Volume | Administration | Animal | |
|---|---|---|---|---|---|---|---|
| Group | Drug | (vg/eye) | (vg/ml) | (μl/eye) | (right eye) | model | Number |
| I | ssRC-C08 | 2.0E8 | 1.0E11 | 2 | intravitreal | rs1 KO | 6 |
| | (GOI-C59) | | | | | | |
| II | ssRC-C08 | 4.0E7 | 2.0E10 | 2 | subretinal | rs1 KO | 6 |
| | (GOI-C59) | | | | | | |
| III | ssRC-C08 | 4.0E7 | 2.0E10 | 2 | intravitreal | rs1 KO | 6 |
| | (GOI-E04) | | | | | | |

From the OCT images of FIGS. 8A-8B (distal to optic disk) we can see that at day 28 post-injection, the luminal size of the eye dexter (OD) in group I (IVT, 2E8 vg/eye) and group II (subretinal, 4E7 vg/eye) was significantly smaller than that in the non-injected eye (OS). Before injection (FIG. 8A) and on the 28th day after injection (FIG. 8B), the size of the cavitation (OD) of the injected eye in group III KO mice was no different from that of the untreated eye (OS), and the cavitation area of the eye in the control group III had larger tear areas of left and right eyes.

Statistical results showed that comparing administration groups I and II with group III (AAV-EGFP), the OCT results of the distal and inferior ends of the optic disc showed that after 4 weeks, the size of the retinal cavitation in the injected eye (OD) in group I and group II (ssRC-C08&GOI-C59) was significantly smaller than that of the left eye control of the same mouse (FIGS. 8C-8D), which was close to the level of normal WT mice, especially group II (subretinal injection group) had the most significant efficacy, and the retinal tearing was fundamentally reversed in this short-term drug efficacy study.

### Example 9: Dose Effectiveness Assessment of ssRC-C08&GOI-C59 in Rs1 Knockout Model Mice (OCT&IF&HE)

ssRC-C08&GOI-C59 was injected into the eyes of Rs1 KO model mice at start dose 1.00E+7vg/eye steps (10-fold progressive increasing dose) as described in Example 6

On the 29th day after the subretinal injection, the mice were placed in a completely dark environment overnight (>8 hours), on the 30th day after the subretinal injection, 60 mg/kg of Zoletil®50 were injected intraperitoneally; the mice were anesthetized with 8 mg/kg of Xylazine hydrochloride, 0.5% of Tropicamide Phenylephrine Eye Drops (Mydrin-p) was used for mydriasis, and 0.4% of Oxybuprocaine Hydrochloride Eye Drops (Benoxil) were used for local anaesthesia of the eyes. After 5 minutes, the mice was completely anesthetized, and placed on an operation table, with a grounding electrode inserted into the tail, a reference electrode inserted into the left ear and the right ear, and an action electrode respectively placed in the left eye and the right eye, Scotopic 3.0 test was performed, Photopic 3.0 test was performed after being exposed for about 2 minutes under white light, and the ERG waveform of the mice was recorded and stored. During the ERG test, any abnormity found in the mouse eyeball, such as the conditions including visible ocular surface damage, eyeball collapse, etc., will be recorded truthfully, and those with severe damage will be excluded.

The mouse in vivo imaging all-in-one machine was turned on, the optical fiber and imaging lens were connected, the desktop software was opened; the mice were subjected to routine anesthesia and Tropicamide Phenylephrine Eye Drops was administered to dilate the pupil, followed by making the mice lying prone on the test table, applying Ofloxacin Eye Ointment to protect the mouse cornea, adjusting the mouse position and lens angle to take fundus photography and OCT images. During the OCT test, any abnormity found in the mouse eyeball, such as the conditions including visible ocular surface damage, eyeball collapse, etc., will be recorded truthfully, and those with severe damage will be excluded.

The immunofluorescence of the frozen section of the retina tissue from an experimental mouse is carried out by the following method:

The mice to be taken for tissue was anesthetized, the cervical vertebrae was dislocated, the eyeballs were extracted from the mice to be frozen and sliced, and placed in eyeball fixative for fixation overnight. The mouse eyeballs of mice were taken out carefully from the eyeball fixative with tweezers, and placed in a mold filled with an O.C.T. embedding agent (the placement should make the cornea face left), wherein the eyeball position was adjusted carefully by using a pipette tip. The mold containing the mouse eyeballs was placed in dry ice or a −80° C. refrigerator for about 30 minutes to allow the O.C.T. to completely solidify before demolding and slicing. The freezing microtome was adjusted to section mode, with a thickness to 12 μm. The cut slice was sticked on the slides by choosing the rolling collection method, that is, the first slice is stuck on the first slide, the second slice is stuck on the second slide, and so on. After 20 slides are stuck, the 21st slice was stuck on the first slide. The number of sections that can be carried by one slide (generally 3) can be roughly determined according to the size of the sample, and generally 5 sections with better morphology were selected for immunofluorescent staining.

As shown in FIG. 9A, the administration mode for I-V mice was as follows: RS1 ko model mice were administrated via intravitreal cavitation (IVT) injection about 3 weeks of birth, wherein each mouse was administrated in the right eye, with the left eye as a control; there was 6 mice per group, with an administration dosage of E10vg, E9vg, E8vg and E7vg from high to low, and fundus OCT examination was respectively carried out on W2, W4, and W6 after administration (FIG. 10A). As shown in FIG. 10C, the immunofluorescence markers of retina sections of the mice in each group indicated that RC-C08&GOI-C59 IVT injection can effectively transduce retina tissues and express the anaplerosis protein (RS1 protein marked by a wavelength of 594 nm), and the expression intensity of the protein is in positive correlation with the administration dosage;

As shown in FIG. 9B, RC-C08&GOI-C59 (IVT administration) significantly inhibited the formation of cavitation in all layers of the retina of mice (administration group I-III) in a dose-dependent manner, with an effective dose at 1E8VG/eye (FIGS. 9C & 9D); IVT injection of RC-C08 & GOI-C59 (as compared with subretinal administration) was also effective in alleviating the formation of inner nuclear layer cavitation and progression of the disease in a dose-dependent manner (FIGS. 9E & 9F); the formation of inner nuclear layer cavitation was smaller with the increase of dose in I-III groups, wherein there was no tear in all layers of the retina in group I, and the disease course was completely reversed; In the four groups (I-IV) of RC-C08 & GOI-C59 administered with IVT, the retinal local inflammatory response was weak in all three groups of mice except the E10vg group after 6 weeks of administration (FIG. 9G), indicating that it is safe and effective to use the IVT administration of RC-C08 & GOI-C59.

### Example 10: Long Term Efficacy Assessment of Drug Candidates in RS1 Knockout Mouse Model (OCT & ERG)

Long-term efficacy assessment of the drug candidates RC-C08& GOI-C59 were performed with reference to the OCT and ERG examination procedures as described in Examples 8 and 9. As can be seen from the OCT images (distal to optic disc) at 6 weeks after administration in FIGS. 10A-10B, on day 42 after injection, the tear of eyes dexter (OD) in group I (IVT, 2E8 vg/eye) and group II (subretinal, 4E7 vg/eye) did not show a significant decrease compared with control eyes, and had a certain correlation with the natural course of disease of Rs1 KO mice. However, by analyzing the data of the adminstered right eyes in groups II and III, it can be seen that the subretinal administration group still showed significant efficacy in cavitation tear relief or healing at the time point of 6 weeks after administration (W6). From the statistical results, it can be seen that the drug candidates can be administered either subretinally or by IVT, and the effective dose of IVT is about 10 times of that of subretinal.

The ERG examination results of the three administration groups (I-III) and WT mice were further analyzed, as shown in FIGS. 10C-10D: compared with the group III (AAV-EGFP), the ERG visual function of the administration group I and the administration group II has recovered to a certain degree, the efficacy of the SR administration group was stronger than that of the IVT administration group, tending to the b/a value of WT normal mice. The visual function of mice has been improved to a certain extent.

The specific sequence information is as follows:

| SEQ | | |
|---|---|---|
| ID | Sequence | |
| NO | Description | Specific sequence |
| 1 | Capsid | MAADGYLPDWLEDTLSEGIRQWWKLKPGPPPPKPAERHKDDSRGL |
| | protein VP1 | VLPGYKYLGPFNGLDKGEPVNEADAAALEHDKAYDRQLDSGDNPY |
| | protein of | LKYNHADAEFQERLKEDTSFGGNLGRAVFQAKKRVLEPLGLVEEP |
| | RC-C08 | VKTAPGKKRPVEHSPVEPDSSSGTGKAGQQPARKRLNFGQTGDAD |
| | serotype | SVPDPQPLGQPPAAPSGLGTNTMATGSGAPMADNNEGADGVGNSS |
| | | GNWHCDSTWMGDRVITTSTRTWALPTYNNHLYKQISSQSGASNDN |
| | | HYFGYSTPWGYFDF |
| | | NRFHCHFSPRDWQRLINNNWGFRPKRLNFKLFNIQVKEVTQNDGT |
| | | TTIANNLTSTVQVFTDSEYQLPYVLGSAHQGCLPPFPADVFMVPQ |
| | | YGYLTLNNGSQAVGRSSFYCLEYFPSQMLRTGNNFTFSYTFEDVP |
| | | FHSSYAHSQSLDRLMNPLIDQYLYYLSRTNTPSGTTTQSRLQFSV |
| | | AGPSNMAVQGRNWLPGPCYRQQRVSKTSADNNNSEYAWTGATKYH |
| | | LNGRDSLVNPGPAMASHKDDEEKFFPQSGVLIFGKQGSEKTNVDI |
| | | EKVMITDEEEIRTTNPVATEQYGSVSTNLQRGNRQAATADVNTQG |
| | | VLPGMVWQDRDVYLQGPIWAKIPHTDGHFHPSPLMGGFGLKHPPP |
| | | QILIKNTPVPANPSTTFSAAKFASFITQYSTGQVSVEIEWELQKE |
| | | NSKRWNPEIQYTSNYNKSVNVDFTVDINGVYSEPRPIGTRYLTRN |
| | | L* |
| 2 | Capsid | atggctgccgatggttatcttccagattggctcgaggacactctc |
| | protein VP1 | tctgaaggaataagacagtggtggaagctcaaacctggcccacca |
| | gene of | ccaccaaagcccgcagagcggcataaggacgacagcaggggtctt |
| | RC-C08 | gtgcttcctgggtacaagtacctcggacccttcaacggactcgac |
| | serotype | aagggagagccggtcaacgaggcagacgccgcggccctcgagcac |
| | | gacaaagcctacgaccggcagctcgacagcggagacaacccgtac |
| | | ctcaagtacaaccacgccgacgcggagtttcaggagcgccttaaa |
| | | gaagatacgtcttttgggggcaacctcggacgagcagtcttccag |
| | | gcgaaaaagagggttcttgaacctctgggcctggttgaggaacct |
| | | gttaagacggctccgggaaaaaagaggccggtagagcactctcct |
| | | gtggagccagactcctcctcgggaaccggaaaggcgggccagcag |
| | | cctgcaagaaaaagattgaattttggtcagactggagacgcagac |
| | | tcagtacctgacccccagcctctcggacagccaccagcagccccc |
| | | tctggtctgggaactaatacgatggctacaggcagtggcgcacca |
| | | atggcagacaataacgagggcgccgacggagtgggtaattcctcg |
| | | ggaaattggcattgcgattccacatggatgggcgacagagtcatc |
| | | accaccagcacccgaacctgggccctgcccacctacaacaaccac |
| | | ctctacaaacaaatttccagccaatcaggagcctcgaacgacaat |
| | | cactactttggctacagcaccccttgggggtattttgacttcaac |
| | | agattccactgccacttttcaccacgtgactggcaaagactcatc |
| | | aacaacaactggggattccgacccaagagactcaacttcaagctc |
| | | tttaacattcaagtcaaagaggtcacgcagaatgacggtacgacg |
| | | acgattgccaataaccttaccagcacggttcaggtgtttactgac |
| | | tcggagtaccagctcccgtacgtccteggctcggcgcatcaagga |
| | | tgcctcccgccgttcccagcagacgtcttcatggtgccacagtat |
| | | ggatacctcaccctgaacaacgggagtcaggcagtaggacgctct |
| | | tcattttactgcctggagtactttccttctcagatgctgcgtacc |
| | | ggaaacaactttaccttcagctacacttttgaggacgttcctttc |
| | | cacagcagctacgctcacagccagagtctggaccgtctcatgaat |
| | | cctctcatcgaccagtacctgtattacttgagcagaacaaacact |
| | | ccaagtggaaccaccacgcagtcaaggcttcagttttctGTGgcc |
| | | ggaCCGagtAACATGGCGGTCcagGGTaggaactggcttcctgga |
| | | ccctgttaccgccagcagcgagtatcaaagacatctgcggataac |
| | | aacaacagtgaaTACGCGtggactggagctaccaagtaccacctc |
| | | aatggcagagactctctggtgaatccgggcccggccatggcaagc |
| | | cacaaggacgatgaagaaaagttttttcctcagagcggggttctc |
| | | atctttgggaagcaaggctcagagaaaacaaatgtggacattgaa |
| | | aaggtcatgattacagacgaagaggaaatcaggacaaccaatccc |
| | | gtggctacggagcagtatggttctgtatctaccaacctccagaga |
| | | ggcaacagacaagcagctaccgcagatgtcaacacacaaggcgtt |
| | | cttccaggcatggtctggcaggacagagatgtgtaccttcagggg |
| | | cccatctgggcaaagattccacacacggacggacattttcacccc |
| | | tctcccctcatgggtggattcggacttaaacaccctcctccacag |
| | | attctcatcaagaacaccccggtacctgcgaatccttcgaccacc |
| | | ttcagtgcggcaaagtttgcttccttcatcacacagtactccacg |
| | | ggacaggtcagcgtggagatcgagtgggagctgcagaaggaaaac |
| | | agcaaacgctggaatcccgaaattcagtacacttccaactacaac |
| | | aagtctgttaatgtggactttactgtggacactaatggcgtgtat |
| | | tcagagcctcgccccattggcaccagatacctgactcgtaatctg |
| | | taa |
| 3 | GOI-C59 | AGCGCCCAATACGCAAACCGCCTCTCCCCGCGCGTTGGCCGATTC |
| | Plasmid | ATTAATGCAGCTGGCACGACAGGTTTCCCGACTGGAAAGCGGGCA |
| | sequence | GTGAGCGCAACGCAATTAATGTGAGTTAGCTCACTCATTAGGCAC |
| | | CCCAGGCTTTACACTTTATGCTTCCGGCTCGTATGTTGTGTGGAA |
| | | TTGTGAGCGGATAACAATTTCACACAGGAAACAGCTATGACCATG |
| | | ATTACGAATTGCCTGCAGGCAGCTGCGCGCTCGCTCGCTCACTGA |
| | | GGCCGCCCGGGCAAAGCCCGGGCGTCGGGCGACCTTTGGTCGCCC |
| | | GGCCTCAGTGAGCGAGCGAGCGCGCAGAGAGGGAGTGGCCAACTC |
| | | CATCACTAGGGGTTCCTATCGATATCAAGCTTCGTTACATAACTT |
| | | ACGGTAAATGGCCCGCCTGGCTGACCGCCCAACGACCCCCGCCCA |
| | | TTGACGTCAATAATGACGTATGTTCCCATAGTAACGCCAATAGGG |
| | | ACTTTCCATTGACGTCAATGGGTGGACTATTTACGGTAAACTGCC |
| | | CACTTGGCAGTACATCAAGTGTATCATATGCCAAGTACGCCCCCT |
| | | ATTGACGTCAATGACGGTAAATGGCCCGCCTGGCATTATGCCCAG |
| | | TACATGACCTTATGGGACTTTCCTACTTGGCAGTACATCTACGTA |
| | | TTAGTCATCGCTATTACCATGGGTCGAGGTGAGCCCCACGTTCTG |
| | | CTTCACTCTCCCCATCTCCCCCCCCTCCCCACCCCCAATTTTGTA |
| | | TTTATTTATTTTTTAATTATTTTGTGCAGCGATGGGGGCGGGGGG |
| | | GGGGGGGGCGCGCGCCAGGCGGGGCGGGGCGGGGCGAGGGGCGGG |
| | | GCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGC |
| | | CCTATAAAAAGCGAAGCGCGCGGCGGGCGGATATCGGAGTCGCTG |
| | | CGTTGCCTTCGCCCCGTGCCCCGCTCCGCGCCGCCTCGCGCCGCC |
| | | CGCCCCGGCTCTGACTGACCGCGTTACTCCCACAGGTGAGCGGGC |
| | | GGGACGGCCCTTCTCCTCCGGGCTGTAATTAGCGCTTGGTTTAAT |
| | | GACGGCTCGTTTCTTTTCTGTGGCTGCGTGAAAGCCTTAAAGGGC |
| | | TCCGGGAGGGCCCTTTGTGCGGGGGGGAGCGGCTCGGGGGGTGCG |
| | | TGCGTGTGTGTGTGCGTGGGGAGCGCCGCGTGCGGCCCGCGCTGC |
| | | CCGGCGGCTGTGAGCGCTGCGGGCGCGGCGCGGGGCTTTGTGCGC |
| | | TCCGCGTGTGCGCGAGGGGAGCGCGGCCGGGGGCGGTGCCCCGCG |
| | | GTGCGGGGGGGCTGCGAGGGGAACAAAGGCTGCGTGCGGGGTGTG |
| | | TGCGTGGGGGGGTGAGCAGGGGGTGTGGGCGCGGCGGTCGGGCTG |
| | | TAACCCCCCCCTGCACCCCCCTCCCCGAGTTGCTGAGCACGGCCC |
| | | GGCTTCGGGTGCGGGGCTCCGTGCGGGGCGTGGCGCGGGGCTCGC |
| | | CGTGCCGGGCGGGGGGTGGCGGCAGGTGGGGGTGCCGGGCGGGGC |
| | | GGGGCCGCCTCGGGCCGGGGAGGGCTCGGGGGAGGGGCGCGGCGG |
| | | CCCCGGAGCGCCGGCGGCTGTCGAGGCGCGGCGAGCCGCAGCCAT |
| | | TGCCTTTTATGGTAATCGTGCGAGAGGGCGCAGGGACTTCCTTTG |
| | | TCCCAAATCTGGCGGAGCCGAAATCTGGGAGGCGCCGCCGCACCC |
| | | CCTCTAGCGGGCGCGGGCGAAGCGGTGCGGCGCCGGCAGGAAGGA |
| | | AATGGGCGGGGAGGGCCTTCGTGCGTCGCCGCGCCGCCGTCCCCT |
| | | TCTCCATCTCCAGCCTCGGGACTGCCGCAGGGGGACGGCTGCCTT |
| | | CGGGGGGGACGGGGCAGGGCGGGGTTCGGCTTCTGGCGTGTGACC |
| | | GGCGGCTCTAGAGCCTCTGCTAACCATGTTCATGCCTTCTTCTTT |
| | | TTCCTACAGACCGGTCTCCTGGGCAACGTGCTGGTTATTGTGCTG |
| | | TCTCATCATTTTGGCAAAGAATTCCACCGCCACCATGAGCCGGAA |
| | | GATCGAGGGCTTCCTGCTGCTGCTGCTGTTTGGATACGAGGCCAC |
| | | CCTGGGACTGTCCTCCACCGAGGACGAGGGCGAGGACCCCTGGTA |
| | | CCAGAAGGCCTGCAAGTGCGACTGCCAGGGCGGGCCTAACGCCCT |
| | | GTGGAGCGCTGGAGCTACCAGCCTGGACTGCATTCCCGAGTGCCC |
| | | ATATCACAAGCCCCTGGGATTCGAGAGCGGAGAGGTGACCCCAGA |
| | | CCAGATTACATGCAGTAACCCAGAACAGTATGTGGGATGGTATTC |
| | | AAGCTGGACCGCCAACAAGGCCAGACTGAACAGCCAGGGCTTCGG |
| | | CTGCGCCTGGCTGAGCAAGTTCCAGGACAGCAGCCAGTGGCTGCA |
| | | GATTGACCTGAAGGAGATCAAGGTGATCTCAGGGATCCTGACCCA |
| | | GGGAAGATGCGACATCGATGAGTGGATGACCAAGTACAGCGTGCA |
| | | GTACAGAACCGACGAAAGACTGAACTGGATCTACTACAAAGACCA |
| | | GACAGGAAACAACAGAGTGTTCTACGGCAACAGCGACAGAACCAG |
| | | CACCGTGCAGAACCTGCTGAGACCCCCCATCATCAGCAGATTCAT |
| | | CAGACTGATCCCCCTGGGGTGGCACGTGAGAATCGCAATCAGAAT |
| | | GGAGCTGCTGGAATGCGTGAGCAAGTGCGCCTGAGGATCCACGGG |
| | | TGGCATCCCTGTGACCCCTCCCCAGTGCCTCTCCTGGCCCTGGAA |
| | | GTTGCCACTCCAGTGCCCACCAGCCTTGTCCTAATAAAATTAAGT |
| | | TGCATCATTTTGTCTGACTAGGTGTCCTTCTATAATATTATGGGG |
| | | TGGAGGGGGGTGGTATGGAGCAAGGGGCAAGTTGGGAAGACAACC |
| | | TGTAGGGCCTGCGGGGTCTATTGGGAACCAAGCTGGAGTGCAGTG |
| | | GCACAATCTTGGCTCACTGCAATCTCCGCCTCCTGGGTTCAAGCG |
| | | ATTCTCCTGCCTCAGCCTCCCGAGTTGTTGGGATTCCAGGCATGC |
| | | ATGACCAGGCTCAGCTAATTTTTGTTTTTTTGGTAGAGACGGGGT |
| | | TTCACCATATTGGCCAGGCTGGTCTCCAACTCCTAATCTCAGGTG |
| | | ATCTACCCACCTTGGCCTCCCAAATTGCTGGGATTACAGGCGTGA |
| | | ACCACTGCTCCCTTCCCTGTCCTTATCGATAGATCTAGGAACCCC |
| | | TAGTGATGGAGTTGGCCACTCCCTCTCTGCGCGCTCGCTCGCTCA |
| | | CTGAGGCCGGGCGACCAAAGGTCGCCCGACGCCCGGGCTTTGCCC |
| | | GGGCGGCCTCAGTGAGCGAGCGAGCGCGCAGCTGCCTGCAGGCAG |
| | | CTTGGCACTGGCCGTCGTTTTACAACGTCGTGACTGGGAAAACCC |
| | | TGGCGTTACCCAACTTAATCGCCTTGCAGCACATCCCCCTTTCGC |
| | | CAGCTGGCGTAATAGCGAAGAGGCCCGCACCGATCGCCCTTCCCA |
| | | ACAGTTGCGCAGCCTGAATGGCGAATGGCGCCTGATGCGGTATTT |
| | | TCTCCTTACGCATCTGTGCGGTATTTCACACCGCATACGTCAAAG |
| | | CAACCATAGTACGCGCCCTGTAGCGGCGCATTAAGCGCGGCGGGT |
| | | GTGGTGGTTACGCGCAGCGTGACCGCTACACTTGCCAGCGCCCTA |
| | | GCGCCCGCTCCTTTCGCTTTCTTCCCTTCCTTTCTCGCCACGTTC |
| | | GCCGGCTTTCCCCGTCAAGCTCTAAATCGGGGGCTCCCTTTAGGG |
| | | TTCCGATTTAGTGCTTTACGGCACCTCGACCCCAAAAAACTTGAT |
| | | TTGGGTGATGGTTCACGTAGTGGGCCATCGCCCTGATAGACGGTT |
| | | TTTCGCCCTTTGACGTTGGAGTCCACGTTCTTTAATAGTGGACTC |
| | | TTGTTCCAAACTGGAACAACACTCAACCCTATCTCGGGCTATTCT |
| | | TTTGATTTATAAGGGATTTTGCCGATTTCGGCCTATTGGTTAAAA |
| | | AATGAGCTGATTTAACAAAAATTTAACGCGAATTTTAACAAAATA |
| | | TTAACGTTTACAATTTTATGGTGCACTCTCAGTACAATCTGCTCT |
| | | GATGCCGCATAGTTAAGCCAGCCCCGACACCCGCCAACACCCGCT |
| | | GACGCGCCCTGACGGGCTTGTCTGCTCCCGGCATCCGCTTACAGA |
| | | CAAGCTGTGACCGTCTCCGGGAGCTGCATGTGTCAGAGGTTTTCA |
| | | CCGTCATCACCGAAACGCGCGAGACGAAAGGGCCTCGTGATACGC |
| | | CTATTTTTATAGGTTAATGTCATGATAATAATGGTTTCTTAGACG |
| | | TCAGGTGGCACTTTTCGGGGAAATGTGCGGAACCCCTATTTGTTT |
| | | ATTTTTCTAAATACATTCAAATATGTATCCGCTCATGAATTAATT |
| | | CTTAGAAAAACTCATCGAGCATCAAATGAAACTGCAATTTATTCA |
| | | TATCAGGATTATCAATACCATATTTTTGAAAAAGCCGTTTCTGTA |
| | | ATGAAGGAGAAAACTCACCGAGGCAGTTCCATAGGATGGCAAGAT |
| | | CCTGGTATCGGTCTGCGATTCCGACTCGTCCAACATCAATACAAC |
| | | CTATTAATTTCCCCTCGTCAAAAATAAGGTTATCAAGTGAGAAAT |
| | | CACCATGAGTGACGACTGAATCCGGTGAGAATGGCAAAAGTTTAT |
| | | GCATTTCTTTCCAGACTTGTTCAACAGGCCAGCCATTACGCTCGT |
| | | CATCAAAATCACTCGCATCAACCAAACCGTTATTCATTCGTGATT |
| | | GCGCCTGAGCGAGACGAAATACGCGATCGCTGTTAAAAGGACAAT |
| | | TACAAACAGGAATCGAATGCAACCGGCGCAGGAACACTGCCAGCG |
| | | CATCAACAATATTTTCACCTGAATCAGGATATTCTTCTAATACCT |
| | | GGAATGCTGTTTTCCCGGGGATCGCAGTGGTGAGTAACCATGCAT |
| | | CATCAGGAGTACGGATAAAATGCTTGATGGTCGGAAGAGGCATAA |
| | | ATTCCGTCAGCCAGTTTAGTCTGACCATCTCATCTGTAACATCAT |
| | | TGGCAACGCTACCTTTGCCATGTTTCAGAAACAACTCTGGCGCAT |
| | | CGGGCTTCCCATACAATCGATAGATTGTCGCACCTGATTGCCCGA |
| | | CATTATCGCGAGCCCATTTATACCCATATAAATCAGCATCCATGT |
| | | TGGAATTTAATCGCGGCCTAGAGCAAGACGTTTCCCGTTGAATAT |
| | | GGCTCATAACACCCCTTGTATTACTGTTTATGTAAGCAGACAGTT |
| | | TTATTGTTCATGACCAAAATCCCTTAACGTGAGTTTTCGTTCCAC |
| | | TGAGCGTCAGACCCCGTAGAAAAGATCAAAGGATCTTCTTGAGAT |
| | | CCTTTTTTTCTGCGCGTAATCTGCTGCTTGCAAACAAAAAAACCA |
| | | CCGCTACCAGCGGTGGTTTGTTTGCCGGATCAAGAGCTACCAACT |
| | | CTTTTTCCGAAGGTAACTGGCTTCAGCAGAGCGCAGATACCAAAT |
| | | ACTGTTCTTCTAGTGTAGCCGTAGTTAGGCCACCACTTCAAGAAC |
| | | TCTGTAGCACCGCCTACATACCTCGCTCTGCTAATCCTGTTACCA |
| | | GTGGCTGCTGCCAGTGGCGATAAGTCGTGTCTTACCGGGTTGGAC |
| | | TCAAGACGATAGTTACCGGATAAGGCGCAGCGGTCGGGCTGAACG |
| | | GGGGGTTCGTGCACACAGCCCAGCTTGGAGCGAACGACCTACACC |
| | | GAACTGAGATACCTACAGCGTGAGCTATGAGAAAGCGCCACGCTT |
| | | CCCGAAGGGAGAAAGGCGGACAGGTATCCGGTAAGCGGCAGGGTC |
| | | GGAACAGGAGAGCGCACGAGGGAGCTTCCAGGGGGAAACGCCTGG |
| | | TATCTTTATAGTCCTGTCGGGTTTCGCCACCTCTGACTTGAGCGT |
| | | CGATTTTTGTGATGCTCGTCAGGGGGGCGGAGCCTATGGAAAAAC |
| | | GCCAGCAACGCGGCCTTTTTACGGTTCCTGGCCTTTTGCTGGCCT |
| | | TTTGCTCACATGTTCTTTCCTGCGTTATCCCCTGATTCTGTGGAT |
| | | AACCGTATTACCGCCTTTGAGTGAGCTGATACCGCTCGCCGCAGC |
| | | CGAACGACCGAGCGCAGCGAGTCAGTGAGCGAGGAAGCGGAAG |
| 4 | L-ITR | CCTGCAGGCAGCTGCGCGCTCGCTCGCTCACTGAGGCCGCCCGGG |
| | sequence(G | CAAAGCCCGGGCGTCGGGCGACCTTTGGTCGCCCGGCCTCAGTGA |
| | OI-C59 | GCGAGCGAGCGCGCAGAGAGGGAGTGGCCAACTCCATCACTAGGG |
| | Plasmid | GTTCCT |
| | expression | |
| | cassette) | |
| 5 | CMV | CGTTACATAACTTACGGTAAATGGCCCGCCTGGCTGACCGCCCAA |
| | Enhancer | CGACCCCCGCCCATTGACGTCAATAATGACGTATGTTCCCATAGT |
| | sequence(G | AACGCCAATAGGGACTTTCCATTGACGTCAATGGGTGGACTATTT |
| | OI-C59 or | ACGGTAAACTGCCCACTTGGCAGTACATCAAGTGTATCATATGCC |
| | GOI-C21 | AAGTACGCCCCCTATTGACGTCAATGACGGTAAATGGCCCGCCTG |
| | Plasmid | GCATTATGCCCAGTACATGACCTTATGGGACTTTCCTACTTGGCA |
| | expression | GTACATCTACGTATTAGTCATCGCTATTACCATG |
| | cassette) | |
| 6 | Chicken | TCGAGGTGAGCCCCACGTTCTGCTTCACTCTCCCCATCTCCCCCC |
| | beta actin | CCTCCCCACCCCCAATTTTGTATTTATTTATTTTTTAATTATTTT |
| | promoter(G | GTGCAGCGATGGGGGCGGGGGGGGGGGGGGCGCGCGCCAGGCGGG |
| | OI-C59 or | GCGGGGCGGGGCGAGGGGCGGGGCGGGGCGAGGCGGAGAGGTGCG |
| | GOI-C21 | GCGGCAGCCAATCAGAGCGGCGCGCTCCGAAAGTTTCCTTTTATG |
| | Plasmid | GCGAGGCGGCGGCGGCGGCGGCCCTATAAAAAGCGAAGCGCGCGG |
| | expression | CGGGCG |
| | cassette) | |
| 7 | chimeric | GGAGTCGCTGCGTTGCCTTCGCCCCGTGCCCCGCTCCGCGCCGCC |
| | intron | TCGCGCCGCCCGCCCCGGCTCTGACTGACCGCGTTACTCCCACAG |
| | (GOI-C59 | GTGAGCGGGCGGGACGGCCCTTCTCCTCCGGGCTGTAATTAGCGC |
| | Plasmid | TTGGTTTAATGACGGCTCGTTTCTTTTCTGTGGCTGCGTGAAAGC |
| | expression | CTTAAAGGGCTCCGGGAGGGCCCTTTGTGCGGGGGGGAGCGGCTC |
| | cassette) | GGGGGGTGCGTGCGTGTGTGTGTGCGTGGGGAGCGCCGCGTGCGG |
| | | CCCGCGCTGCCCGGCGGCTGTGAGCGCTGCGGGCGCGGCGCGGGG |
| | | CTTTGTGCGCTCCGCGTGTGCGCGAGGGGAGCGCGGCCGGGGGCG |
| | | GTGCCCCGCGGTGCGGGGGGGCTGCGAGGGGAACAAAGGCTGCGT |
| | | GCGGGGTGTGTGCGTGGGGGGGTGAGCAGGGGGTGTGGGCGCGGC |
| | | GGTCGGGCTGTAACCCCCCCCTGCACCCCCCTCCCCGAGTTGCTG |
| | | AGCACGGCCCGGCTTCGGGTGCGGGGCTCCGTGCGGGGCGTGGCG |
| | | CGGGGCTCGCCGTGCCGGGCGGGGGGTGGCGGCAGGTGGGGGTGC |
| | | CGGGCGGGGCGGGGCCGCCTCGGGCCGGGGAGGGCTCGGGGGAGG |
| | | GGCGCGGCGGCCCCGGAGCGCCGGCGGCTGTCGAGGCGCGGCGAG |
| | | CCGCAGCCATTGCCTTTTATGGTAATCGTGCGAGAGGGCGCAGGG |
| | | ACTTCCTTTGTCCCAAATCTGGCGGAGCCGAAATCTGGGAGGCGC |
| | | CGCCGCACCCCCTCTAGCGGGCGCGGGCGAAGCGGTGCGGCGCCG |
| | | GCAGGAAGGAAATGGGCGGGGAGGGCCTTCGTGCGTCGCCGCGCC |
| | | GCCGTCCCCTTCTCCATCTCCAGCCTCGGGACTGCCGCAGGGGGA |
| | | CGGCTGCCTTCGGGGGGGACGGGGCAGGGCGGGGTTCGGCTTCTG |
| | | GCGTGTGACCGGCGGCTCTAGAGCCTCTGCTAACCATGTTCATGC |
| | | CTTCTTCTTTTTCCTACAG |
| 8 | hGH | GGGTGGCATCCCTGTGACCCCTCCCCAGTGCCTCTCCTGGCCCTG |
| | poly(A) | GAAGTTGCCACTCCAGTGCCCACCAGCCTTGTCCTAATAAAATTA |
| | signal | AGTTGCATCATTTTGTCTGACTAGGTGTCCTTCTATAATATTATG |
| | polyadenylation | GGGTGGAGGGGGGTGGTATGGAGCAAGGGGCAAGTTGGGAAGACA |
| | region | ACCTGTAGGGCCTGCGGGGTCTATTGGGAACCAAGCTGGAGTGCA |
| | (GOI-C59 | GTGGCACAATCTTGGCTCACTGCAATCTCCGCCTCCTGGGTTCAA |
| | Plasmid | GCGATTCTCCTGCCTCAGCCTCCCGAGTTGTTGGGATTCCAGGCA |
| | expression | TGCATGACCAGGCTCAGCTAATTTTTGTTTTTTTGGTAGAGACGG |
| | cassette) | GGTTTCACCATATTGGCCAGGCTGGTCTCCAACTCCTAATCTCAG |
| | | GTGATCTACCCACCTTGGCCTCCCAAATTGCTGGGATTACAGGCG |
| | | TGAACCACTGCTCCCTTCCCTGTCCTT |
| 9 | Kanamycin | TTAGAAAAACTCATCGAGCATCAAATGAAACTGCAATTTATTCAT |
| | resistance | ATCAGGATTATCAATACCATATTTTTGAAAAAGCCGTTTCTGTAA |
| | marker | TGAAGGAGAAAACTCACCGAGGCAGTTCCATAGGATGGCAAGATC |
| | (GOI-C59 | CTGGTATCGGTCTGCGATTCCGACTCGTCCAACATCAATACAACC |
| | Plasmid | TATTAATTTCCCCTCGTCAAAAATAAGGTTATCAAGTGAGAAATC |
| | expression | ACCATGAGTGACGACTGAATCCGGTGAGAATGGCAAAAGTTTATG |
| | cassette) | CATTTCTTTCCAGACTTGTTCAACAGGCCAGCCATTACGCTCGTC |
| | | ATCAAAATCACTCGCATCAACCAAACCGTTATTCATTCGTGATTG |
| | | CGCCTGAGCGAGACGAAATACGCGATCGCTGTTAAAAGGACAATT |
| | | ACAAACAGGAATCGAATGCAACCGGCGCAGGAACACTGCCAGCGC |
| | | ATCAACAATATTTTCACCTGAATCAGGATATTCTTCTAATACCTG |
| | | GAATGCTGTTTTCCCGGGGATCGCAGTGGTGAGTAACCATGCATC |
| | | ATCAGGAGTACGGATAAAATGCTTGATGGTCGGAAGAGGCATAAA |
| | | TTCCGTCAGCCAGTTTAGTCTGACCATCTCATCTGTAACATCATT |
| | | GGCAACGCTACCTTTGCCATGTTTCAGAAACAACTCTGGCGCATC |
| | | GGGCTTCCCATACAATCGATAGATTGTCGCACCTGATTGCCCGAC |
| | | ATTATCGCGAGCCCATTTATACCCATATAAATCAGCATCCATGTT |
| | | GGAATTTAATCGCGGCCTAGAGCAAGACGTTTCCCGTTGAATATG |
| | | GCTCAT |
| 10 | R-ITR | AGGAACCCCTAGTGATGGAGTTGGCCACTCCCTCTCTGCGCGCTC |
| | sequence(G | GCTCGCTCACTGAGGCCGGGCGACCAAAGGTCGCCCGACGCCCGG |
| | OI-C59 | GCTTTGCCCGGGCGGCCTCAGTGAGCGAGCGAGCGCGCAGCTGCC |
| | Plasmid | TGCAGG |
| | expression | |
| | cassette) | |
| 11 | L-ITR | GGGCCACTCCCTCTCTGCGCGCTCGCTCGCTCACTGAGGCCGGGC |
| | sequence(G | GACCAAAGGTCGCCCGACGCCCGGGCTTTGCCCGGGCGGCCTCAG |
| | OI-C21 | TGAGCGAGCGAGCGCGCAGAGAGGGAGTGGCCAACTCCATCACTA |
| | Plasmid | GGGGTTCCT |
| | expression | |
| | cassette) | |
| 12 | SV40 | ATGCTTTATTTGTGAAATTTGTGATGCTATTGCTTTATTTGTAAC |
| | poly(A) | CATTATAAGCTGCAATAAACAAGTTAACAACAACAATTGCATTCA |
| | signal | TTTTATGTTTCAGGTTCAGGGGGAGGTGTGGGAGGTTTTTTAAA |
| | polyadenylation | |
| | region | |
| | (GOI-C21 | |
| | Plasmid | |
| | expression | |
| | cassette) | |
| 13 | R-ITR | CCACTCCCTCTCTGCGCGCTCGCTCGCTCACTGAGGCCGGGCGAC |
| | sequence(G | CAAAGGTCGCCCGACGCCCGGGCTTTGCCCGGGCGGCCTCAGTGA |
| | OI-C21 | GCGAGCGAGCGCGCAGAGAGGGA |
| | Plasmid | |
| | expression | |
| | cassette) | |
| 14 | GOI-C21 | AAAGCTTCCCGGGGGGATCTGGGCCACTCCCTCTCTGCGCGCTCG |
| | Plasmid | CTCGCTCACTGAGGCCGGGCGACCAAAGGTCGCCCGACGCCCGGG |
| | sequence | CTTTGCCCGGGCGGCCTCAGTGAGCGAGCGAGCGCGCAGAGAGGG |
| | | AGTGGCCAACTCCATCACTAGGGGTTCCTGGAGGGGTGGAGTCGT |
| | | GACCTAGGCGTTACATAACTTACGGTAAATGGCCCGCCTGGCTGA |
| | | CCGCCCAACGACCCCCGCCCATTGACGTCAATAATGACGTATGTT |
| | | CCCATAGTAACGCCAATAGGGACTTTCCATTGACGTCAATGGGTG |
| | | GACTATTTACGGTAAACTGCCCACTTGGCAGTACATCAAGTGTAT |
| | | CATATGCCAAGTACGCCCCCTATTGACGTCAATGACGGTAAATGG |
| | | CCCGCCTGGCATTATGCCCAGTACATGACCTTATGGGACTTTCCT |
| | | ACTTGGCAGTACATCTACGTATTAGTCATCGCTATTACCATGGGT |
| | | CGAGGTGAGCCCCACGTTCTGCTTCACTCTCCCCATCTCCCCCCC |
| | | CTCCCCACCCCCAATTTTGTATTTATTTATTTTTTAATTATTTTG |
| | | TGCAGCGATGGGGGCGGGGGGGGGGGGGGCGCGCGCCAGGCGGGG |
| | | CGGGGCGGGGCGAGGGGCGGGGCGGGGCGAGGCGGAGAGGTGCGG |
| | | CGGCAGCCAATCAGAGCGGCGCGCTCCGAAAGTTTCCTTTTATGG |
| | | CGAGGCGGCGGCGGCGGCGGCCCTATAAAAAGCGAAGCGCGCGGC |
| | | GGGCGGATATCGGAGTCGCTGCGTTGCCTTCGCCCCGTGCCCCGC |
| | | TCCGCGCCGCCTCGCGCCGCCCGCCCCGGCTCTGACTGACCGCGT |
| | | TACTCCCACAGGTGAGCGGGCGGGACGGCCCTTCTCCTCCGGGCT |
| | | GTAATTAGCGCTTGGTTTAATGACGGCTCGTTTCTTTTCTGTGGC |
| | | TGCGTGAAAGCCTTAAAGGGCTCCGGGAGGGCCCTTTGTGCGGGG |
| | | GGGAGCGGCTCGGGGGGTGCGTGCGTGTGTGTGTGCGTGGGGAGC |
| | | GCCGCGTGCGGCCCGCGCTGCCCGGCGGCTGTGAGCGCTGCGGGC |
| | | GCGGCGCGGGGCTTTGTGCGCTCCGCGTGTGCGCGAGGGGAGCGC |
| | | GGCCGGGGGCGGTGCCCCGCGGTGCGGGGGGGCTGCGAGGGGAAC |
| | | AAAGGCTGCGTGCGGGGTGTGTGCGTGGGGGGGTGAGCAGGGGGT |
| | | GTGGGCGCGGCGGTCGGGCTGTAACCCCCCCCTGCACCCCCCTCC |
| | | CCGAGTTGCTGAGCACGGCCCGGCTTCGGGTGCGGGGCTCCGTGC |
| | | GGGGCGTGGCGCGGGGCTCGCCGTGCCGGGCGGGGGGTGGCGGCA |
| | | GGTGGGGGTGCCGGGCGGGGCGGGGCCGCCTCGGGCCGGGGAGGG |
| | | CTCGGGGGAGGGGCGCGGCGGCCCCGGAGCGCCGGCGGCTGTCGA |
| | | GGCGCGGCGAGCCGCAGCCATTGCCTTTTATGGTAATCGTGCGAG |
| | | AGGGCGCAGGGACTTCCTTTGTCCCAAATCTGGCGGAGCCGAAAT |
| | | CTGGGAGGCGCCGCCGCACCCCCTCTAGCGGGCGCGGGCGAAGCG |
| | | GTGCGGCGCCGGCAGGAAGGAAATGGGCGGGGAGGGCCTTCGTGC |
| | | GTCGCCGCGCCGCCGTCCCCTTCTCCATCTCCAGCCTCGGGGCTG |
| | | CCGCAGGGGGACGGCTGCCTTCGGGGGGGACGGGGCAGGGCGGGG |
| | | TTCGGCTTCTGGCGTGTGACCGGCGGCTCTAGAGCCTCTGCTAAC |
| | | CATGTTCATGCCTTCTTCTTTTTCCTACAGACCGGTCTCCTGGGC |
| | | AACGTGCTGGTTATTGTGCTGTCTCATCATTTTGGCAAAGAATTC |
| | | CACCGCCACCATGAGCCGGAAGATCGAGGGCTTCCTGCTGCTGCT |
| | | GCTGTTTGGATACGAGGCCACCCTGGGACTGTCCTCCACCGAGGA |
| | | CGAGGGCGAGGACCCCTGGTACCAGAAGGCCTGCAAGTGCGACTG |
| | | CCAGGGGGGCCTAACGCCCTGTGGAGCGCTGGAGCTACCAGCCTG |
| | | GACTGCATTCCCGAGTGCCCATATCACAAGCCCCTGGGATTCGAG |
| | | AGCGGAGAGGTGACCCCAGACCAGATTACATGCAGTAACCCAGAA |
| | | CAGTATGTGGGATGGTATTCAAGCTGGACCGCCAACAAGGCCAGA |
| | | CTGAACAGCCAGGGCTTCGGCTGCGCCTGGCTGAGCAAGTTCCAG |
| | | GACAGCAGCCAGTGGCTGCAGATTGACCTGAAGGAGATCAAGGTG |
| | | ATCTCAGGGATCCTGACCCAGGGAAGATGCGACATCGATGAGTGG |
| | | ATGACCAAGTACAGCGTGCAGTACAGAACCGACGAAAGACTGAAC |
| | | TGGATCTACTACAAAGACCAGACAGGAAACAACAGAGTGTTCTAC |
| | | GGCAACAGCGACAGAACCAGCACCGTGCAGAACCTGCTGAGACCC |
| | | CCCATCATCAGCAGATTCATCAGACTGATCCCCCTGGGGTGGCAC |
| | | GTGAGAATCGCAATCAGAATGGAGCTGCTGGAATGCGTGAGCAAG |
| | | TGCGCCTAAAGCGGCCGCTAGGCCTCACCTGCGATCTCGATGCTT |
| | | TATTTGTGAAATTTGTGATGCTATTGCTTTATTTGTAACCATTAT |
| | | AAGCTGCAATAAACAAGTTAACAACAACAATTGCATTCATTTTAT |
| | | GTTTCAGGTTCAGGGGGAGGTGTGGGAGGTTTTTTAAACTAGTCC |
| | | ACTCCCTCTCTGCGCGCTCGCTCGCTCACTGAGGCCGGGCGACCA |
| | | AAGGTCGCCCGACGCCCGGGCTTTGCCCGGGCGGCCTCAGTGAGC |
| | | GAGCGAGCGCGCAGAGAGGGACAGATCCGGGCCCGCATGCGTCGA |
| | | CAATTCACTGGCCGTCGTTTTACAACGTCGTGACTGGGAAAACCC |
| | | TGGCGTTACCCAACTTAATCGCCTTGCAGCACATCCCCCTTTCGC |
| | | CAGCTGGCGTAATAGCGAAGAGGCCCGCACCGATCGCCCTTCCCA |
| | | ACAGTTGCGCAGCCTGAATGGCGAATGGCGCCTGATGCGGTATTT |
| | | TCTCCTTACGCATCTGTGCGGTATTTCACACCGCATATGGTGCAC |
| | | TCTCAGTACAATCTGCTCTGATGCCGCATAGTTAAGCCAGCCCCG |
| | | ACACCCGCCAACACCCGCTGACGCGCCCTGACGGGCTTGTCTGCT |
| | | CCCGGCATCCGCTTACAGACAAGCTGTGACCGTCTCCGGGAGCTG |
| | | CATGTGTCAGAGGTTTTCACCGTCATCACCGAAACGCGCGAGACG |
| | | AAAGGGCCTCGTGATACGCCTATTTTTATAGGTTAATGTCATGAT |
| | | AATAATGGTTTCTTAGACGTCAGGTGGCACTTTTCGGGGAAATGT |
| | | GCGCGGAACCCCTATTTGTTTATTTTTCTAAATACATTCAAATAT |
| | | GTATCCGCTCATGAGACAATAACCCTGATAAATGCTTCAATAATA |
| | | TTGAAAAAGGAAGAGTATGAGTATTCAACATTTCCGTGTCGCCCT |
| | | TATTCCCTTTTTTGCGGCATTTTGCCTTCCTGTTTTTGCTCACCC |
| | | AGAAACGCTGGTGAAAGTAAAAGATGCTGAAGATCAGTTGGGTGC |
| | | ACGAGTGGGTTACATCGAACTGGATCTCAACAGCGGTAAGATCCT |
| | | TGAGAGTTTTCGCCCCGAAGAACGTTTTCCAATGATGAGCACTTT |
| | | TAAAGTTCTGCTATGTGGCGCGGTATTATCCCGTATTGACGCCGG |
| | | GCAAGAGCAACTCGGTCGCCGCATACACTATTCTCAGAATGACTT |
| | | GGTTGAGTACTCACCAGTCACAGAAAAGCATCTTACGGATGGCAT |
| | | GACAGTAAGAGAATTATGCAGTGCTGCCATAACCATGAGTGATAA |
| | | CACTGCGGCCAACTTACTTCTGACAACGATCGGAGGACCGAAGGA |
| | | GCTAACCGCTTTTTTGCACAACATGGGGGATCATGTAACTCGCCT |
| | | TGATCGTTGGGAACCGGAGCTGAATGAAGCCATACCAAACGACGA |
| | | GCGTGACACCACGATGCCTGTAGCAATGGCAACAACGTTGCGCAA |
| | | ACTATTAACTGGCGAACTACTTACTCTAGCTTCCCGGCAACAATT |
| | | AATAGACTGGATGGAGGCGGATAAAGTTGCAGGACCACTTCTGCG |
| | | CTCGGCCCTTCCGGCTGGCTGGTTTATTGCTGATAAATCTGGAGC |
| | | CGGTGAGCGTGGGTCTCGCGGTATCATTGCAGCACTGGGGCCAGA |
| | | TGGTAAGCCCTCCCGTATCGTAGTTATCTACACGACGGGGAGTCA |
| | | GGCAACTATGGATGAACGAAATAGACAGATCGCTGAGATAGGTGC |
| | | CTCACTGATTAAGCATTGGTAACTGTCAGACCAAGTTTACTCATA |
| | | TATACTTTAGATTGATTTAAAACTTCATTTTTAATTTAAAAGGAT |
| | | CTAGGTGAAGATCCTTTTTGATAATCTCATGACCAAAATCCCTTA |
| | | ACGTGAGTTTTCGTTCCACTGAGCGTCAGACCCCGTAGAAAAGAT |
| | | CAAAGGATCTTCTTGAGATCCTTTTTTTCTGCGCGTAATCTGCTG |
| | | CTTGCAAACAAAAAAACCACCGCTACCAGCGGTGGTTTGTTTGCC |
| | | GGATCAAGAGCTACCAACTCTTTTTCCGAAGGTAACTGGCTTCAG |
| | | CAGAGCGCAGATACCAAATACTGTTCTTCTAGTGTAGCCGTAGTT |
| | | AGGCCACCACTTCAAGAACTCTGTAGCACCGCCTACATACCTCGC |
| | | TCTGCTAATCCTGTTACCAGTGGCTGCTGCCAGTGGCGATAAGTC |
| | | GTGTCTTACCGGGTTGGACTCAAGACGATAGTTACCGGATAAGGC |
| | | GCAGCGGTCGGGCTGAACGGGGGGTTCGTGCACACAGCCCAGCTT |
| | | GGAGCGAACGACCTACACCGAACTGAGATACCTACAGCGTGAGCT |
| | | ATGAGAAAGCGCCACGCTTCCCGAAGGGAGAAAGGCGGACAGGTA |
| | | TCCGGTAAGCGGCAGGGTCGGAACAGGAGAGCGCACGAGGGAGCT |
| | | TCCAGGGGGAAACGCCTGGTATCTTTATAGTCCTGTCGGGTTTCG |
| | | CCACCTCTGACTTGAGCGTCGATTTTTGTGATGCTCGTCAGGGGG |
| | | GCGGAGCCTATGGAAAAACGCCAGCAACGCGGCCTTTTTACGGTT |
| | | CCTGGCCTTTTGCTGGCCTTTTGCTCACATGTTCTTTCCTGCGTT |
| | | ATCCCCTGATTCTGTGGATAACCGTATTACCGCCTTTGAGTGAGC |
| | | TGATACCGCTCGCCGCAGCCGAACGACCGAGCGCAGCGAGTCAGT |
| | | GAGCGAGGAAGCGGAAGAGCGCCCAATACGCAAACCGCCTCTCCC |
| | | CGCGCGTTGGCCGATTCATTAATGCAGCTGGCACGACAGGTTTCC |
| | | CGACTGGAAAGCGGGCAGTGAGCGCAACGCAATTAATGTGAGTTA |
| | | GCTCACTCATTAGGCACCCCAGGCTTTACACTTTATGCTTCCGGC |
| | | TCGTATGTTGTGTGGAATTGTGAGCGGATAACAATTTCACACAGG |
| | | AAACAGCTATGACCATGATTACGCCAAGCTCTCGAGATCTAG |
| 15 | RS1 amino | MSRKIEGFLLLLLFGYEATLGLSSTEDEGEDPWYQKACKCDCQGG |
| | acid | PNALWSAGATSLDCIPECPYHKPLGFESGEVTPDQITCSNPEQYV |
| | sequence | GWYSSWTANKARLNSQGFGCAWLSKFQDSSQWLQIDLKEIKVISG |
| | | ILTQGRCDIDEWMTKYSVQYRTDERLNWIYYKDQTGNNRVFYGNS |
| | | DRTSTVQNLLRPPIISRFIRLIPLGWHVRIAIRMELLECVSKCA |
| 16 | RS1 | ATGAGCCGGAAGATCGAGGGCTTCCTGCTGCTGCTGCTGTTTGGA |
| | Codon- | TACGAGGCCACCCTGGGACTGTCCTCCACCGAGGACGAGGGCGAG |
| | optimized | GACCCCTGGTACCAGAAGGCCTGCAAGTGCGACTGCCAGGGCGGG |
| | base | CCTAACGCCCTGTGGAGCGCTGGAGCTACCAGCCTGGACTGCATT |
| | sequence | CCCGAGTGCCCATATCACAAGCCCCTGGGATTCGAGAGCGGAGAG |
| | 1 (hRS1-1) | GTGACCCCAGACCAGATTACATGCAGTAACCCAGAACAGTATGTG |
| | | GGATGGTATTCAAGCTGGACCGCCAACAAGGCCAGACTGAACAGC |
| | | CAGGGCTTCGGCTGCGCCTGGCTGAGCAAGTTCCAGGACAGCAGC |
| | | CAGTGGCTGCAGATTGACCTGAAGGAGATCAAGGTGATCTCAGGG |
| | | ATCCTGACCCAGGGAAGATGCGACATCGATGAGTGGATGACCAAG |
| | | TACAGCGTGCAGTACAGAACCGACGAAAGACTGAACTGGATCTAC |
| | | TACAAAGACCAGACAGGAAACAACAGAGTGTTCTACGGCAACAGC |
| | | GACAGAACCAGCACCGTGCAGAACCTGCTGAGACCCCCCATCATC |
| | | AGCAGATTCATCAGACTGATCCCCCTGGGGTGGCACGTGAGAATC |
| | | GCAATCAGAATGGAGCTGCTGGAATGCGTGAGCAAGTGCGCCTGA |
| 17 | RS1 | ATGTCCAGGAAAATCGAAGGATTCCTGCTGCTGCTGCTGTTCGGG |
| | Codon- | TACGAGGCCACCCTGGGACTGTCCTCCACCGAGGATGAGGGCGAA |
| | optimized | GACCCATGGTATCAGAAGGCCTGCAAATGCGACTGTCAGGGCGGC |
| | gene | CCCAACGCCCTGTGGTCAGCCGGCGCTACCAGCCTGGACTGCATC |
| | sequence 2 | CCAGAGTGCCCCTACCACAAGCCCCTGGGATTCGAATCCGGCGAG |
| | (hRS1-2) | GTGACCCCAGACCAGATTACCTGTTCCAATCCCGAGCAGTATGTG |
| | | GGGTGGTATTCCAGCTGGACCGCCAACAAGGCCAGGCTGAACTCA |
| | | CAGGGGTTCGGCTGCGCCTGGCTGAGCAAGTTCCAGGATTCCTCC |
| | | CAGTGGCTGCAGATTGACCTGAAGGAGATCAAGGTGATCTCCGGC |
| | | ATCCTGACCCAGGGGAGGTGTGACATCGACGAATGGATGACCAAG |
| | | TACAGCGTGCAGTACAGAACCGACGAGAGGCTGAACTGGATCTAT |
| | | TATAAGGATCAGACAGGAAACAACCGCGTGTTCTACGGTAACTCC |
| | | GATAGAACAAGCACAGTGCAGAACCTGCTGCGGCCTCCAATCATC |
| | | AGTCGATTTATTCGGCTGATCCCCCTGGGCTGGCACGTGCGCATC |
| | | GCCATTAGGATGGAGCTGCTGGAGTGTGTGAGCAAGTGCGCCTGA |
| 18 | RS1 | ATGTACAGAATGCAGCTGCTGAGCTGCATTGCCCTGAGCCTGGCC |
| | Codon-opti | CTGGTGACAAACTCCCTGGGCCTGTCCAGCACAGAGGATGAGGGA |
| | mized gene | GAGGACCCTTGGTATCAGAAGGCCTGCAAATGCGACTGCCAGGGC |
| | sequence 3 | GGGCCAAATGCTCTGTGGAGTGCCGGCGCCACCTCCCTGGACTGT |
| | (hRS1-3) | ATCCCCGAGTGTCCCTACCACAAGCCCCTGGGCTTCGAGAGCGGC |
| | | GAGGTCACCCCTGATCAGATAACTTGCTCCAACCCCGAGCAGTAT |
| | | GTGGGCTGGTATAGCAGCTGGACCGCCAACAAAGCCCGGCTGAAC |
| | | AGCCAGGGCTTCGGCTGCGCATGGCTGAGCAAGTTCCAGGACAGC |
| | | AGCCAGTGGCTGCAGATCGACCTGAAAGAGATCAAGGTGATCTCC |
| | | GGCATCCTGACCCAGGGCCGGTGCGACATTGACGAGTGGATGACC |
| | | AAGTACTCCGTGCAGTATCGCACCGATGAGCGCCTGAATTGGATC |
| | | TATTATAAAGACCAGACCGGAAACAACAGAGTGTTTTACGGAAAC |
| | | AGCGACCGGACCAGCACTGTGCAGAACCTGCTGCGCCCTCCGATC |
| | | ATTTCTCGGTTTATCAGGCTGATTCCCCTGGGCTGGCACGTCCGG |
| | | ATCGCCATCCGCATGGAACTGCTGGAGTGTGTGAGTAAGTGTGCC |
| | | TGA |
| 19 | AAV2.7m8 | MAADGYLPDWLEDTLSEGIRQWWKLKPGPPPPKPAERHKDDSRGL |
| | VP1 amino | VLPGYKYLGPFNGLDKGEPVNEADAAALEHDKAYDRQLDSGDNPY |
| | acid | LKYNHADAEFQERLKEDTSFGGNLGRAVFQAKKRVLEPLGLVEEP |
| | sequence | VKTAPGKKRPVEHSPVEPDSSSGTGKAGQQPARKRLNFGQTGDAD |
| | | SVPDPQPLGQPPAAPSGLGTNTMATGSGAPMADNNEGADGVGNSS |
| | | GNWHCDSTWMGDRVITTSTRTWALPTYNNHLYKQISSQSGASNDN |
| | | HYFGYSTPWGYFDFNRFHCHFSPRDWQRLINNNWGFRPKRLNFKL |
| | | FNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRT |
| | | GNNFTFSYTFEDVPFHSSYAHSQSLDRLMNPLIDQYLYYLSRTNT |
| | | PSGTTTQSRLQFSQAGASDIRDQSRNWLPGPCYRQQRVSKTSADN |
| | | NNSEYSWTGATKYHLNGRDSLVNPGPAMASHKDDEEKFFPQSGVL |
| | | IFGKQGSEKTNVDIEKVMITDEEEIRTTNPVATEQYGSVSTNLQR |
| | | GNLALGETTRPARQAATADVNTQGVLPGMVWQDRDVYLQGPIWAK |
| | | IPHTDGHFHPSPLMGGFGLKHPPPQILIKNTPVPANPSTTFSAAK |
| | | FASFITQYSTGQVSVEIEWELQKENSKRWNPEIQYTSNYNKSVNV |
| | | DFTVDTNGVYSEPRPIGTRYLTRNL* |
| 20 | RS1 | ATGTCACGCAAGATAGAAGGCTTTTTGTTATTACTTCTCTTTGGC |
| | Wild-type | TATGAAGCCACATTGGGATTATCGTCTACCGAGGATGAAGGCGAG |
| | coding gene | GACCCCTGGTACCAAAAAGCATGCAAGTGCGATTGCCAAGGAGGA |
| | sequence | CCCAATGCTCTGTGGTCTGCAGGTGCCACCTCCTTGGACTGTATA |
| | hRS1-WT | CCAGAATGCCCATATCACAAGCCTCTGGGTTTCGAGTCAGGGGAG |
| | 675 bp | GTCACACCGGACCAGATCACCTGCTCTAACCCGGAGCAGTATGTG |
| | | GGCTGGTATTCTTCGTGGACTGCAAACAAGGCCCGGCTCAACAGT |
| | | CAAGGCTTTGGGTGTGCCTGGCTCTCCAAGTTCCAGGACAGTAGC |
| | | CAGTGGTTACAGATAGATCTGAAGGAGATCAAAGTGATTTCAGGG |
| | | ATCCTCACCCAGGGGCGCTGTGACATCGATGAGTGGATGACCAAG |
| | | TACAGCGTGCAGTACAGGACCGATGAGCGCCTGAACTGGATTTAC |
| | | TACAAGGACCAGACTGGAAACAACCGGGTCTTCTATGGCAACTCG |
| | | GACCGCACCTCCACGGTTCAGAACCTGCTGCGGCCCCCCATCATC |
| | | TCCCGCTTCATCCGCCTCATCCCGCTGGGCTGGCACGTCCGCATT |
| | | GCCATCCGGATGGAGCTGCTGGAGTGCGTCAGCAAGTGTGCCTGA |
| 21 | GOI-E04 | AGCGCCCAATACGCAAACCGCCTCTCCCCGCGCGTTGGCCGATTC |
| | Plasmid | ATTAATGCAGCTGGCACGACAGGTTTCCCGACTGGAAAGCGGGCA |
| | backbone | GTGAGCGCAACGCAATTAATGTGAGTTAGCTCACTCATTAGGCAC |
| | sequence | CCCAGGCTTTACACTTTATGCTTCCGGCTCGTATGTTGTGTGGAA |
| | | TTGTGAGCGGATAACAATTTCACACAGGAAACAGCTATGACCATG |
| | | ATTACGAATTGCCTGCAGGCAGCTGCGCGCTCGCTCGCTCACTGA |
| | | GGCCGCCCGGGCAAAGCCCGGGCGTCGGGCGACCTTTGGTCGCCC |
| | | GGCCTCAGTGAGCGAGCGAGCGCGCAGAGAGGGAGTGGCCAACTC |
| | | CATCACTAGGGGTTCCTATCGATATCAAGCTTCGTTACATAACTT |
| | | ACGGTAAATGGCCCGCCTGGCTGACCGCCCAACGACCCCCGCCCA |
| | | TTGACGTCAATAATGACGTATGTTCCCATAGTAACGCCAATAGGG |
| | | ACTTTCCATTGACGTCAATGGGTGGACTATTTACGGTAAACTGCC |
| | | CACTTGGCAGTACATCAAGTGTATCATATGCCAAGTACGCCCCCT |
| | | ATTGACGTCAATGACGGTAAATGGCCCGCCTGGCATTATGCCCAG |
| | | TACATGACCTTATGGGACTTTCCTACTTGGCAGTACATCTACGTA |
| | | TTAGTCATCGCTATTACCATGGGTCGAGGTGAGCCCCACGTTCTG |
| | | CTTCACTCTCCCCATCTCCCCCCCCTCCCCACCCCCAATTTTGTA |
| | | TTTATTTATTTTTTAATTATTTTGTGCAGCGATGGGGGCGGGGGG |
| | | GGGGGGGGCGCGCGCCAGGCGGGGCGGGGGGGGGCGAGGGGCGGG |
| | | GCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGC |
| | | CCTATAAAAAGCGAAGCGCGCGGCGGGCGGATATCGGAGTCGCTG |
| | | CGTTGCCTTCGCCCCGTGCCCCGCTCCGCGCCGCCTCGCGCCGCC |
| | | CGCCCCGGCTCTGACTGACCGCGTTACTCCCACAGGTGAGCGGGC |
| | | GGGACGGCCCTTCTCCTCCGGGCTGTAATTAGCGCTTGGTTTAAT |
| | | GACGGCTCGTTTCTTTTCTGTGGCTGCGTGAAAGCCTTAAAGGGC |
| | | TCCGGGAGGGCCCTTTGTGCGGGGGGGAGCGGCTCGGGGGGTGCG |
| | | TGCGTGTGTGTGTGCGTGGGGAGCGCCGCGTGCGGCCCGCGCTGC |
| | | CCGGCGGCTGTGAGCGCTGCGGGCGCGGCGCGGGGCTTTGTGCGC |
| | | TCCGCGTGTGCGCGAGGGGAGCGCGGCCGGGGGCGGTGCCCCGCG |
| | | GTGCGGGGGGGCTGCGAGGGGAACAAAGGCTGCGTGCGGGGTGTG |
| | | TGCGTGGGGGGGTGAGCAGGGGGTGTGGGCGCGGCGGTCGGGCTG |
| | | TAACCCCCCCCTGCACCCCCCTCCCCGAGTTGCTGAGCACGGCCC |
| | | GGCTTCGGGTGCGGGGCTCCGTGCGGGGCGTGGCGCGGGGCTCGC |
| | | CGTGCCGGGCGGGGGGTGGCGGCAGGTGGGGGTGCCGGGCGGGGC |
| | | GGGGCCGCCTCGGGCCGGGGAGGGCTCGGGGGAGGGGCGCGGCGG |
| | | CCCCGGAGCGCCGGCGGCTGTCGAGGCGCGGCGAGCCGCAGCCAT |
| | | TGCCTTTTATGGTAATCGTGCGAGAGGGCGCAGGGACTTCCTTTG |
| | | TCCCAAATCTGGCGGAGCCGAAATCTGGGAGGCGCCGCCGCACCC |
| | | CCTCTAGCGGGCGCGGGCGAAGCGGTGCGGCGCCGGCAGGAAGGA |
| | | AATGGGCGGGGAGGGCCTTCGTGCGTCGCCGCGCCGCCGTCCCCT |
| | | TCTCCATCTCCAGCCTCGGGGCTGCCGCAGGGGGACGGCTGCCTT |
| | | CGGGGGGGACGGGGCAGGGCGGGGTTCGGCTTCTGGCGTGTGACC |
| | | GGCGGCTCTAGAGCCTCTGCTAACCATGTTCATGCCTTCTTCTTT |
| | | TTCCTACAGGGGCCCAGCCGGCCTCGCGAGAATTCTCTAGAACGC |
| | | CGCCatggtgagcaagggcgaggagctgttcaccggggtggtgcc |
| | | catcctggtcgagctggacggcgacgtaaacggccacaagttcag |
| | | cgtgtccggcgagggcgagggcgatgccacctacggcaagctgac |
| | | cctgaagttcatctgcaccaccggcaagctgcccgtgccctggcc |
| | | caccctcgtgaccaccctgacctacggcgtgcagtgcttcagccg |
| | | ctaccccgaccacatgaagcagcacgacttcttcaagtccgccat |
| | | gcccgaaggctacgtccaggagcgcaccatcttcttcaaggacga |
| | | cggcaactacaagacccgcgccgaggtgaagttcgagggcgacac |
| | | cctggtgaaccgcatcgagctgaagggcatcgacttcaaggagga |
| | | cggcaacatcctggggcacaagctggagtacaactacaacagcca |
| | | caacgtctatatcatggccgacaagcagaagaacggcatcaaggt |
| | | gaacttcaagatccgccacaacatcgaggacggcagcgtgcagct |
| | | cgccgaccactaccagcagaacacccccatcggcgacggccccgt |
| | | gctgctgcccgacaaccactacctgagcacccagtccgccctgag |
| | | caaagaccccaacgagaagcgcgatcacatggtcctgctggagtt |
| | | cgtgaccgccgccgggatcactctcggcatggacgagctgtacaa |
| | | gTAGAGCTgacactagtgcGGATCCACGGGTGGCATCCCTGTGAC |
| | | CCCTCCCCAGTGCCTCTCCTGGCCCTGGAAGTTGCCACTCCAGTG |
| | | CCCACCAGCCTTGTCCTAATAAAATTAAGTTGCATCATTTTGTCT |
| | | GACTAGGTGTCCTTCTATAATATTATGGGGTGGAGGGGGGTGGTA |
| | | TGGAGCAAGGGGCAAGTTGGGAAGACAACCTGTAGGGCCTGCGGG |
| | | GTCTATTGGGAACCAAGCTGGAGTGCAGTGGCACAATCTTGGCTC |
| | | ACTGCAATCTCCGCCTCCTGGGTTCAAGCGATTCTCCTGCCTCAG |
| | | CCTCCCGAGTTGTTGGGATTCCAGGCATGCATGACCAGGCTCAGC |
| | | TAATTTTTGTTTTTTTGGTAGAGACGGGGTTTCACCATATTGGCC |
| | | AGGCTGGTCTCCAACTCCTAATCTCAGGTGATCTACCCACCTTGG |
| | | CCTCCCAAATTGCTGGGATTACAGGCGTGAACCACTGCTCCCTTC |
| | | CCTGTCCTTATCGATAGATCTAGGAACCCCTAGTGATGGAGTTGG |
| | | CCACTCCCTCTCTGCGCGCTCGCTCGCTCACTGAGGCCGGGCGAC |
| | | CAAAGGTCGCCCGACGCCCGGGCTTTGCCCGGGCGGCCTCAGTGA |
| | | GCGAGCGAGCGCGCAGCTGCCTGCAGGCAGCTTGGCACTGGCCGT |
| | | CGTTTTACAACGTCGTGACTGGGAAAACCCTGGCGTTACCCAACT |
| | | TAATCGCCTTGCAGCACATCCCCCTTTCGCCAGCTGGCGTAATAG |
| | | CGAAGAGGCCCGCACCGATCGCCCTTCCCAACAGTTGCGCAGCCT |
| | | GAATGGCGAATGGCGCCTGATGCGGTATTTTCTCCTTACGCATCT |
| | | GTGCGGTATTTCACACCGCATACGTCAAAGCAACCATAGTACGCG |
| | | CCCTGTAGCGGCGCATTAAGCGCGGCGGGTGTGGTGGTTACGCGC |
| | | AGCGTGACCGCTACACTTGCCAGCGCCCTAGCGCCCGCTCCTTTC |
| | | GCTTTCTTCCCTTCCTTTCTCGCCACGTTCGCCGGCTTTCCCCGT |
| | | CAAGCTCTAAATCGGGGGCTCCCTTTAGGGTTCCGATTTAGTGCT |
| | | TTACGGCACCTCGACCCCAAAAAACTTGATTTGGGTGATGGTTCA |
| | | CGTAGTGGGCCATCGCCCTGATAGACGGTTTTTCGCCCTTTGACG |
| | | TTGGAGTCCACGTTCTTTAATAGTGGACTCTTGTTCCAAACTGGA |
| | | ACAACACTCAACCCTATCTCGGGCTATTCTTTTGATTTATAAGGG |
| | | ATTTTGCCGATTTCGGCCTATTGGTTAAAAAATGAGCTGATTTAA |
| | | CAAAAATTTAACGCGAATTTTAACAAAATATTAACGTTTACAATT |
| | | TTATGGTGCACTCTCAGTACAATCTGCTCTGATGCCGCATAGTTA |
| | | AGCCAGCCCCGACACCCGCCAACACCCGCTGACGCGCCCTGACGG |
| | | GCTTGTCTGCTCCCGGCATCCGCTTACAGACAAGCTGTGACCGTC |
| | | TCCGGGAGCTGCATGTGTCAGAGGTTTTCACCGTCATCACCGAAA |
| | | CGCGCGAGACGAAAGGGCCTCGTGATACGCCTATTTTTATAGGTT |
| | | AATGTCATGATAATAATGGTTTCTTAGACGTCAGGTGGCACTTTT |
| | | CGGGGAAATGTGCGGAACCCCTATTTGTTTATTTTTCTAAATACA |
| | | TTCAAATATGTATCCGCTCATGAATTAATTCTTAGAAAAACTCAT |
| | | CGAGCATCAAATGAAACTGCAATTTATTCATATCAGGATTATCAA |
| | | TACCATATTTTTGAAAAAGCCGTTTCTGTAATGAAGGAGAAAACT |
| | | CACCGAGGCAGTTCCATAGGATGGCAAGATCCTGGTATCGGTCTG |
| | | CGATTCCGACTCGTCCAACATCAATACAACCTATTAATTTCCCCT |
| | | CGTCAAAAATAAGGTTATCAAGTGAGAAATCACCATGAGTGACGA |
| | | CTGAATCCGGTGAGAATGGCAAAAGTTTATGCATTTCTTTCCAGA |
| | | CTTGTTCAACAGGCCAGCCATTACGCTCGTCATCAAAATCACTCG |
| | | CATCAACCAAACCGTTATTCATTCGTGATTGCGCCTGAGCGAGAC |
| | | GAAATACGCGATCGCTGTTAAAAGGACAATTACAAACAGGAATCG |
| | | AATGCAACCGGCGCAGGAACACTGCCAGCGCATCAACAATATTTT |
| | | CACCTGAATCAGGATATTCTTCTAATACCTGGAATGCTGTTTTCC |
| | | CGGGGATCGCAGTGGTGAGTAACCATGCATCATCAGGAGTACGGA |
| | | TAAAATGCTTGATGGTCGGAAGAGGCATAAATTCCGTCAGCCAGT |
| | | TTAGTCTGACCATCTCATCTGTAACATCATTGGCAACGCTACCTT |
| | | TGCCATGTTTCAGAAACAACTCTGGCGCATCGGGCTTCCCATACA |
| | | ATCGATAGATTGTCGCACCTGATTGCCCGACATTATCGCGAGCCC |
| | | ATTTATACCCATATAAATCAGCATCCATGTTGGAATTTAATCGCG |
| | | GCCTAGAGCAAGACGTTTCCCGTTGAATATGGCTCATAACACCCC |
| | | TTGTATTACTGTTTATGTAAGCAGACAGTTTTATTGTTCATGACC |
| | | AAAATCCCTTAACGTGAGTTTTCGTTCCACTGAGCGTCAGACCCC |
| | | GTAGAAAAGATCAAAGGATCTTCTTGAGATCCTTTTTTTCTGCGC |
| | | GTAATCTGCTGCTTGCAAACAAAAAAACCACCGCTACCAGCGGTG |
| | | GTTTGTTTGCCGGATCAAGAGCTACCAACTCTTTTTCCGAAGGTA |
| | | ACTGGCTTCAGCAGAGCGCAGATACCAAATACTGTTCTTCTAGTG |
| | | TAGCCGTAGTTAGGCCACCACTTCAAGAACTCTGTAGCACCGCCT |
| | | ACATACCTCGCTCTGCTAATCCTGTTACCAGTGGCTGCTGCCAGT |
| | | GGCGATAAGTCGTGTCTTACCGGGTTGGACTCAAGACGATAGTTA |
| | | CCGGATAAGGCGCAGCGGTCGGGCTGAACGGGGGGTTCGTGCACA |
| | | CAGCCCAGCTTGGAGCGAACGACCTACACCGAACTGAGATACCTA |
| | | CAGCGTGAGCTATGAGAAAGCGCCACGCTTCCCGAAGGGAGAAAG |
| | | GCGGACAGGTATCCGGTAAGCGGCAGGGTCGGAACAGGAGAGCGC |
| | | ACGAGGGAGCTTCCAGGGGGAAACGCCTGGTATCTTTATAGTCCT |
| | | GTCGGGTTTCGCCACCTCTGACTTGAGCGTCGATTTTTGTGATGC |
| | | TCGTCAGGGGGGCGGAGCCTATGGAAAAACGCCAGCAACGCGGCC |
| | | TTTTTACGGTTCCTGGCCTTTTGCTGGCCTTTTGCTCACATGTTC |
| | | TTTCCTGCGTTATCCCCTGATTCTGTGGATAACCGTATTACCGCC |
| | | TTTGAGTGAGCTGATACCGCTCGCCGCAGCCGAACGACCGAGCGC |
| | | AGCGAGTCAGTGAGCGAGGAAGCGGAAG |
| 22 | EF1n | Gggcagagcgcacatcgcccacagtccccgagaagttggggggag |
| | promoter | gggtcggcaattgaaccggtgcctagagaaggtggcgcggggtaa |
| | | actgggaaagtgatgtcgtgtactggctccgcctttttcccgagg |
| | | gtgggggagaaccgtatataagtgcagtagtcgccgtgaacgttc |
| | | tttttcgcaacgggtttgccgccagaacacagctgaagcttcgag |
| | | gggctcgcatctctccttcacgcgcccgccgccctacctgaggcc |
| | | gccatccacgccggttgagtcgcgttctgccgcctcccgcctgtg |
| | | gtgcctcctgaactgcgtccgccgtctaggtaagtttaaagctca |
| | | ggtcgagaccgggcctttgtccggcgctcccttggagcctaccta |
| | | gactcagccggctctccacgctttgcctgaccctgcttgctcaac |
| | | tctacgtctttgtttcgttttctgttctgcgccgttacagatc |
| 23 | EF1n-Intron | Gggcagagcgcacatcgcccacagtccccgagaagttggggggag |
| | (EF1n | gggtcggcaattgaaccggtgcctagagaaggtggcgcggggtaa |
| | promoter | actgggaaagtgatgtcgtgtactggctccgcctttttcccgagg |
| | Intron) | gtgggggagaaccgtatataagtgcagtagtcgccgtgaacgttc |
| | | tttttcgcaacgggtttgccgccagaacacagctgaagcttcgag |
| | | gggctcgcatctctccttcacgcgcccgccgccctacctgaggcc |
| | | gccatccacgccggttgagtcgcgttctgccgcctcccgcctgtg |
| | | gtgcctcctgaactgcgtccgccgtctaggtaagtttaaagctca |
| | | ggtcgagaccgggcctttgtccggcgctcccttggagcctaccta |
| | | gactcagccggctctccacgctttgcctgaccctgcttgctcaac |
| | | tctacgtctttgtttcgttttctgttctgcgccgttacagatcca |
| | | agctgtgaccggcgcctacGATATCcgtttagtgaaccgtcagat |
| | | cgcctggagacgccatccacgctgttttgacctccatagaagaca |
| | | ccgggaccgatccagcctccgcggccgggaacggtgcattggaac |
| | | gcggattccccgtgccaagagtgacgtaagtaccgcctatagagt |
| | | ctataggcccacccccttggcttcgttagaacgcggctacaatta |
| | | atacataaccttatgtatcatacacatacgatttaggtgacacta |
| | | tagaataacatccactttgcctttctctccacaggtgtccactcc |
| | | caggtccaactgcacctcggttctaagcttctgcaggtcgactct |
| | | agaggatccACCGGTCTCCTGGGCAACGTGCTGGTTATTGTGCTG |
| | | TCTCATCATTTTGGCAAAGAATTC |
| 24 | Intron in | caagctgtgaccggcgcctacGATATCcgtttagtgaaccgtcag |
| | EF1n-Intron | atcgcctggagacgccatccacgctgttttgacctccatagaaga |
| | (nIntron) | caccgggaccgatccagcctccgcggccgggaacggtgcattgga |
| | | acgcggattccccgtgccaagagtgacgtaagtaccgcctataga |
| | | gtctataggcccacccccttggcttcgttagaacgcggctacaat |
| | | taatacataaccttatgtatcatacacatacgatttaggtgacac |
| | | tatagaataacatccactttgcctttctctccacaggtgtccact |
| | | cccaggtccaactgcacctcggttctaagcttctgcaggtcgact |
| | | ctagaggatccACCGGTCTCCTGGGCAACGTGCTGGTTATTGTGC |
| | | TGTCTCATCATTTTGGCAAAGAATTC |
| 25 | LTR-EF1n | ggggctcgcatctctccttcacgcgcccgccgccctacctgaggc |
| | Intron | cgccatccacgccggttgagtcgcgttctgccgcctcccgcctgt |
| | (LTR-nIntr | ggtgcctcctgaactgcgtccgccgtctaggtaagtttaaagctc |
| | on) (This | aggtcgagaccgggcctttgtccggcgctcccttggagcctacct |
| | sequence | agactcagccggctctccacgctttgcctgaccctgcttgctcaa |
| | comprises | ctctacgtctttgtttcgttttctgttctgcgccgttacagatcc |
| | Intron of | aagctgtgaccggcgcctacGATATCcgtttagtgaaccgtcaga |
| | EF1n) | tcgcctggagacgccatccacgctgttttgacctccatagaagac |
| | | accgggaccgatccagcctccgcggccgggaacggtgcattggaa |
| | | cgcggattccccgtgccaagagtgacgtaagtaccgcctatagag |
| | | tctataggcccacccccttggcttcgttagaacgcggctacaatt |
| | | aatacataaccttatgtatcatacacatacgatttaggtgacact |
| | | atagaataacatccactttgcctttctctccacaggtgtccactc |
| | | ccaggtccaactgcaccteggttctaagcttctgcaggtcgactc |
| | | tagaggatcc |
| 26 | Ubc | GGCCTCCGCGCCGGGTTTTGGCGCCTCCCGCGGGCGCCCCCCTCC |
| | promoter | TCACGGCGAGCGCTGCCACGTCAGACGAAGGGCGCAGCGAGCGTC |
| | | CTGATCCTTCCGCCCGGACGCTCAGGACAGCGGCCCGCTGCTCAT |
| | | AAGACTCGGCCTTAGAACCCCAGTATCAGCAGAAGGACATTTTAG |
| | | GACGGGACTTGGGTGACTCTAGGGCACTGGTTTTCTTTCCAGAGA |
| | | GCGGAACAGGCGAGGAAAAGTAGTCCCTTCTCGGCGATTCTGCGG |
| | | AGGGATCTCCGTGGGGCGGTGAACGCCGATGATTATATAAGGACG |
| | | CGCCGGGTGTGGCACAGCTAGTTCCGTCGCAGCCGGGATTTGGGT |
| | | CGCGGTTCTTGTTTGTGGATCGCTGTGATCGTCACTTGGT |
| 27 | Ubc-Intron | GGCCTCCGCGCCGGGTTTTGGCGCCTCCCGCGGGCGCCCCCCTCC |
| | (Ubc | TCACGGCGAGCGCTGCCACGTCAGACGAAGGGCGCAGCGAGCGTC |
| | promoter | CTGATCCTTCCGCCCGGACGCTCAGGACAGCGGCCCGCTGCTCAT |
| | intron) | AAGACTCGGCCTTAGAACCCCAGTATCAGCAGAAGGACATTTTAG |
| | | GACGGGACTTGGGTGACTCTAGGGCACTGGTTTTCTTTCCAGAGA |
| | | GCGGAACAGGCGAGGAAAAGTAGTCCCTTCTCGGCGATTCTGCGG |
| | | AGGGATCTCCGTGGGGCGGTGAACGCCGATGATTATATAAGGACG |
| | | CGCCGGGTGTGGCACAGCTAGTTCCGTCGCAGCCGGGATTTGGGT |
| | | CGCGGTTCTTGTTTGTGGATCGCTGTGATCGTCACTTGGTGAGTA |
| | | GCGGGCTGCTGGGCTGGCCGGGGCTTTCGTGGCCGCCGGGCCGCT |
| | | CGGTGGGACGGAAGCGTGTGGAGAGACCGCCAAGGGCTGTAGTCT |
| | | GGGTCCGCGAGCAAGGTTGCCCTGAACTGGGGGTTGGGGGGAGCG |
| | | CAGCAAAATGGCGGCTGTTCCCGAGTCTTGAATAGAACCTTCGCT |
| | | AATGCGGGAAAGCTCTTATTCGGGTGAGATGGGCTGGGGCACCAT |
| | | CTGGGGACCCTGACGTGAAGTTTGTCACTGACTGGAGAACTCGGT |
| | | TTGTCGTCTGTTGCGGGGGCGGCAGTTATGGCGGTGCCGTTGGGC |
| | | AGTGCACCCGTACCTTTGGGAGCGCGCGCCCTCGTCGTGTCGTGA |
| | | CGTCACCCGTTCTGTTGGCTTATAATGCAGGGTGGGGCCACCTGC |
| | | CGGTAGGTGTGCGGTAGGCTTTTCTCCGTCGCAGGACGCAGGGTT |
| | | CGGGCCTAGGGTAGGCTCTCCTGAATCGACAGGCGCCGGACCTCT |
| | | GGTGAGGGGAGGGATAAGTGAGGCGTCAGTTTCTTTGGTCGGTTT |
| | | TATGTACCTATCTTCTTAAGTAGCTGAAGCTCCGGTTTTGAACTA |
| | | TGCGCTCGGGGTTGGCGAGTGTGTTTTGTGAAGTTTTTTAGGCAC |
| | | CTTTTGAAATGTAATCATTTGGGTCAATATGTAATTTTCAGTGTT |
| | | AGACTAGTAAATTGTCCGCTAAATTCTGGCCGTTTTTGGCTTTTT |
| | | TGTTAGACGAAGCTAAGGCGCGCCTGAGAACTTCAGGGTGAGTTT |
| | | GGGGACCCTTGATTGTTCTTTCTTTTTCGCTATTGTAAAATTCAT |
| | | GTTATATGGAGGGGGCAAAGTTTTCAGGGTGTTGTTTAGAATGGG |
| | | AAGATGTCCCTTGTATCACCATGCATGGACCCTCATGATAATTTT |
| | | GTTTCTTTCACTTTCTACTCTGTTGACAACCATTGTCTCCTCTTA |
| | | TTTTCTTTTCATTTTCTGTAACTTTTTCGTTAAACTTTAGCTTGC |
| | | ATTTGTAACGAATCTAGACAATCAGGGTATATTATATTGTACTTC |
| | | AGCACAGTTTTAGAGAACAATTGTTATAATTAAATGATAAGGTAG |
| | | AATATTTCTGCATATAAATTCTGGCTGGCGTGGAAATATTCTTAT |
| | | TGGTAGAAACAACTACATCCTGGTCATCATCCTGCCTTTCTCTTT |
| | | ATGGTTACAATGATATACACTGTTTGAGATGAGGATAAAATACTC |
| | | TGAGTCCAAACCGGGCCCCTCTGCTAAC |
| 28 | Intron in | GAGTAGCGGGCTGCTGGGCTGGCCGGGGCTTTCGTGGCCGCCGGG |
| | Ubc-Intron | CCGCTCGGTGGGACGGAAGCGTGTGGAGAGACCGCCAAGGGCTGT |
| | | AGTCTGGGTCCGCGAGCAAGGTTGCCCTGAACTGGGGGTTGGGGG |
| | | GAGCGCAGCAAAATGGCGGCTGTTCCCGAGTCTTGAATAGAACCT |
| | | TCGCTAATGCGGGAAAGCTCTTATTCGGGTGAGATGGGCTGGGGC |
| | | ACCATCTGGGGACCCTGACGTGAAGTTTGTCACTGACTGGAGAAC |
| | | TCGGTTTGTCGTCTGTTGCGGGGGCGGCAGTTATGGCGGTGCCGT |
| | | TGGGCAGTGCACCCGTACCTTTGGGAGCGCGCGCCCTCGTCGTGT |
| | | CGTGACGTCACCCGTTCTGTTGGCTTATAATGCAGGGTGGGGCCA |
| | | CCTGCCGGTAGGTGTGCGGTAGGCTTTTCTCCGTCGCAGGACGCA |
| | | GGGTTCGGGCCTAGGGTAGGCTCTCCTGAATCGACAGGCGCCGGA |
| | | CCTCTGGTGAGGGGAGGGATAAGTGAGGCGTCAGTTTCTTTGGTC |
| | | GGTTTTATGTACCTATCTTCTTAAGTAGCTGAAGCTCCGGTTTTG |
| | | AACTATGCGCTCGGGGTTGGCGAGTGTGTTTTGTGAAGTTTTTTA |
| | | GGCACCTTTTGAAATGTAATCATTTGGGTCAATATGTAATTTTCA |
| | | GTGTTAGACTAGTAAATTGTCCGCTAAATTCTGGCCGTTTTTGGC |
| | | TTTTTTGTTAGACGAAGCTAAGGCGCGCCTGAGAACTTCAGGGTG |
| | | AGTTTGGGGACCCTTGATTGTTCTTTCTTTTTCGCTATTGTAAAA |
| | | TTCATGTTATATGGAGGGGGCAAAGTTTTCAGGGTGTTGTTTAGA |
| | | ATGGGAAGATGTCCCTTGTATCACCATGCATGGACCCTCATGATA |
| | | ATTTTGTTTCTTTCACTTTCTACTCTGTTGACAACCATTGTCTCC |
| | | TCTTATTTTCTTTTCATTTTCTGTAACTTTTTCGTTAAACTTTAG |
| | | CTTGCATTTGTAACGAATCTAGACAATCAGGGTATATTATATTGT |
| | | ACTTCAGCACAGTTTTAGAGAACAATTGTTATAATTAAATGATAA |
| | | GGTAGAATATTTCTGCATATAAATTCTGGCTGGCGTGGAAATATT |
| | | CTTATTGGTAGAAACAACTACATCCTGGTCATCATCCTGCCTTTC |
| | | TCTTTATGGTTACAATGATATACACTGTTTGAGATGAGGATAAAA |
| | | TACTCTGAGTCCAAACCGGGCCCCTCTGCTAAC |
| 29 | CMV | acgcgtctagttattaatagtaatcaattacggggtcattagttc |
| | promoter | atagcccatatatggagttccgcgttacataacttacggtaaatg |
| | | gcccgcctggctgaccgcccaacgacccccgcccattgacgtcaa |
| | | taatgacgtatgttcccatagtaacgccaatagggactttccatt |
| | | gacgtcaatgggtggagtatttacggtaaactgcccacttggcag |
| | | tacatcaagtgtatcatatgccaagtacgccccctattgacgtca |
| | | atgacggtaaatggcccgcctggcattatgcccagtacatgacct |
| | | tatgggactttcctacttggcagtacatctacgtattagtcatcg |
| | | ctattaccatggtgatgcggttttggcagtacatcaatgggcgtg |
| | | gatagcggtttgactcacggggatttccaagtctccaccccattg |
| | | acgtcaatgggagtttgttttggcaccaaaatcaacgggactttc |
| | | caaaatgtcgtaacaactccgccccattgacgcaaatgggcggta |
| | | ggcgtgtacggtgggaggtctatataagcagagctcgtttagtga |
| | | accgtcagatcgcctggagacgccatccacgctgttttgacctcc |
| | | atagaagacaccgggaccgatccagcctcc |
| 30 | CMV- | acgcgtctagttattaatagtaatcaattacggggtcattagttc |
| | Intron (CMV | atagcccatatatggagttccgcgttacataacttacggtaaatg |
| | promoter | gcccgcctggctgaccgcccaacgacccccgcccattgacgtcaa |
| | intron) | taatgacgtatgttcccatagtaacgccaatagggactttccatt |
| | | gacgtcaatgggtggagtatttacggtaaactgcccacttggcag |
| | | tacatcaagtgtatcatatgccaagtacgccccctattgacgtca |
| | | atgacggtaaatggcccgcctggcattatgcccagtacatgacct |
| | | tatgggactttcctacttggcagtacatctacgtattagtcatcg |
| | | ctattaccatggtgatgcggttttggcagtacatcaatgggcgtg |
| | | gatagcggtttgactcacggggatttccaagtctccaccccattg |
| | | acgtcaatgggagtttgttttggcaccaaaatcaacgggactttc |
| | | caaaatgtcgtaacaactccgccccattgacgcaaatgggcggta |
| | | ggcgtgtacggtgggaggtctatataagcagagctcgtttagtga |
| | | accgtcagatcgcctggagacgccatccacgctgttttgacctcc |
| | | atagaagacaccgggaccgatccagcctccgcggattcgaatccc |
| | | ggccgggaacggtgcattggaacgcggattccccgtgccaagagt |
| | | gacgtaagtaccgcctatagagtctataggcccacaaaaaatgct |
| | | ttcttcttttaatatacttttttgtttatcttatttctaatactt |
| | | tccctaatctctttctttcagggcaataatgatacaatgtatcat |
| | | gcctctttgcaccattctaaagaataacagtgataatttctgggt |
| | | taaggcaatagcaatatttctgcatataaatatttctgcatataa |
| | | attgtaactgatgtaagaggtttcatattgctaatagcagctaca |
| | | atccagctaccattctgcttttattttatggttgggataaggctg |
| | | gattattctgagtccaagctaggcccttttgctaatcatgttcat |
| | | acctcttatcttcctcccacagctcctgggcaacgtgctggtctg |
| | | tgtgctggcccatcactttggcaaagaattgggat |
| 31 | Intron in | gcggattcgaatcccggccgggaacggtgcattggaacgcggatt |
| | CMV-Intron | ccccgtgccaagagtgacgtaagtaccgcctatagagtctatagg |
| | | cccacaaaaaatgctttcttcttttaatatacttttttgtttatc |
| | | ttatttctaatactttccctaatctctttctttcagggcaataat |
| | | gatacaatgtatcatgcctctttgcaccattctaaagaataacag |
| | | tgataatttctgggttaaggcaatagcaatatttctgcatataaa |
| | | tatttctgcatataaattgtaactgatgtaagaggtttcatattg |
| | | ctaatagcagctacaatccagctaccattctgcttttattttatg |
| | | gttgggataaggctggattattctgagtccaagctaggccctttt |
| | | gctaatcatgttcatacctcttatcttcctcccacagctcctggg |
| | | caacgtgctggtctgtgtgctggcccatcactttggcaaagaatt |
| | | gggat |
| 32 | CAG | CGTTACATAACTTACGGTAAATGGCCCGCCTGGCTGACCGCCCAA |
| | promoter | CGACCCCCGCCCATTGACGTCAATAATGACGTATGTTCCCATAGT |
| | | AACGCCAATAGGGACTTTCCATTGACGTCAATGGGTGGACTATTT |
| | | ACGGTAAACTGCCCACTTGGCAGTACATCAAGTGTATCATATGCC |
| | | AAGTACGCCCCCTATTGACGTCAATGACGGTAAATGGCCCGCCTG |
| | | GCATTATGCCCAGTACATGACCTTATGGGACTTTCCTACTTGGCA |
| | | GTACATCTACGTATTAGTCATCGCTATTACCATGGGTCGAGGTGA |
| | | GCCCCACGTTCTGCTTCACTCTCCCCATCTCCCCCCCCTCCCCAC |
| | | CCCCAATTTTGTATTTATTTATTTTTTAATTATTTTGTGCAGCGA |
| | | TGGGGGCGGGGGGGGGGGGGGCGCGCGCCAGGCGGGGCGGGGCGG |
| | | GGCGAGGGGCGGGGCGGGGCGAGGCGGAGAGGTGCGGCGGCAGCC |
| | | AATCAGAGCGGCGCGCTCCGAAAGTTTCCTTTTATGGCGAGGCGG |
| | | CGGCGGCGGCGGCCCTATAAAAAGCGAAGCGCGCGGCGGGCG |
| 33 | GOI-C03- | actaggggttcctgcggccgcacgcgtggaattcgctagtta |
| | Forward | |
| | primer | |
| 34 | GOI-C03- | gccacccgtagatctacggtatcatatcgataag |
| | Reverse | |
| | primer | |
| 35 | GOI-C24- | actaggggttcctgcggccgcggcctccgcgccgggttttg |
| | Forward | |
| | primer | |
| 36 | GOI-C24- | gccacccgtagatctctcgagttaggcgcacttgctcacgc |
| | Reverse | |
| | primer | |
| 37 | GOI-C26- | actaggggttcctgcggccgcacgcgtctagttattaatag |
| | Forward | |
| | primer | |
| 38 | GOI-C26- | ccacccgtagatctctcgagcagcgctgctcgaggcaagc |
| | Reverse | |
| | primer | |
| 39 | GOI-C59- | tcactaggggttcctatcgatatcaagcttcgttac |
| | Forward | |
| | primer | |
| 40 | GOI-C59- | agggatgccacccgtggatcctcaggcgcacttg |
| | Reverse | |
| | primer | |
| 41 | GOI-C45- | ttcgaacatcgattgaattctgagccaccatgtccaggaaaatc |
| | Forward | |
| | primer | |
| 42 | GOI-C45- | aggtcgactctagaggatcctcaggcgcacttgctcacac |
| | Reverse | |
| | primer | |
| 43 | GOI-C46- | ttcgaacatcgattgaattctgagccaccatgtacagaatg |
| | Forward | |
| | primer | |
| 44 | GOI-C46- | aggtcgactctagaggatcctcaggcacacttactcacac |
| | Reverse | |
| | primer | |
| 45 | GOI-E11-V | gactctagaggatcgaattcgccaccatgtcacgcaagatag |
| | 1-Forward | |
| | primer | |
| 46 | GOI-E11-V | gggatgccacccgtggatcctcaggcacacttgctgacg |
| | 1-Reverse | |
| | primer | |
| 47 | GOI-C21- | caagttaacaacaacaattgcattcattttatgtttcag |
| | Forward | |
| | primer | |
| 48 | GOI-C21-R | acggccagtgaattgtcgacgcatgcgggcccggatctg |
| | everse | |
| | primer | |
| 49 | Forward | gacgtcagacgcggaagcttcgatc |
| | primer | |
| 50 | Reverse | gctgtttaaacgcccgggctgtag |
| | primer | |
| 51 | AAV2 VP1 | MAADGYLPDWLEDTLSEGIRQWWKLKPGPPPPKPAERHKDDSRGL |
| | Amino acid | VLPGYKYLGPFNGLDKGEPVNEADAAALEHDKAYDRQLDSGDNPY |
| | sequence | LKYNHADAEFQERLKEDTSFGGNLGRAVFQAKKRVLEPLGLVEEP |
| | | VKTAPGKKRPVEHSPVEPDSSSGTGKAGQQPARKRLNFGQTGDAD |
| | | SVPDPQPLGQPPAAPSGLGTNTMATGSGAPMADNNEGADGVGNSS |
| | | GNWHCDSTWMGDRVITTSTRTWALPTYNNHLYKQISSQSGASNDN |
| | | HYFGYSTPWGYFDFNRFHCHFSPRDWQRLINNNWGFRPKRLNFKL |
| | | FNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRT |
| | | GNNFTFSYTFEDVPFHSSYAHSQSLDRLMNPLIDQYLYYLSRTNT |
| | | PSGTTTQSRLQFSQAGASDIRDQSRNWLPGPCYRQQRVSKTSADN |
| | | NNSEYSWTGATKYHLNGRDSLVNPGPAMASHKDDEEKFFPQSGVL |
| | | IFGKQGSEKTNVDIEKVMITDEEEIRTTNPVATEQYGSVSTNLQR |
| | | GNRQAATADVNTQGVLPGMVWQDRDVYLQGPIWAKIPHTDGHFHP |
| | | SPLMGGFGLKHPPPQILIKNTPVPANPSTTFSAAKFASFITQYST |
| | | GQVSVEIEWELQKENSKRWNPEIQYTSNYNKSVNVDFTVDTNGVY |
| | | SEPRPIGTRYLTRNL |

1. An AAV capsid protein variant comprising an engineered capsid protein VP1 comprising the following amino acid mutation sites relative to a parental AAV capsid protein VP1: Q464V, A467P, D469N, 1470M, R471A, D472V, and S474G, wherein the positions are determined with reference to the amino acid sequence position of SEQ ID NO: 51;
optionally, the VP1 further comprises Y500F and/or S501A.

2. The AAV capsid protein variant of claim 1, wherein the parental AAV capsid protein VP1 is from AAV serotype 2 (AAV2) or AAV2 variant version 7m8 (AAV2.7m8), preferably AAV2.7m8.

3. The AAV capsid protein variant of claim 1, wherein the AAV capsid protein variant
(i) comprises the amino acid sequence as set forth in SEQ ID NO:1.
(ii) comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:1 and comprises the following substitutions: Q464V, A467P, D469N, 1470M, R471A, D472V, and S474G;
(iii) is encoded by the nucleic acid sequence as set forth in SEQ ID NO: 2; or
(iv) is encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence set forth in SEQ ID NO:2 and comprises the following substitutions: Q464V, A467P, D469N, 1470M, R471A, D472V, and S474G.

4. An isolated nucleic acid comprising a nucleotide sequence encoding the AAV capsid protein variant according to any one claims 1-3.

5. The isolated nucleic acid according to claim 4, wherein the nucleotide sequence encodes a capsid protein, which
(i) comprises or consists of the nucleic acid sequence as set forth in SEQ ID NO:2; or
(iv) comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:2 and the capsid protein variant encoded by said nucleic acid sequence comprises the following substitutions: Q464V, A467P, D469N, 1470M, R471A, D472V, and S474G.

6. A recombinant AAV viral particle (rAAV) comprising
(i) an AAV capsid protein variant of any one of claims 1-5;
(ii) an interested nucleic acid packaged within the AAV capsid, the interested nucleic acid comprising a nucleic acid molecule encoding RS1.

7. The recombinant AAV viral particle of claim 6, wherein the RS1 is human RS1.

8. The recombinant AAV viral particle of claim 7, wherein the human RS1 protein
(i) comprises or consists of the amino acid sequence as set forth in SEQ ID NO: 15; or
(ii) comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:15.

9. The recombinant AAV viral particle of any one of claims 6-8, wherein the RS1 is encoded by the following coding sequence which
(i) comprises or consists of the nucleotide sequence as set forth in any one of SEQ ID NOs: 16-18 or a complementary sequence thereof; or
(ii) comprises a nucleotide sequence having at least 85%, 86%, 87%, 88%, 89%, 90%, 91%, 92%, 93%, 94%, 95%, 96%, 97%, 98%, or 99% identity to the nucleotide sequence as set forth in any one of SEQ ID NOs: 16-18 or a complementary sequence thereof.

10. The recombinant AAV viral particle according to any of claims 6-9, wherein the interested nucleic acid is comprised in an expression cassette and thus packaged within the AAV capsid.

11. The recombinant AAV viral particle according to claim 10, wherein the expression cassette is single-stranded DNA, double-stranded DNA or single-stranded RNA or double-stranded RNA.

12. The recombinant AAV viral particle of claim 10 or 11, wherein the expression cassette comprises at least one ITR (e.g., L-ITR or R-ITR) sequence flanking the interested nucleic acid.

13. The recombinant AAV viral particle of any one claims 10-12, wherein the expression cassette comprises one or more regulatory sequences selected from one or more or all of a promoter, an inverted repeat sequence, an intron, an enhancer, a post-transcriptional regulatory sequence, a polyadenylation region, a selectable marker, or a reporter gene.

14. A method of producing a recombinant AAV viral particle, comprising culturing a packaging cell under conditions sufficient for production of a recombinant AAV viral particle, wherein the packaging cell comprises a plasmid comprising a nucleic acid encoding the capsid protein variant of any one of claims 1-3 or the nucleic acid of claim 4 or 5.

15. The method according to claim 14, wherein the packaging cell further comprises a helper plasmid and/or a transfer plasmid comprising the interested nucleic acid.

16. The method according to claim 14 or 15, further comprising isolating from the culture supernatant a self-complementary recombinant AAV viral particle.

17. The method according to any one of claims 14-16, further comprising lysing the packaging cell, and isolating a recombinant AAV viral particle from the cell lysate.

18. The method according to any one of claims 14-17, further comprising one or more of the following steps:
a. removing the cell debris,
b. treating the supernatant containing the recombinant AAV viral particle with a benzonase nuclease,
c. concentrating the recombinant AAV viral particle,
d. purifying the recombinant AAV viral particle.

19. A recombinant AAV viral particle prepared according to the method of any one of claims 14-18.

20. A packaging cell for producing a recombinant AAV viral particle, the packaging cell comprising a plasmid comprising a nucleic acid encoding the capsid protein variant according to any one of claims 1-3 or the nucleic acid of claim 4 or 5.

21. A formulation or composition or medicament comprising the recombinant AAV viral particle of any one claim 6-13 or 19, and optionally pharmaceutically acceptable auxiliary material, such as pharmaceutically acceptable carriers, excipients, including buffers, as known in the art.

22. A combination product comprising the recombinant AAV viral particle of any one of claim 6-13 or 19, and one or more additional therapeutic agents, e.g., an immunomodulator, such as, an immunosuppressant.

23. A method of treating an ocular disease in an individual comprising administering to the individual the recombinant AAV viral particle of any one of claim 6-13 or 19 or the formulation or composition or medicament of claim 21 or the combination product of claim 22.

24. The method of claim 23, wherein the ocular disease is selected from the group consisting of diseases caused by retinopathy or chorioretinopathy, age-related macular degeneration, diabetic retinopathy, and other non-hereditary ophthalmic diseases such as physically or chemically induced retinal damage, e.g., X-linked retinoschisis or retinitis pigmentosa.

25. The method of claim 23 or 24, wherein the administering is via intraocular administration, e.g., by intraretinal administration or intravitreal administration, e.g., subretinal administration or intravitreal administration (IVT).

26. The method of claim 25, wherein the administering is injection.

27. The method of any one of claims 23-26, wherein the recombinant AAV viral particle is administered at a dose of greater than or equal to about 10³vg/eye/dose, 10⁴vg/eye/dose, 10⁵vg/eye/dose, 10⁶vg/eye/dose, 10⁷vg/eye/dose, 10⁸vg/eye/dose, 10⁹vg/eye/dose, 10¹⁰ vg/eye/dose, 10¹¹vg/eye/dose, or 10¹²vg/eye/dose, preferably no more than 10¹²vg/eye/dose, for example, greater than or equal to 2×10⁸vg/eye/dose, 3×10⁸vg/eye/dose, 4×10⁸vg/eye/dose or 5×10⁸vg/eye/dose, for example, between about 10⁷vg/eye/dose and about 5×10⁸vg/eye/dose, between 1×10⁷vg/eye/dose and about 5×10⁸vg/eye/dose, for example, about 2×10⁸vg/eye/dose, 3×10⁸vg/eye/dose, 4×10⁸vg/eye/dose or 5×10⁸vg/eye/dose.
